(19) United States
(12) Patent Application Publication
(10) Pub. No.: US 2015/0272922 A1
(43) Pub. Date: Oct. 1, 2015

(54) Compounds for Reducing Glucocorticoids, and Methods of Treatment Thereof

(71) Applicants: BIONICHE LIFE SCIENCES INC., Belleville (CA); UNIVERSITY OF OTTAWA, Ottawa (CA)

(72) Inventors: Tony Durst, Ottawa (CA); Zulfiquar Merali, Ottawa (CA); Christian Cayer, Cantley (CA); John Thor Arnason, Ottawa (CA); John Douglas Baker, Stirling (CA)

(21) Appl. No.: 14/441,620

(22) PCT Filed: Nov. 8, 2013
(86) PCT No.: PCT/CA2013/000949

§ 371 Date: May 8, 2015

Related U.S. Application Data
(60) Provisional application No. 61/724,409, filed on Nov. 9, 2012.

(51) Int. Cl. A61K 31/235; A61K 31/19; A61K 31/045; A61K 31/215

(52) U.S. Cl. CPC A61K 31/235; A61K 31/215; A61K 31/19; A61K 31/045

(57) ABSTRACT

A method for reducing glucocorticoids in an animal in need thereof comprising the use of compounds of the formula (I), wherein the definitions for R′, R₁-R₁₁ and n are as disclosed in the description. The compounds of formula (I) are for the treatment or prevention of a glucocorticoid-related disorder for maintaining bone density, maintaining and improving the immune system, treating Cushing's syndrome, treating obesity, improving reproduction efficiency, treating metabolic disorder, treating hypertension, treating hyperglycemia, treating insulin resistance, treating type 2 diabetes, and/or aiding in cancer and immune therapies

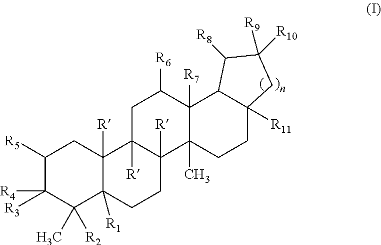

### RELATED APPLICATIONS

This application claims priority to U.S. Provisional Patent Application No. 61/724,409 filed on Nov. 9, 2012, the content of which is hereby incorporated by reference in its entirety.

### FIELD

This disclosure relates to methods for reducing glucocorticoids in animals in need thereof.

### BACKGROUND

In the vertebrates, the perception of stress initiates a response cascade involving the hypothalamus-pituitary-adrenal (HPA, in mammals) or interrenal (HPI, in fish) axis. The HPA axis is a major part of the neuroendocrine system that controls reactions to stress and regulates many body processes. When stress is perceived, responses occur in these stress axes, which stimulates the release of corticotrophin-releasing-factor (CRF) from the hypothalamus. This in turn stimulates the release of adrenocorticotropic hormone (ACTH) from the pituitary gland. ACTH circulates in the blood and stimulates the release of cortisol (corticosterone in rodents) from the adrenal cells (or head kidney cells in fish) into the blood stream. Cortisol is the primary signaling molecule in the HPA axis. Cortisol levels are elevated in animals in response to stress, which leads to physiological changes that can help the animal respond to stress, for example by an enhanced flight or fight response. Cortisol mediates a host of physiological response such as increased catabolism, aggression and down regulation of other functions such as immune responses and reproduction function in mammals. In stress trials, circulating plasma cortisol (corticosterone) is considered a common biomarker to confirm that a stimulus exerts significant stress and to distinguish non-stressed from stressed animals (Gamperl et al., 1994).

These responses are thought to be a short term biological adaptation to stress in animals, however, chronic stress, which can lead to chronically elevated cortisol, hypercortisolism, is maladaptive, and is implicated in disease in both animals and humans. In livestock, elevated stress slows growth and reduces production (Rostagno, 2009). Stress is a concern in aquaculture. The stress response in fish leads to mobilization of energy-rich substrates by depletion of hepatic glycogen stores, elevation of plasma glucose, changes in circulating free fatty acid levels and general inhibition of protein synthesis. These responses have a catabolic effect on fish. Therefore in aquaculture, elevated cortisol adversely affects growth rate, immunity and reproduction (Schreck et al., 2001). In animal production in agriculture, juvenile pigs experience stress when weaned and moved into common pens, elevating cortisol. As a result of their elevated cortisol, they have lower weight gain and their high aggression and lower immune function leads to fights, injuries and more infections. In humans, stress and stress-related illnesses are widespread among people and increasingly a link has been identified between elevated plasma cortisol levels and heart disease, obesity, metabolic syndrome, hyperglycermia (Brown D F et al. 2003). Hypercortisolemia is associated with severity of bone loss and depression in hypothalamic Amenorrhea and Anorexia Nervosa (Lawson et al. 2009) and depression (Anagnostis et al., 2009; Carroll et al., 2007; Gathercole and Stewart, 2010; Smith et al., 2005 Parker et al., 2003). In humans, diverse stressful stimuli, including low socioeconomic status, race (Hajat et al., 2010), chronic work stress (Chandola et al., 2006), anxiety and depression (Carroll et al., 2007) stimulate neuroendocrine responses. Pain caused by muscular problems (tension headaches, back and jaw pain, repetitive stress syndrome), gastrointestinal disorders (heart burn, diarrhea, stomach pain) (Anagnostis 2009), mental disorders (eating disorders, anxiety, depression, schizophrenia, insomnia, substance abuse) (Parker 2003) can also stimulate neuroendocrine responses. Stress related behaviour (for example cowering, licking, circling, digging and chewing in dogs) when facing a stressful stimuli such as, transportation, veterinarian visits, vaccination and drug treatments can also result in a neuroendocrine response (Herron 2008). Animals raised in large scale industrial farms are housed in an environmentally stressful context and express high levels of glucocorticoids, resulting in reduced reproductive efficiency and limited yield in overall productivity.

Notwithstanding the link between hypercortisolism and a variety of human illnesses, pharmacological treatments for hypercortisolism are still under investigation. The hypercortisolism present in Cushing's syndrome is typically caused by a tumor (adrenal or pituitary) and treated with surgery (Sharma and Nieman, 2011). The interest in the role of hypercortisolism in metabolic syndrome stems from the phenotypic similarities between patients with Cushing's and metabolic syndrome. Both include central obesity, impaired glucose tolerance, insulin resistance, type-two diabetes, increased cardiac risk of mortality, osteoporosis and depression (Gathercole and Stewart, 2010). Normalizing cortisol levels usually reverses the symptoms in Cushing's syndrome (Stewart, 2003).

Stress and stress-related illness is widespread, and links between elevated plasma cortisol and heart disease (Smith et al., 2005), obesity (Travison et al., 2007) and depression (Carroll et al., 2007; Parker et al., 2003) have been reported. Aberrations in HPA axis function, including hypercortisolism, are strongly associated with depression (Gallagher et al., 2008). Drugs that inhibit cortisol synthesis, including ketoconazole, aminoglutethimide and metyrapone, have been examined for their therapeutic potential in treating depression (Kling et al., 2009; Starkman et al., 2001) and have shown some promise in bipolar patients with depressive symptoms (ketoconazole) (Brown et al., 2001), and patients with major depressive disorder (MDD). In a blind, placebo-controlled study with patients suffering from MDD, co-delivery of metyrapone (an inhibitor of cortisol synthesis by blocking the mitochrondrial steroidogenic enzyme steroid 11-13 hydroxylase) and a standard serotonergic antidepressant (nefazodone or fluvoxamine), significantly reduced depression (50% reduction in HAM-D scores at day 35 of treatment) (Jahn et al., 2004). However, despite the potential of cortisol-lowering drugs to treat depression, they are also associated with serious side-effects (Thomson and Craighead, 2008), including ketoconazole's potential for liver toxicity (Kim et al., 2003b) and strong inhibition of cytochrome P450 3A (Cook et al., 2004), with the consequence that in some clinical trials up to 20% of patients drop out due to the side-effects (Wolkowitz et al., 1999).

In the context of metabolic syndrome, inhibition of the enzyme 11β hydroxysteroid dehydrogenase 1 (11-β HSD1) to treat hypercortisolism has been examined (Gathercole and Stewart, 2010). At the pre-receptor level, 11-β HSD1 converts metabolically inactive cortisone to active cortisol (11-dehydrocorticosterone to corticosterone in rodents). In rodent models of metabolic syndrome, inhibition of 11-β HSD1 improves metabolic profile (Gathercole and Stewart, 2010). A recent Phase I clinical trial with a selective 11-β HSD1 inhibitor shows good tolerability and no activation of the HPA axis in healthy patients (Courtney et al., 2008).

Anxiety is a serious disorder that affects many people. Anxiety disorders can be classified into the following sub-categories: generalized anxiety disorder, panic disorders, phobias, obsessive-compulsive disorders, posttraumatic stress disorder, acute stress disorders and anxiety disorders due to medical conditions, substance abuse and not otherwise specified anxiety (American Psychiatric Association. (1994). Diagnostic and statistical manual of mental disorders, 4^{th} Ed. (DSM-IV). Washington, D.C.). Anxiety disorders are characterized by three basic components; subjective psychological reports, behavioral responses and physiological responses. A person usually reports subjective feelings of tension, apprehension, dread and expectations of an inability to cope (Alloy, L. B., Jacobson, N. S, & Acocella, J., (1999). Abnormal Psychology: Current Perspectives (pp. 150-172.). McGraw-Hill, Boston Mass.). These feelings can lead the person to behavioral responses as coping mechanisms, such as avoidance of the feared situation, impaired speech and motor functioning, and impaired performance on complex cognitive tasks. Physiological changes are often manifested as well; these include muscle tension, increased heart rate and blood pressure, dry mouth, nausea and dizziness (Gray and McNaughton, 2000; Steimer 2011; Edwards 1991).

Post Traumatic Stress Disorder (PTSD) is an anxiety disorder that results from exposure to a traumatic event. The disabling psychological symptoms associated with PTSD can occur long after the exposure to the traumatic event(s). The DSM-V encompasses several symptoms related to the disorder, including numbing, avoidance, increased arousal and it is often associated re-experiencing of the past trauma. PTSD symptoms elicit severe distress and/or impair normal functioning. PTSD has become a global health issue, with prevalence rates ranging from 1.3% to 37.4%. As PTSD is often accompanied with other health conditions, it is difficult to appropriately pinpoint a specific treatment regimen for each individual of them. Serotonin reuptake inhibitors (SSRIs) or serotonin/norepinephrine reuptake inhibitors (SNRIs) and benzodiazepines, are typically used to treat PTSD. However, the outcomes are variable, as majority of affected individuals do not experience full remission. Furthermore, there are a number of side effects (e.g. sedation, decreased libido, weight gain) with these drugs. The usage of anxiolytic plants for treating the symptoms associated with PTSD has been suggested by Maddox et al. (2013), Passie et al. (2012) and Sarris et al. (2013).

Conditioned fear is relevant to Post Traumatic Stress Disorder. Pavlovian fear conditioning (FC) is an important paradigm for studying memory processes and related brain circuits that are relevant to PTSD (Cain et al., 2012; Parson et al. 2013). Models of FC in rodents include conditioned emotional response (CER) and fear potentiated startle paradigm (FPS). FC occurs when a neutral stimulus (e.g. light or tone) is conditioned (CS) or temporally paired with naturally aversive unconditioned stimuli (US; foot shock). Typically, after a number of repeated pairings, the CS itself acquires the capacity to elicit responses akin to that of US, that is a fear response. Thus, FC is an extremely powerful form of associative learning; if the CS is sufficiently salient and the US sufficiently aversive, even a single brief episode can lead to strong fear memories that can last a lifetime. FC studies have greatly enhanced our understanding of the neurobiology of memory and emotional responding. Although FC does not necessarily produce PTSD, psychological and neural processes mediating FC likely engage brain circuits and processes that contribute to PTSD. FC has several important features and phases that may make distinct contributions to PTSD or treatment (Cain et al., 2012; Parson et al. 2013).

Acquisition refers to the process by which the organism learns that the conditioned stimulus predicts the unconditioned stimulus. Treatments that block the acquisition of fear conditioning are applied before CS-US pairings and prevent the development of short-term memory (STM) memory, tested within a few hours, and consequently the formation of long-term memory (LTM), tested many hours or days later.

The consolidation of fear conditioning refers to the transformation of the short-term memory from a labile state immediately after acquisition to a more permanent state with the passage of time. Treatments that disrupt the consolidation of memory are usually applied a few minutes to a few hours after CS-US pairings, leaving STM intact but resulting in LTM disruption.

Retrieval (also refers to as expression) refers to the conditioned responding that occurs after CS-US relationship has been established. Retrieval is assessed by presenting the CS alone and then measuring the conditioned response. Treatments that disrupt retrieval are typically administered shortly before the conditioned response is assessed. Drugs that disrupt retrieval may blunt PTSD symptoms.

PTSD research has been focused on two other phases on CF, namely reconsolidation and extinction. Reconsolidation occurs after retrieval. During a brief window of time after the retrieval of the fearful memories, the specific memory and its association with an emotional response, return to an unstable state, during which time the “repackaging” of the memory is vulnerable to pharmacological intervention. Reconsolidation of memory is considered to be disrupted when a drug is applied shortly after retrieval and leaves STM intact yet disrupts LTM.

Stress is of particular concern in meat farming and aquaculture. The primary stress response leads to mobilization of energy-rich substrates by depletion of hepatic glycogen stores, elevation of plasma glucose, changes in circulating free fatty acid levels and general inhibition of protein synthesis. These responses mean that stress has an overall catabolic effect on mammals and fish, and in an aquaculture setting elevated cortisol levels adversely affect growth rate, immunity and reproduction (Schreck et al., 2001), and in mammals, there is high mortality associated with transportation or separation stress.

Although the anxiety and stress can be interconnected in subjects, they are separate independent events. Anxiety and stress have different core physiological mechanisms. Mediation of an anxiety response involves many interconnected systems including the GABAergic system, Corticotropin releasing hormone (CRH) and the serotoninergic systems. Stress involves cortisol biosynthesis and activation of the glucocorticoid receptor, with the cascading physiological and neurochemical effects, with cross-activation of the above-mentioned systems.

### SUMMARY

This application relates to methods of reducing glucocorticoids, such as cortisol, in an animal in need thereof. In one embodiment, the present disclosure includes a method for reducing glucocorticoids in an animal in need thereof, comprising administering to the animal a therapeutically effective amount of a compound of the formula (I):

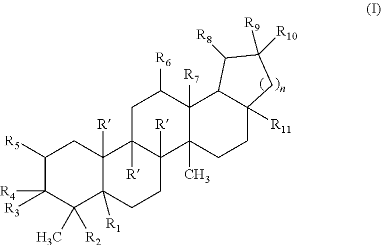

wherein
R′ are each independently or simultaneously H or CH₃;

### R₁ is H or CH₃;

### R₂ is H, CH₃ or CH₂OH;

R₃ is OH, —OCH₃, —OC(O)—(C₁₋₆)alkyl, —OC(O)—(C₆₋₁₀)aryl, —OC(O)—(C₆₋₁₀ heteroaryl, —NC(O)—(C₁₋₆)alkyl, —NC(O)-benzyl;
R₄ is H, —CH₃, (C₆-C₁₀)aryl or (C₆₋₁₀)heteroaryl; or
R₃ and R₄ are joined together to form C═O;
R₅ is H, OH, (C₁₋₆)alkyl or (C₂₋₆)alkenyl;
R₆ and R₇ are independently or simultaneously H or CH₃, or R₆ and R₇ are joined together to form a double bond (C═C);
R₈ is H, (C₁₋₆)alkyl, (C₂₋₆)alkenyl, (C₁₋₆)alkanol moiety, (C₂₋₆)alkenol moiety, —C(O)—(C₁₋₆)alkyl, —C(O)OH, —C(O)O—(C₁-C₆)alkyl, —C(O)—CH₂CH₂—(C₆-C₁₀)aryl, —C(O)—CH₂CH₂—(C₆-C₁₀)heteroaryl, —C(O)—(C₂-C₆)alkenyl, —C(O)—CH₂—CH═CH—(C₆-C₁₀)aryl, —C(O)—CH₂—CH═CH—(C₆-C₁₀)heteroaryl,

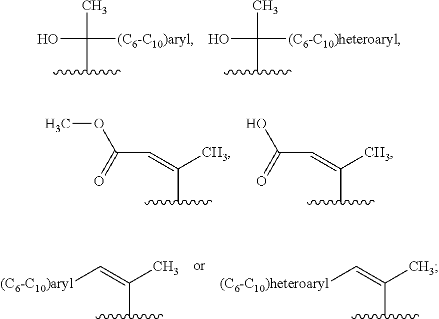

R₉ and R₁₀ are independently or simultaneously H or CH₃;
R₁₁ is CH₃, CH₂OH, CH₂OCOCH₃, —CH(O), —C(O)ORₐ, —C(O)O—(C₁₋₆)alkyl-C(O)O—R_{b}, C(O)—NH—(C₁₋₆)alkyl-C(O)O—R_{b}; or —CH═CH—C(O)O—R_{b};
wherein Rₐ is H, (C₁₋₁₀)alkyl, (C₁₋₆)alkenyl, —CH₂—(C₆-C₁₀)-aryl or —CH₂—(C₆-C₁₀)-heteroaryl,
R_{b} is H or (C₁₋₆)alkyl;
n is the integer 1 or 2; and
the aryl groups are optionally substituted with one to five substituents selected from halo, OH or (C₁₋₃)alkyl,
or a pharmaceutically acceptable salt or solvate thereof, and any stereoisomer (entantiomer, diastereomer) thereof,
with the proviso that the compound of the formula (I) is not

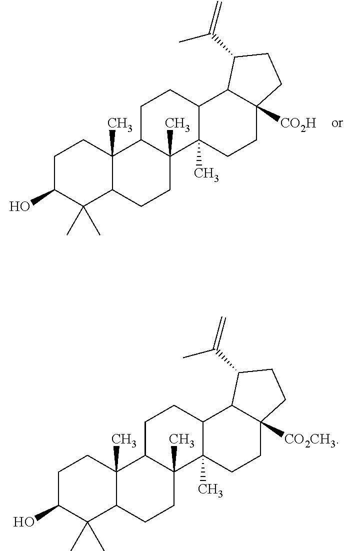

In another embodiment, the present disclosure includes a use of a therapeutically effective amount of a compound of the formula (I) for reducing glucocorticoids in an animal in need thereof,

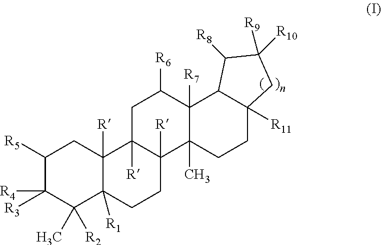

wherein
R′ are each independently or simultaneously H or CH₃;

### R₁ is H or CH₃;

### R₂ is H, CH₃ or CH₂OH;

R₃ is OH, —OCH₃, —OC(O)—(C₁₋₆)alkyl, —OC(O)—(C₆₋₁₀)aryl, —OC(O)—(C₆₋₁₀)heteroaryl,
—NC(O)—(C₁₋₆)alkyl, —NC(O)-benzyl;
R₄ is H, —CH₃, (C₆-C₁₀)aryl or (C₆₋₁₀)heteroaryl; or
R₃ and R₄ are joined together to form C═O;
R₅ is H, OH, (C₁₋₆)alkyl or (C₂₋₆)alkenyl;
R₆ and R₇ are independently or simultaneously H or CH₃, or R₆ and R₇ are joined together to form a double bond (C═C);
R₈ is H, (C₁₋₆)alkyl, (C₂₋₆)alkenyl, (C₁₋₆)alkanol moiety, (C₂₋₆)alkenol moiety, —C(O)—(C₁₋₆)alkyl, —C(O)OH, —C(O)O—(C₁-C₆)alkyl, —C(O)—CH₂CH₂—(C₆-C₁₀)aryl, —C(O)—CH₂CH₂—(C₆-C₁₀)heteroaryl, —C(O)—(C₂-C₆)alkenyl, —C(O)—CH₂—CH═CH—(C₆-C₁₀)aryl, —C(O)—CH₂—CH═CH—(C₆-C₁₀)heteroaryl,

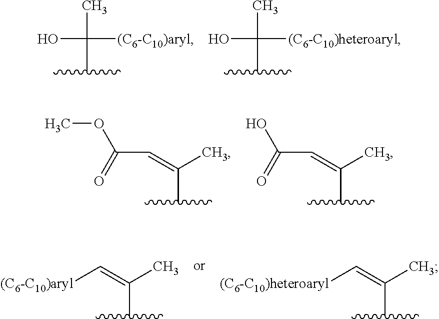

R₉ and R₁₀ are independently or simultaneously H or CH₃;
R₁₁ is CH₃, CH₂OH, CH₂OCOCH₃, —CH(O), —C(O)ORₐ, —C(O)O—(C₁₋₆)alkyl-C(O)O—R_{b}, C(O)—NH—(C₁₋₆)alkyl-C(O)O—R_{b}; or —CH═CH—C(O)O—R_{b};
wherein Rₐ is H, (C₁₋₁₀)alkyl, (C₁₋₆)alkenyl, —CH₂—(C₆-C₁₀)-aryl or —CH₂—(C₆-C₁₀)-heteroaryl,
R_{b} is H or (C₁₋₆)alkyl;
n is the integer 1 or 2; and
the aryl groups are optionally substituted with one to five substituents selected from halo, OH or (C₁₋₃)alkyl,
or a pharmaceutically acceptable salt or solvate thereof, and any stereoisomer (entantiomer, diastereomer) thereof,
with the proviso that the compound of the formula (I) is not

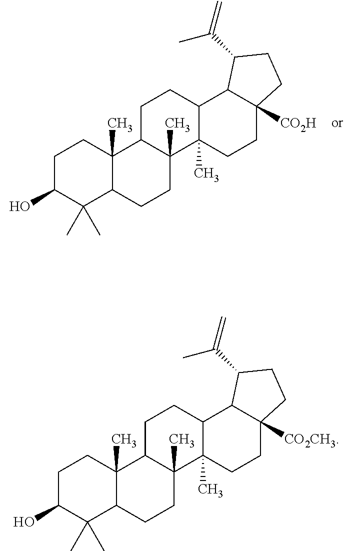

In one embodiment, the compounds of the formula (I), alone or in combination with pharmaceutically acceptable excipients and/or carriers, are administered to subjects in need thereof to reduce the levels of glucocorticoids in the animal.

In one embodiment, the methods and uses for reducing glucocorticoids, such as cortisol or corticosterone, are useful in the treatment of an elevated glucocorticoid-related condition, such as but not limited to, stress, to maintain bone density, to maintain and improve the immune system, to treat Cushing's syndrome, to treat obesity, to improve reproduction efficiency, to reduce aggression and hyperactivity, to treat metabolic disorder, to treat hypertension, to treat hyperglycemia, to treat insulin resistance, to treat type 2 diabetes, and/or to aid in cancer and immune therapies. In another embodiment, the glucocorticoid related condition is stress related or results in stress.

In one embodiment, the animal is human, horse (equine), pig (susidae), sheep, goat, farmed fish (e.g. salmonids, catfish etc.) or crustacean (e.g. shrimp, prawns), bird (e.g. turkeys, fowls, chickens), cattle, endangered or captive species (e.g. zoo animal or aquarium animal) or pet animal (e.g. dog (canine), cat (feline)). In another embodiment, the animal is a human.

Further aspects and advantages of the embodiments described herein will appear from the following description taken together with the accompanying drawing.

### BRIEF DESCRIPTION OF THE DRAWINGS

For a better understanding of the embodiments described herein and to show more clearly how they may be carried into effect, reference will now be made, by way of example only, to the accompanying drawing which shows at least one exemplary embodiment, and in which:

FIG. 1 is a bar graph showing the percentage change of cortisol release in trout head kidney tissue upon administration of compounds of the formula (I);

FIG. 2 is a second bar graph showing the percentage change of cortisol release in trout head kidney tissue upon administration of a compound of the formula (I);

FIG. 3 are graphs showing the percent change in ACTH stimulated cortisol release in trout head kidney tissue after administration of compounds of the disclosure; and

FIG. 4 is a graph showing the relationship between Log P and log cortisol response in trout head kidney tissue after administration of compounds of the disclosure;

FIG. 5 is a graph showing relative corticosterone values obtained from venepuncture blood samples collected from rats after acute mild restraint following a single oral administration of compounds of the disclosure;

FIG. 6 is a graph showing relative corticosterone values obtained from venepuncture blood samples collected from rats after acute mild restraint following 3 consecutive daily oral administration of compound of the disclosure.

FIG. 7a is a graph showing the effects of isolated compounds on total freezing time (%) in rats in the contextual conditioned emotional response test following administration of compounds of the disclosure (7a); FIG. 7b is a graph showing a breakdown of the (%) freezing time in rats in the contextual conditioned emotional response test following administration of compounds of the disclosure; and

FIG. 8a is a graph showing the effects of compounds on the percentage of time spent in open arms in rats in the elevated plus maze following administration of their respective treatments; FIG. 8b shows the effects of compounds on the number of unprotected head dips in rats in the elevated plus maze following administration of their respective treatments.

### DETAILED DESCRIPTION

### (I) Definitions

The term “reducing glucocorticoid” as used herein refers to the reducing of the level of glucocorticoid, such as cortisol or corticosterone, circulating in the animal compared to an untreated control. A control can be an untreated animal or a reference standard.

The term “glucocorticoid” as used herein refers to predominant glucocorticoid affecting the animal, such as cortisol or corticosterone. The predominant glucocorticoid in humans (and most mammals and fish) is cortisol (hydrocortisone) whereas corticosterone is the common glucocorticoid in rodents. All animals produce both hormones. Corticosterone is the precursor of the mineralocorticoid, aldesterone.

The term “animal” as used herein refers to all members of the animal kingdom, including without limitation, humans, horse (equine), pig (susidae), sheep, goat, farmed fish (e.g. salmonids, catfish etc.) or crustacean (e.g. shrimp, prawns), bird (e.g. turkeys, fowls, chickens), cattle, endangered or captive species (e.g. zoo animal or aquarium animal), pet animal (e.g. dog (canine), cat (feline)).

The phrase “therapeutically effective amount” as used herein refers to the amount of the compound of the formula (I), which provides a therapeutic benefit by reducing glucocorticoids. For example, when a compound of the formula (I) of the disclosure is formulated for reducing glucocorticoids, the therapeutically effective amount refers to an amount of the therapeutic agent which lowers the glucocorticoids, and therefore, treats an underlying condition. Different therapeutically effective amounts may be applicable for each disorder, as will be readily known or determined by those of ordinary skill in the art.

The term “C₁₋ₙalkyl” as used herein means straight and/or branched chain, saturated alkyl groups containing from “1” to “n” carbon atoms and includes (depending on the identity of n) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable n is an integer representing the highest number of carbon atoms.

The term “C₂₋ₘalkenyl” as used herein means straight and/or branched chain, unsaturated alkyl groups containing from two to m carbon atoms and one to three double bonds, and includes (depending on the identity of m) vinyl, allyl, 2-methylprop-1-enyl, but-1-enyl, but-2-enyl, but-3-enyl, 2-methylbut-1-enyl, 2-methylpent-1-enyl, 4-methylpent-1-enyl, 4-methylpent-2-enyl, 2-methylpent-2-enyl, 4-methylpenta-1,3-dienyl, hexen-1-yl and the like, where the variable m is an integer representing the largest number of carbon atoms in the alkenyl group.

The term “(C₆-C₁₀)-aryl” as used herein means a monocyclic or polycyclic (such as bicyclic) aromatic ring system containing from 6 to 10 carbon atoms and includes phenyl, naphthyl, 1,2-dihydronaphthyl, 1,2,3,4-tetrahydronaphthyl, indanyl, indenyl and the like.

The term “(C₆-C₁₀)-heteroaryl” as used herein means a monocyclic or polycyclic (such as bicyclic) aromatic ring system containing from 6 to 10 carbon atoms in which one or more (for example, one, two or three) of the atoms in the ring have been replaced by nitrogen, oxygen or sulfur, and includes, indolyl, benzofuranyl, benzothiophenlyl, quinolonyl, isoquinolinyl, pyridyl, furyl, pyrrolyl, thienyl, isothiazolyl, imidazolyl, benzimidazolyl, tetrazolyl, pyrazinyl, pyrimidyl, and the like.

The term “C₁₋₆alkanol” as used herein means straight and/or branched chain, saturated alkyl groups with a hydroxyl (OH) substituent and containing from “1” to “6” carbon atoms and includes a methanol moiety (—CH₂OH), an ethanol moiety (—CH₂CH₂OH), a propanol moiety (—CH₂CH₂CH₂OH), an isopropanol moiety (—CH₂CH(OH)CH₃), etc. Likewise, the term “C₁₋₆dialkanol” refers to the same groups having two hydroxyl substituents.

The term “C₂₋₆alkenol” as used herein means straight and/or branched chain, unsaturated alkyl groups with a hydroxyl (OH) substituent, containing from 2 to 6 carbon atoms and one to three double bonds and includes a propenol moiety (—CH═CHCH₂OH), an s-butenol moiety (CH₂═CHCH(OH)CH₃), an pentenol moiety (CH₂═CHCH₂CH(OH)CH₃), and the like.

The suffix “ene” added on to any of the above groups means that the group is divalent, i.e. inserted between two other groups. When the group is a ring system, the two other groups may be located at any location on the ring system, including at adjacent and non-adjacent nodes.

The term “pharmaceutically acceptable salt” means an acid addition salt or a base addition salt which is suitable for, or compatible with, the treatment of subjects.

An acid addition salt which is suitable for, or compatible with, the treatment of subjects as used herein means any non-toxic organic or inorganic salt of any basic compound. Basic compounds that form an acid addition salt include, for example, compounds comprising an amine group. Illustrative inorganic acids which form suitable salts include hydrochloric, hydrotrifluoroacetic, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art. In an embodiment, the acid addition salt is a hydrochloride or hydrotrifluoroacetic acid salt.

A base addition salt which is suitable for, or compatible with, the treatment of subjects as used herein means any non-toxic organic or inorganic base addition salt of any acidic compound. Acidic compounds that form a base addition salt include, for example, compounds comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

The term “stereoisomer,” as used herein, refers to all possible different isomeric as well as conformational forms which a compound of the formula (I) may possess, including all possible stereochemically and conformationally isomeric forms, all diastereomers, enantiomers and/or conformers of the basic molecular structure.

### (II) Methods of Reducing Glucocorticoids

The present disclosure relates to a method for reducing glucocorticoids in an animal in need thereof. In an embodiment, the glucocorticoid is lowered by 10%, 20%, 30%, 50%, 75%, or more compared to the control glucocorticoid levels.

Reduction of glucocorticoid, such as cortisol/corticosterone, provides a number of benefits to an animal. The methods and uses for reducing glucocorticoid are useful for treating glucocorticoid-related conditions. The term “glucocorticoid-related condition” as used herein refers to any condition that is caused by or affected negatively by an increase in glucocorticoid levels, such as cortisol or corticosterone levels, compared to an unaffected or healthy animal. Accordingly, in an embodiment, the methods and uses for reducing glucocorticoid are used to treat a glucocorticoid-related condition, including without limitation, maintaining bone density, maintaining and improving the immune system, treating Cushing's syndrome, treating obesity or controlling weight gain, improving reproduction efficiency, reducing aggression and hyperactivity, treating metabolic disorder, treating hypertension, treating hyperglycemia, treating insulin resistance, treating type 2 diabetes, and/or aiding in cancer and immune therapies. In one embodiment, the glucocorticoid related disorder is stress related or results in stress.

Accordingly in another aspect, the present disclosure provides a method of treating stress comprising administering one or more compounds of the formula (I) to an animal in need thereof. Also provided herein is use of one or more compounds of the formula (I) for treating stress in an animal in need thereof. Further provided is use of one or more compounds of the formula (I) in the preparation of a medicament for treating stress in an animal in need thereof. Even further provided is one or more compounds of the formula (I) for use in treating stress in an animal in need thereof.

In yet another aspect, the present disclosure provides a method of reducing glucocorticoid, such as cortisol or corticosterone, comprising administering one or more compounds of the formula (I) to an animal in need thereof. Also provided herein is use of one or more compounds of the formula (I) for reducing glucocorticoid, such as cortisol or corticosterone, in an animal in need thereof. Further provided is use of one or more compounds of the formula (I) in the preparation of a medicament for reducing glucocorticoid, such as cortisol or corticosterone, in an animal in need thereof. Even further provided is one or more compounds of the formula (I) for use in reducing glucocorticoid, such as cortisol or corticosterone, in an animal in need thereof.

High cortisol is known to result in weak, brittle and fragile bones and can shift calcium from the bones to the blood. Accordingly, in another embodiment, the present disclosure provides a method of maintaining or improving bone density comprising administering one or more compounds of the formula (I) as defined herein. Also provided herein is a use of one or more compounds of the formula (I) disclosed herein for maintaining or improving bone density in an animal in need thereof. Further provided is use of one or more compounds of the formula (I) in the preparation of a medicament for maintaining or improving bone density in an animal in need thereof. Even further provided is one or more compounds of the formula (I) for use in maintaining or improving bone density in an animal in need thereof.

High cortisol is also implicated in suppressing the immune system. Thus, in an embodiment, reduction of cortisol is useful for maintaining or improving an active immune system. Accordingly, also provided herein is a method of maintaining or improving an active immune system comprising administering one or more compounds of the formula (I) to an animal in need thereof. Also provided herein is use of one or more compounds of the formula (I) for maintaining or improving an active immune system in an animal in need thereof. Further provided is use of one or more compounds of the formula (I) in the preparation of a medicament for maintaining or improving an active immune system in an animal in need thereof. Even further provided is a use of one or more compounds of the formula (I) for use in maintaining or improving an active immune system in an animal in need thereof.

Cushing's syndrome is caused by overactive adrenal glands which stimulates the production of high levels of cortisol under the influence of increased production of ACTH. Thus, in another embodiment, methods and uses of reducing cortisol are useful for treating Cushing's syndrome. Accordingly, also provided herein is a method of treating Cushing's syndrome comprising administering one or more compounds of the formula (I) to an animal in need thereof. Also provided herein is use of one or more compounds of the formula (I) for treating Cushing's syndrome in an animal in need thereof. Further provided is use of one or more compounds of the formula (I) in the preparation of a medicament for treating Cushing's syndrome in an animal in need thereof. Even further provided is one or more compounds of the formula (I) for use in treating Cushing's syndrome in an animal in need thereof.

High cortisol levels are associated with deposition of fat in the stomach area for storage (visceral obesity). High cortisol levels also increase motivation (via brain reward mechanisms) to consume calorie-dense (high sugar/high fat content) foods (Dallman, M. F. 2009). Thus, in yet another embodiment, reduction of cortisol is useful as an aid to weight loss. Accordingly, also provided herein is a method of treating obesity or reducing fat comprising administering one or more compounds of the formula (I) to an animal in need thereof. Also provided herein is use of one or more compounds of the formula (I) for treating obesity or reducing fat in an animal in need thereof. Further provided is use of one or more compounds of the formula (I) in the preparation of a medicament for treating obesity or reducing fat in an animal in need thereof. Even further provided is one or more compounds of the formula (I) for use in treating obesity or reducing fat in an animal in need thereof.

High cortisol levels have been shown to result in suppression of the manufacturing of DHEA, which is the precursor of reproductive hormones, including estrogen, progesterone and testosterone. Animals under stress have a more difficult time becoming pregnant and methods for reducing cortisol would be useful to aid in reproductive efficiency. Accordingly, in a further embodiment, also provided herein is a method of improving reproductive efficiency comprising administering one or more compounds of the formula (I) to an animal in need thereof. Also provided herein is use of one or more compounds of the formula (I) for improving reproductive efficiency in an animal in need thereof. Further provided is use of one or more compounds of the formula (I) in the preparation of a medicament for improving reproductive efficiency in an animal in need thereof. Even further provided is one or more compounds of the formula (I) for use in improving reproductive efficiency in an animal in need thereof. The term “improving reproductive efficiency” as used herein refers to increasing the likelihood of conceiving or maintaining a pregnancy compared to an untreated control.

High cortisol and low DHEA levels are also implicated in metabolic disorders, including, without limitation, Crohn's Disease and Ulcerative Colitis. Reduction of cortisol is useful for treating such metabolic disorders, including without limitation, Crohn's Disease Ulcerative Colitis, by lowering cortisol, increasing DHEA and reducing inflammation. Accordingly, in yet a further embodiment, also provided herein is a method of treating a metabolic disorder comprising administering one or more compounds of the formula (I) to an animal in need thereof. Also provided herein is use of one or more compounds of the formula (I) for treating a metabolic disorder in an animal in need thereof. Further provided is use of one or more compounds of the formula (I) in the preparation of a medicament for treating a metabolic disorder in an animal in need thereof. Even further provided is one or more compounds of the formula (I) for use in treating a metabolic disorder in an animal in need thereof.

Cortisol levels are further implicated in hypertension and thus, in another embodiment, methods and uses for reducing cortisol are useful in treating hypertension. Accordingly, also provided herein is a method of treating hypertension comprising administering one or more compounds of the formula (I) to an animal in need thereof. Also provided herein is use of one or more compounds of the formula (I) for treating hypertension in an animal in need thereof. Further provided is use of one or more compounds of the formula (I) in the preparation of a medicament for treating hypertension in an animal in need thereof. Even further is one or more compounds of the formula (I) for use in treating hypertension in an animal in need thereof.

Reduction of cortisol is useful in reducing high blood sugar, including, without limitation, for treating hyperglycemia, insulin resistance and Type 2 diabetes. Accordingly, in yet another embodiment, also provided herein is a method of reducing high blood sugar comprising administering one or more compounds of the formula (I) to an animal in need thereof. Also provided herein is use of one or more compounds of the formula (I) for reducing high blood sugar in an animal in need thereof. Further provided is use of one or more compounds of the formula (I) in the preparation of a medicament for reducing high blood sugar in an animal in need thereof. Even further is one or more compounds of the formula (I) for use in reducing high blood sugar in an animal in need thereof. The term “reducing high blood sugar” as used herein refers to lowering blood sugar into the normal range in an animal that suffers from high blood sugar, including, without limitation, an animal that has hyperglycemia, insulin resistance and/or Type 2 diabetes.

Reduction of weight is useful for ameliorating number of conditions related to but not limited to general health, obesity, metabolic syndrome diseases and diabetes. Accordingly, in yet another embodiment, also provided herein is a method of reducing weight comprising administering one or more compounds of the formula (I) to an animal in need thereof. Also provided herein is use of one or more compounds of the formula (I) for reducing weight in an animal in need thereof. Further provided is use of one or more compounds of the formula (I) in the preparation of a medicament for reducing weight in an animal in need thereof. Even further is one or more compounds of the formula (I) for use in reducing weight in an animal in need thereof. The term “reducing weight” as used herein refers to lowering the weight of the animal into the normal range for said an animal.

The present disclosure also includes a method for treating post-traumatic stress disorder. Accordingly, in one embodiment, provided herein is a method for the treatment of post-traumatic stress disorder comprising administering one or more compounds of the formula (I) to an animal in need thereof. Also provided herein is use of one or more compounds of the formula (I) for the treatment of post-traumatic stress disorder in an animal in need thereof. Further provided is use of one or more compounds of the formula (I) in the preparation of a medicament for the treatment of post-traumatic stress disorder in an animal in need thereof.

The term “treatment or treating” as used herein means an approach for obtaining beneficial or desired results, including clinical results. Beneficial or desired results can include, but are not limited to, alleviation or amelioration of one or more symptoms or conditions, diminishment of extent of condition (stress), stabilized (i.e. not worsening) state of the condition (stress) and preventing the condition (stress). Thus, treatment can be administered while symptoms are present or prophylactically in advance of anticipated development of symptoms in animals at heightened risk of developing symptoms, for example, of a condition associated with high levels of cortisol.

The reduction of cortisol is also known to make cancer and immune therapies work more effectively. Accordingly, in another embodiment, there is provided a method of treating cancer or an immune condition comprising administering a cancer or immune therapeutic contemporaneously with one or more compounds of the formula (I). Also provided herein is use of a cancer or immune therapeutic and one or more compounds of the formula (I) for treating cancer or an immune condition in an animal in need thereof. Further provided is use of a cancer or immune therapeutic and one or more compounds of the formula (I) in the preparation of a medicament for treating cancer or an immune condition in an animal in need thereof. Even further is a cancer or immune therapeutic and one or more compounds of the formula (I) for use in treating cancer or an immune condition in an animal in need thereof.

Reference to the compounds of the formula (I) above includes all of the compounds of the present disclosure including compounds of the formula (Ia), (Ib), (Ic), (Id), (Ie), (If) and (Ig).

The disclosure therefore relates to administration of a therapeutically effective amount of a compound of the formula (I) for reducing glucocorticoids to an animal in need thereof, the compound of the formula (I) comprising

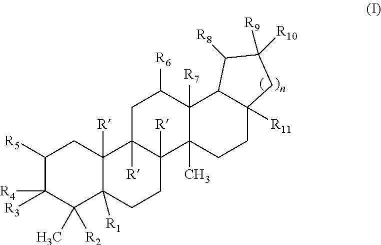

wherein
R′ are each independently or simultaneously H or CH₃;

### R₁ is H or CH₃;

### R₂ is H, CH₃ or CH₂OH;

R₃ is OH, —OCH₃, —OC(O)—(C₁₋₆)alkyl, —OC(O)—(C₆₋₁₀)aryl, —OC(O)—(C₆₋₁₀)heteroaryl, —NC(O)—(C₁₋₆)alkyl, —NC(O)-benzyl;
R₄ is H, —CH₃, (C₆-C₁₀)aryl or (C₆-C₁₀)heteroaryl; or
R₃ and R₄ are joined together to form C═O;
R₅ is H, OH, (C₁₋₆)alkyl or (C₂₋₆)alkenyl;
R₆ and R₇ are independently or simultaneously H or CH₃, or R₆ and R₇ are joined together to form a double bond (C═C);
R₈ is H, (C₁₋₆)alkyl, (C₂₋₆)alkenyl, (C₁₋₆)alkanol, (C₂₋₆)alkenol, —C(O)—(C₁₋₆)alkyl, —C(O)OH, —C(O)O—(C₁-C₆)alkyl, —C(O)—CH₂CH₂—(C₆-C₁₀)aryl, —C(O)—CH₂CH₂—(C₆-C₁₀)heteroaryl, —C(O)—(C₂-C₆)alkenyl, —C(O)—CH₂—CH═CH—(C₆-C₁₀)aryl, —C(O)—CH₂—CH═CH—(C₆-C₁₀)heteroaryl

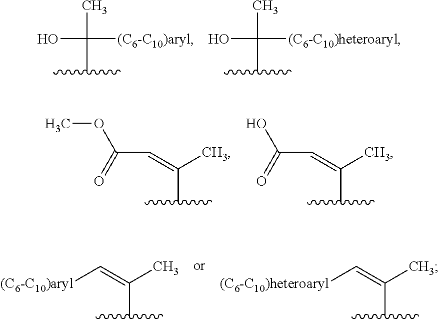

R₉ and R₁₀ are independently or simultaneously H or CH₃;
R₁₁ is CH₃, CH₂OH, CH₂OCOCH₃, —CH(O), —C(O)ORₐ, —C(O)O—(C₁₋₆)alkyl-C(O)O—R_{b}, C(O)—NH—(C₁₋₆)alkyl-C(O)O—R_{b}; or —CH═CH—C(O)O—R_{b};
wherein Rₐ is H, (C₁₋₁₀)alkyl, (C₁₋₆)alkenyl, —CH₂—(C₆-C₁₀)-aryl, or —CH₂—(C₆-C₁₀)-heteroaryl
R_{b} is H or (C₁₋₆)alkyl;
n is the integer 1 or 2; and
the aryl groups are optionally substituted with one to five substituents selected from halo, OH or (C₁₋₃)alkyl,
or a pharmaceutically acceptable salt or solvate thereof, and any stereoisomer (entantiomer, diastereomer) thereof,
with the proviso that the compound of the formula (I) is not

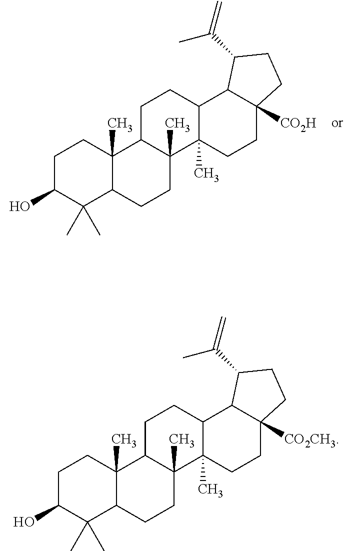

In another embodiment of the disclosure, the compound of the formula (I) comprises a compound of the formula (Ia):

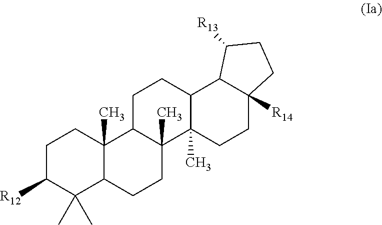

wherein

### R₁₂ is OH, —O—CH₃ or —OC(O)CH₃;

R₁₃ is (C₁-C₆)alkyl, (C₂-C₆)alkenyl, (C₁-C₆)-alkanol, (C₂-C₆)-alkenol, —C(O)—(C₁₋₆)alkyl, —C(O)OH, —C(O)O—(C₁-C₆)alkyl, —C(O)—CH₂CH₂—(C₆-C₁₀)aryl, —C(O)—CH₂CH₂—(C₆-C₁₀)heteroaryl, —C(O)—(C₂-C₆)alkenyl, —C(O)—CH₂—CH═CH—(C₆-C₁₀)aryl, —C(O)—CH₂—CH═CH—(C₆-C₁₀)heteroaryl,

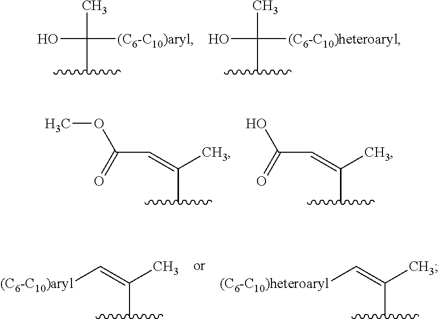

R₁₄ is CH₃, CH₂OH, CH₂OC(O)CH₃)—C(O)ORₐ or —C(O)O—(C₁₋₆)alkyl-C(O)O—R_{b}; and
wherein Rₐ is H, (C₁₋₁₀)alkyl, (C₁₋₆)alkenyl, —CH₂—(C₆-C₁₀)-aryl, or CH₂—(C₆-C₁₀)-heteroaryl;
R_{b} is H or (C₁₋₆)alkyl.

In another embodiment, R₁₃ is —CO₂H, —CO₂CH₃,

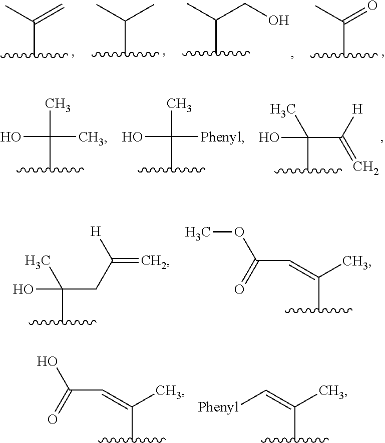

—C(O)—CH₂CH═CH₂, —C(O)—CH═CH—CH₃, —C(O)—CH₂CH₂-phenyl, or —C(O)—CH═CH-phenyl; and
R₁₄ is CH₃, CH2OH, CH₂OC(O)CH—CO₂(benzyl), —CO₂—CH₂—CO₂CH₂CH₃, —CO₂H or —CO₂CH₃.

In a further embodiment, R₁₃ is —CO₂H, —CO₂CH₃,

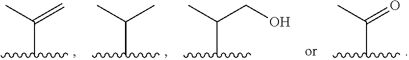

Compounds of the formula (Ia) include

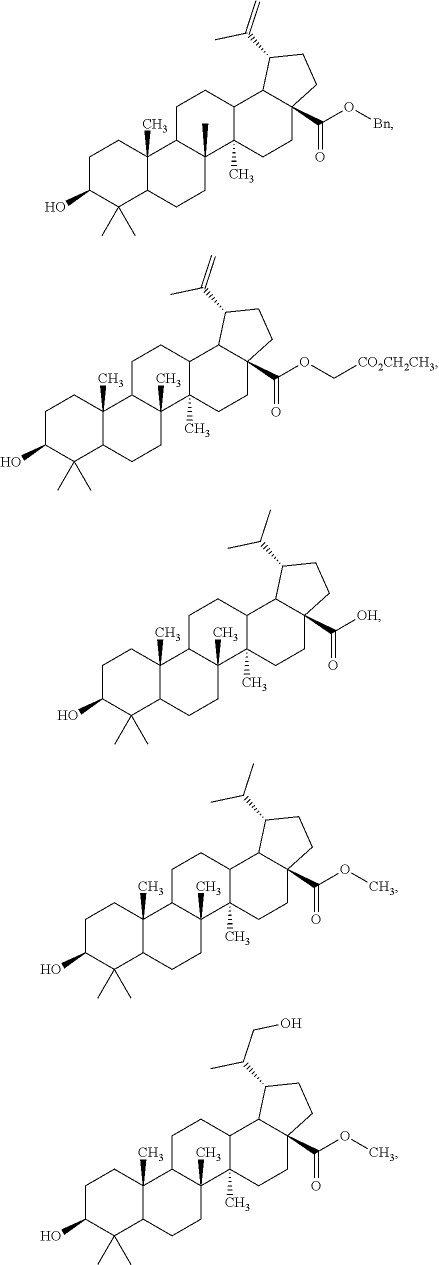

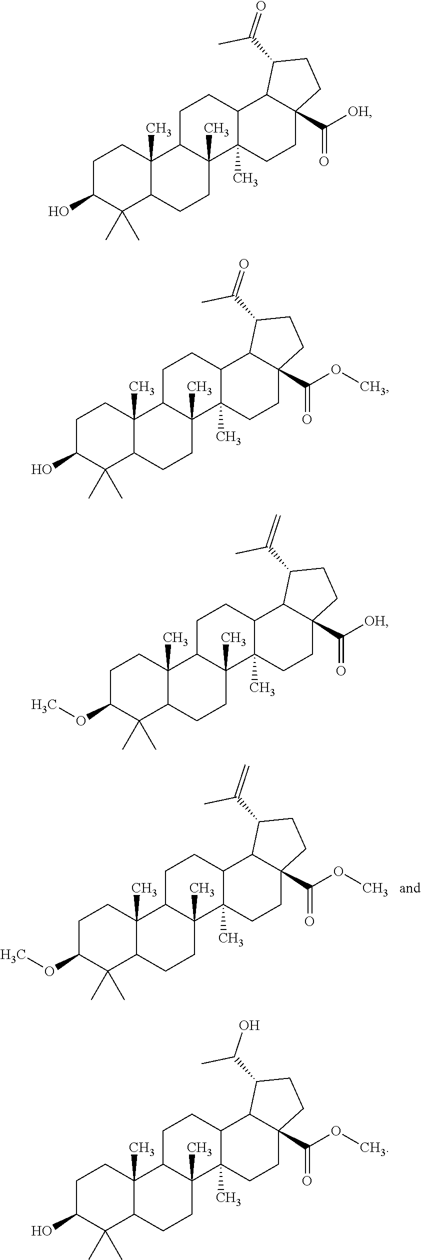

Further compounds of the formula (Ia) include

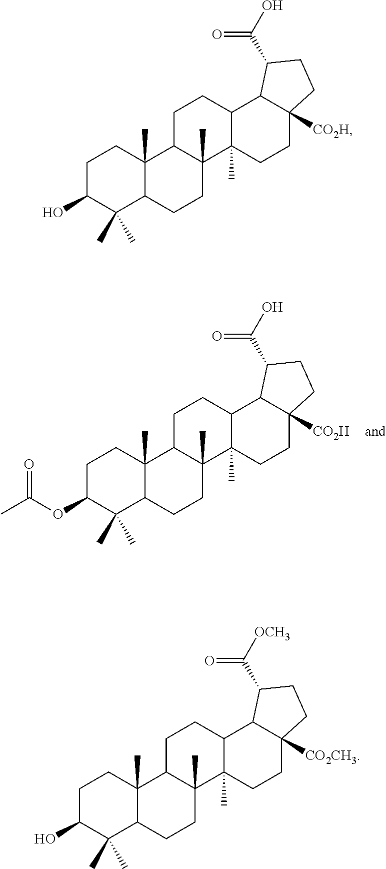

Additional compounds of the formula (Ia) include

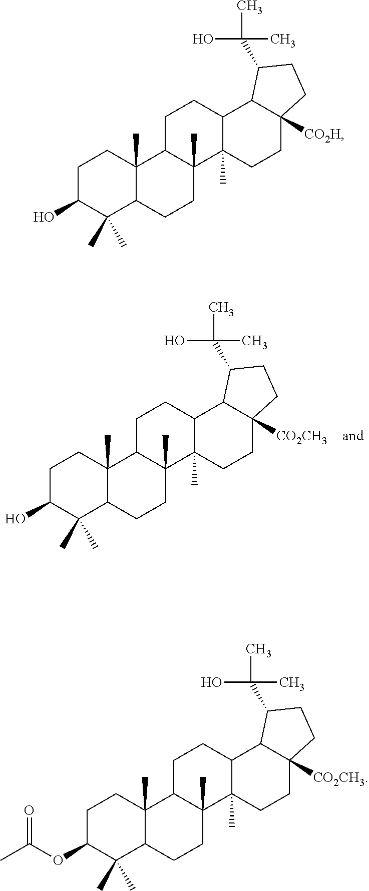

Further compounds of the formula (Ia) include

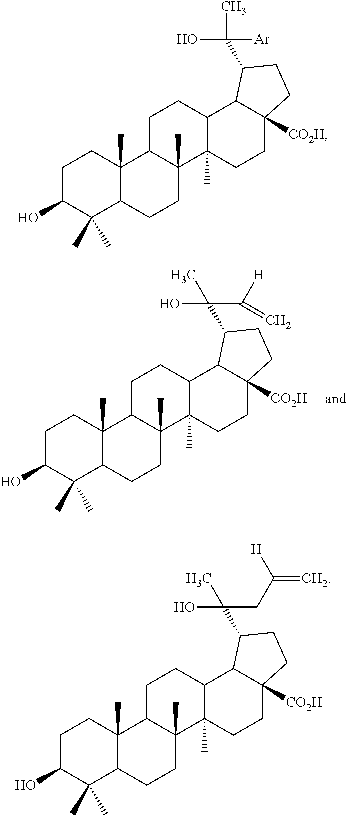

Additional compounds of the formula (Ia) include

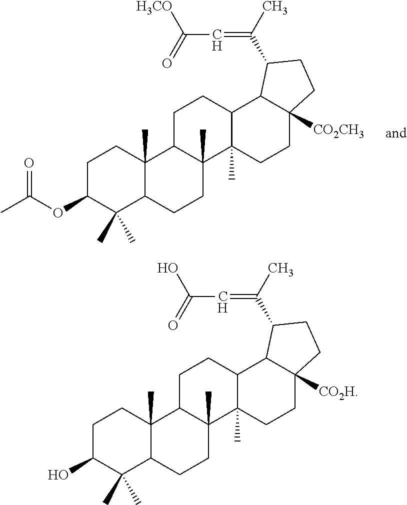

In another embodiment of the disclosure, the compound of the formula (I) comprises a compound of the formula (Ib):

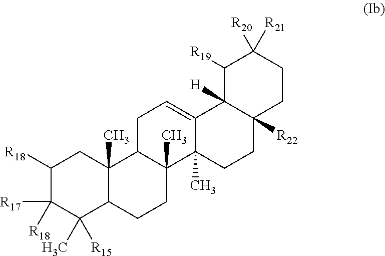

wherein

### R₁₅ is CH₃, or CH₂OH or CH₂OC(O)CH₃;

### R₁₆ is OH or —OC(O)CH₃;

### R₁₇ is H; or

R₁₆ and R₁₇ are joined together to form C═O;

### R₁₈ is H or OH;

R₁₉-R₂₁ are each simultaneously or independently H or CH₃; and

### R₂₂ is CH₃, CH₂OH, CH₂OC(O)CH₃, —C(O)OH or —C(O)OCH₃.

Compounds of the formula (Ib) include

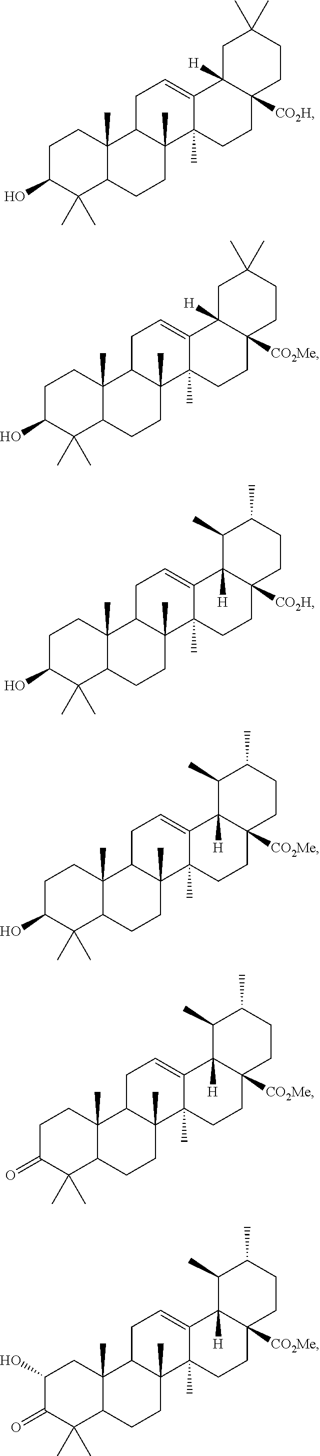

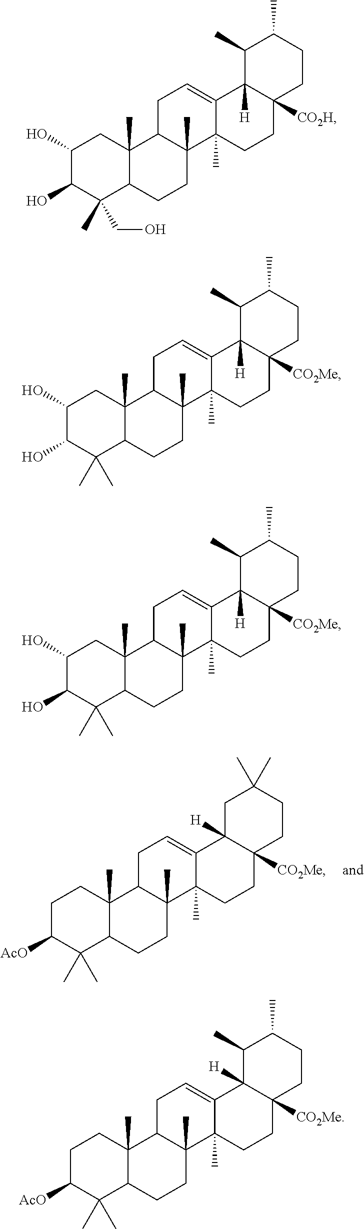

Further compounds of the formula (Ib) include

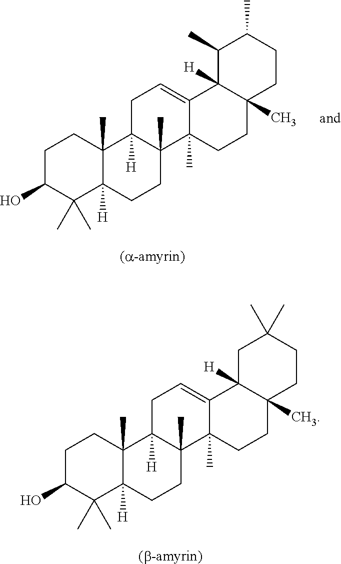

In another embodiment, the compound of the formula (I) comprises a compound of the formula (Ic)

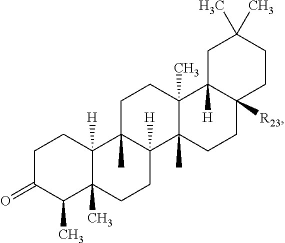

wherein R₂₃ is CH₃, CH₂OH, —C(O)OH or —C(O)OCH₃.

Compounds of the formula (Ic) include

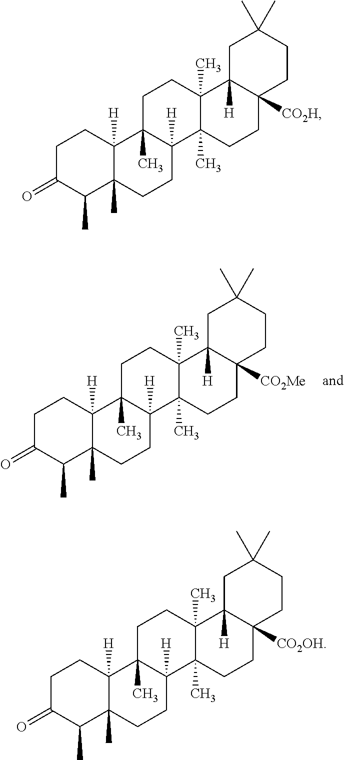

In another embodiment, the compound of the formula (I) comprises a compound of the formula (Id)

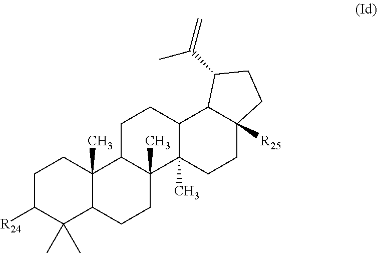

wherein
R₂₄ is —OC(O)—(C₁₋₆)alkyl, —OC(O)—(C₆₋₁₀)aryl, —OC(O)—(C₆₋₁₀)heteroaryl, —NC(O)—(C₁₋₆)alkyl, —NC(O)-benzyl;

### R₂₅ is —C(O)ORₐ;

wherein Rₐ is H or (C₁₋₁₀)alkyl,
wherein the alkyl, aryl or heteroaryl groups are optionally substituted with one to five substituents selected from halo, OH or (C₁₋₃)alkyl.

In another embodiment,

### R₂₄ is —OC(O)—CH₃, —OC(O)-phenyl, —NC(O)—CH₃, —NC(O)-benzyl;

### R₂₅ is —C(O)OH or —C(O)OMe; and

wherein the phenyl group is optionally substituted with halo, such as fluoro, chloro and bromo.

Compounds of the formula (Id) include

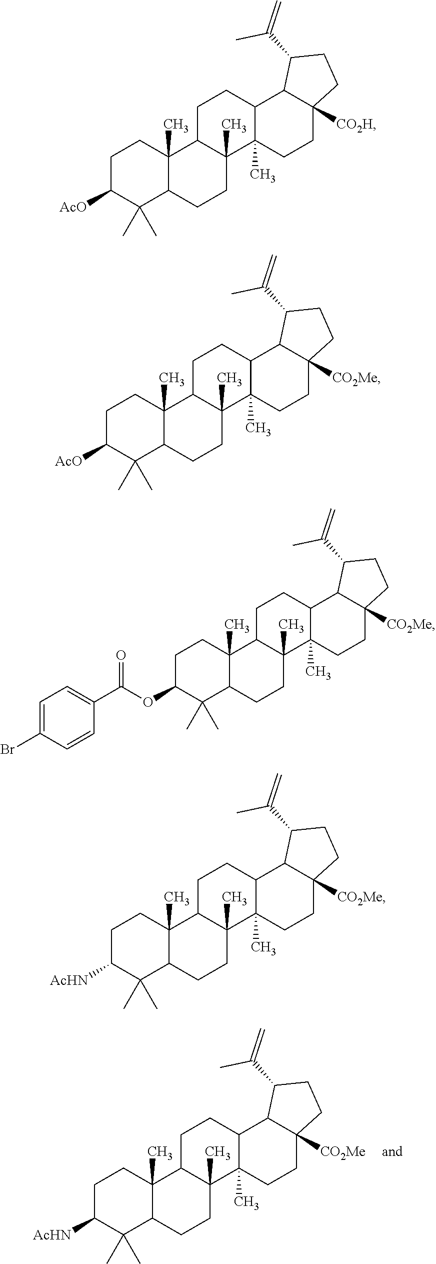

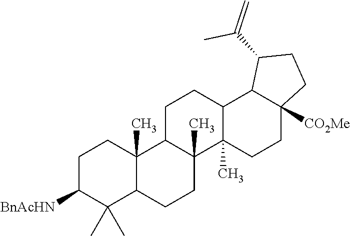

In another embodiment, the compound of the formula (I) comprises a compound of the formula (Ie)

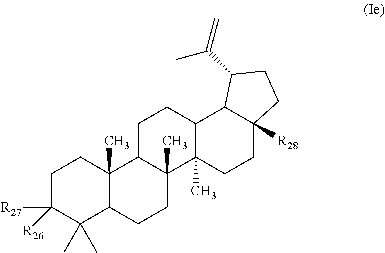

wherein

### R₂₆ is OH;

### R₂₇ is H; or

R₂₆ and R₂₇ are joined together to form C═O;
R₂₈ is CH₃, CH₂OH, CH₂OC(O)CH₃, CH(O), —C(O)ORₐ, —C(O)O—(C₁₋₆)alkyl-C(O)O—R_{b}, C(O)—NH—(C₁₋₆)alkyl-C(O)O—R_{b}, or —CH═CH—C(O)O—R_{b};
wherein Rₐ is (C₁₋₁₀)alkyl or (C₁₋₆)alkenyl,
R_{b} or H, (C₁₋₆)alkyl;
wherein the alkyl groups are optionally substituted with one to five substituents selected from halo, OH or (C₁₋₃)alkyl.

In another embodiment, R₂₈ is CH(O), —C(O)O(CH₂)₆CH₃, —CO₂—CH₂—CH═CH₂, —C(O)—NH—CH₂—COOH, —CH═CH—CO₂—CH₂CH₃, or —CH═CH—CO₂H.

Compounds of the formula (Ie) include

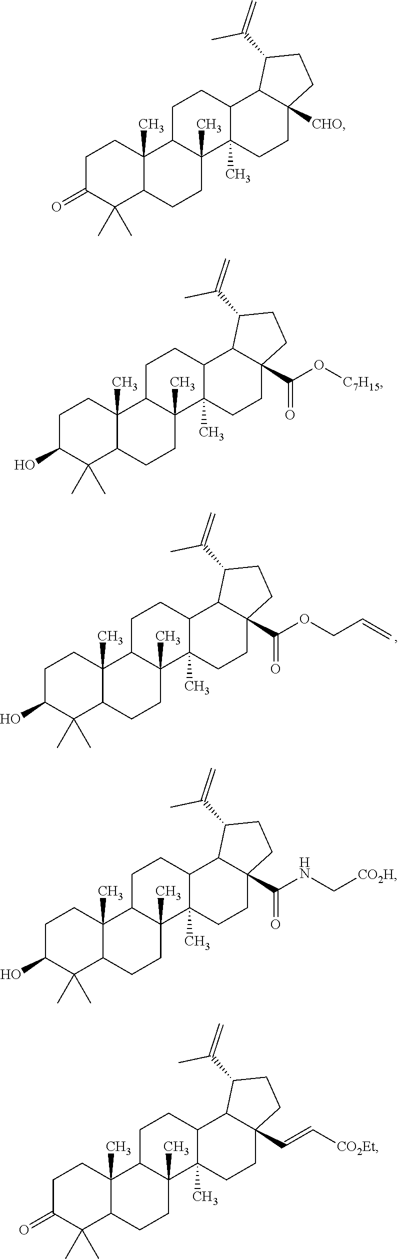

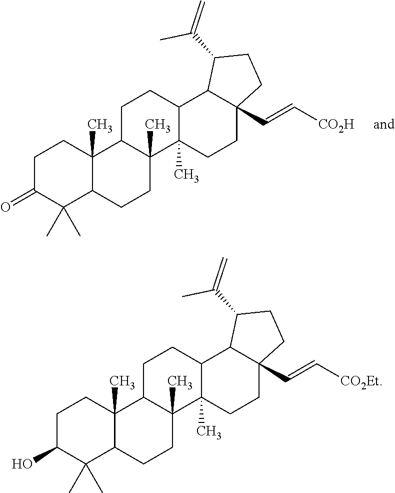

In another embodiment, the compound of the formula (I) comprises a compound of the formula (If)

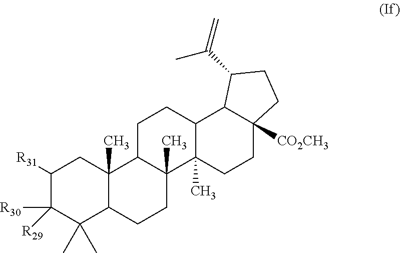

wherein

### R₂₉ is OH;

### R₃₀ is H; or

R₂₉ and R₃₀ are joined together to form C═O; and
R₃₁ is OH or (C₂₋₆)alkenyl.

Compounds of the formula (If) include

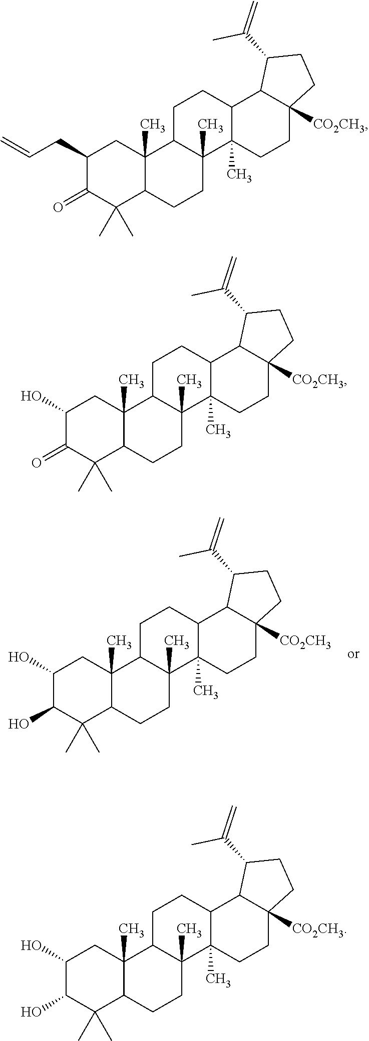

In another embodiment, the compound of the formula (I) comprises a compound of the formula (Ig)

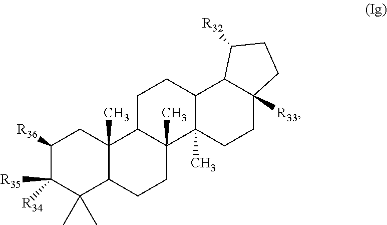

wherein
R₃₂ is —C(O)—(C₁₋₆)-alkanol, —(C₁₋₆)-alkanol, —(C₁₋₆)-dialkanol, or —C(O)—(C₁₋₆)-alkyl;

### R₃₃ is —CO₂H, —C(O)N(R_{b})₂, or —C(O)NR_{b}—CH₂CO₂H;

### R₃₄ is H;

### R₃₅ is OH; or

R₃₄ and R₃₅ are joined together to form C═O;

### R₃₆ is H or OH;

with the proviso that when R₃₂ is —(C₁₋₆)-alkanol or —C(O)—(C₁₋₆)-alkyl, R₃₆ is OH.

In another embodiment, R₃₂ is —C(O)—CH₂OH, —CH(OH)CH₃, —CH(OH)CH₂OH, or —C(O)CH₃.

In another embodiment, R₃₃ is —COOH, —CONH₂, or —C(O)NH—CH₂CO₂H.

In another embodiment, the compound of the formula (Ig) is

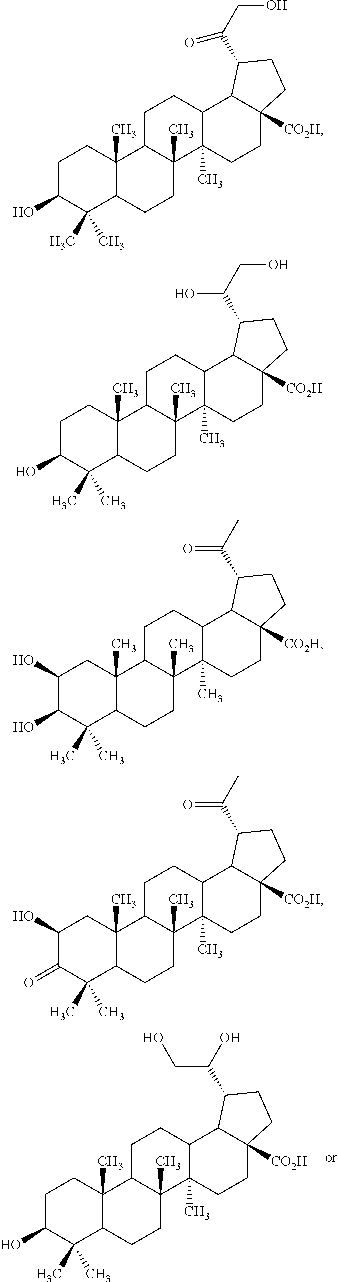

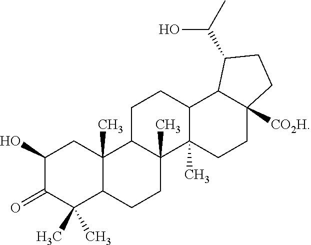

### (II) Pharmaceutical Compositions for Reducing Glucocorticoids

The present disclosure also relates to pharmaceutical compositions comprising a compound of the formula (I), which includes compounds of the formulae (Ia, Ib, Ic, Id, Ie, If and Ig), and pharmaceutically acceptable carriers and/or excipients for reducing glucocorticoids, wherein the composition is administered to an animal in need thereof.

The pharmaceutical compositions can be prepared by per se known methods for the preparation of pharmaceutically acceptable compositions which can be administered to patients, and such that an effective quantity of the active ingredients is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 2003—20^{th} Edition) and in The United States Pharmacopeia: The National Formulary (USP 24 NF19) published in 1999).

In an embodiment, the carrier is a binder, such as but not limited to microcrystalline cellulose, and other binders commonly known to the person skilled in the art.

The compositions disclosed herein may be administered in any manner, such as, intranasally, intraocularly, sublingually, orally, parenterally, intravenously, topically, intraperitoneally, or rectally.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The pharmaceutical composition may be supplied, for example but not by way of limitation, as a lyophilized powder which is reconstituted with sterile water or saline prior to administration to the patient.

Suitable carriers include, without limitation, fillers, such as sugars, for example lactose, saccharose, mannitol or sorbitol, cellulose preparations and/or calcium phosphates, for example tricalcium phosphate or calcium hydrogen phosphate, also binders, such as starch pastes, using, for example, corn, wheat, rice or potato starch, gelatin, gum tragacanth, methyl-cellulose and/or polyvinylpyrrolidone, and if desired, disintegrators, such as the above-mentioned starches, also carboxymethyl starch, cross-linked polyvinylpyrrolidone, agar or alginic acid or a salt thereof, such as sodium alginate, vegetable oils, gums, methyl cellulose, PVP, cyclodextrose, maltodextrose, flavoring agents, flavored powders, smoothies, gels etc.

Suitable excipients include, without limitation, flow conditioners and lubricants, for example, silicic acid, talc, stearic acid or salts thereof, such as magnesium or calcium stearate, and/or polyethylene glycol. Dragee cores are provided with suitable coatings that may be resistant to gastric juices, there being used, inter alia, concentrated sugar solutions which may contain gum Arabic, talc polyvinylpyrrolidone, polyethylene glycol and/or titanium dioxide, coating solutions in suitable organic solvents or solvent mixtures, or, for the preparation of enteric coatings, solutions of suitable cellulose preparations, such as acetylcellulose phthalate or hydroxypropylmethylcellulose phthalate or microcrystalline cellulose. Colourings or pigments may be added to the tablets or dragee coatings, for example for identification purposes or to indicate different doses of active ingredient.

Further orally administrable pharmaceutical compositions include dry-filled capsules consisting of gelatin, and also soft sealed capsules consisting of gelatin and a plasticizer, such as glycerol or sorbitol. The dry-filled capsules may contain the active ingredients in the form of granules, for example, in admixture with fillers, such as lactose, binders, such as starches, and/or glidants, such as talc or magnesium stearate, and optionally stabilizers. In soft capsules, the active ingredients may be dissolved or suspended in suitable liquids such as fatty oils, paraffin oil or liquid polyethylene glycols, to which stabilizers may be added.

Rectally administrable pharmaceutical compositions, for example, suppositories that comprise a combination of the active ingredients and a suppository base are also provided. Suitable as suppository bases are, for example, natural or synthetic triglycerides, paraffin hydrocarbons, polyethylene glycols and higher alkanols. It is also possible to use gelatin rectal capsules that comprise a combination of the active ingredients and base material. Suitable base materials are, for example, liquid triglycerides, polyethylene glycols and paraffin hydrocarbons. Administration of an “effective amount” of the compositions in the methods and uses of the present disclosure is defined as an amount effective, at dosages and for periods of time necessary to achieve the desired result.

The compositions disclosed herein may also be administered parenterally. Solutions can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2000—20th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases, the form must be sterile and must be fluid to the extent that easy syringability exists.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine.

The dosage of the compositions disclosed herein can vary depending on many factors such as the pharmacodynamic properties of the active ingredients, the mode of administration, the age, health and weight of the recipient, the nature and extent of the symptoms, the frequency of the treatment and the type of concurrent treatment, if any, and the clearance rate of the composition in the animal to be treated. One of skill in the art can determine the appropriate dosage based on the above factors. The compositions disclosed herein may be administered initially in a suitable dosage that may be adjusted as required, depending on the clinical response. As a representative example suitable for animals, oral dosages of a composition will range between about 0.1 mg/kg of Body Wt per day to about 25.0 mg/kg (Body Wt per day for an adult), more suitably about 1 mg/kg of body weight per day to about 2.5 mg/kg of body weight per day. When formulated for oral administration to animals, the compositions for example, are suitably in the form of tablets containing 0.1, 1, 5, 10, 20, 40, 80, 100, 200, 300, 400 or 800 mg of active ingredient per tablet. The compositions may be administered in a single daily dose or the total daily dose may be divided into two, three of four doses.

The mode of administration (e.g. in vivo by injection) will also impact the dosage regimen. For example, if the compositions are to be administered transdermally, using, for example, those forms of transdermal skin patches that are well known to those skilled in the art, the dosage administration will be continuous rather than intermittent throughout the dosage range.

### (III) Compounds of the Formula (I)

The present disclosure also includes compounds of the formula (I) as described above.

In particular, the disclosure includes the following compounds

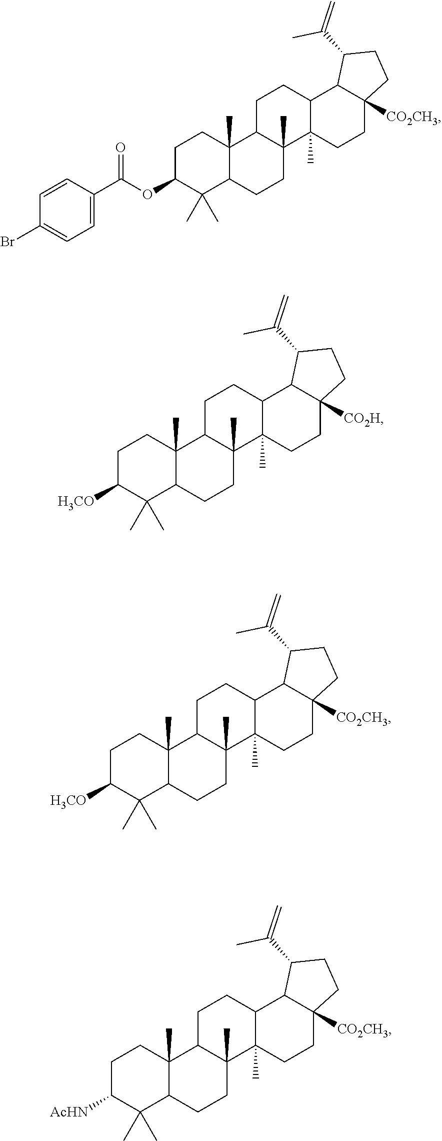

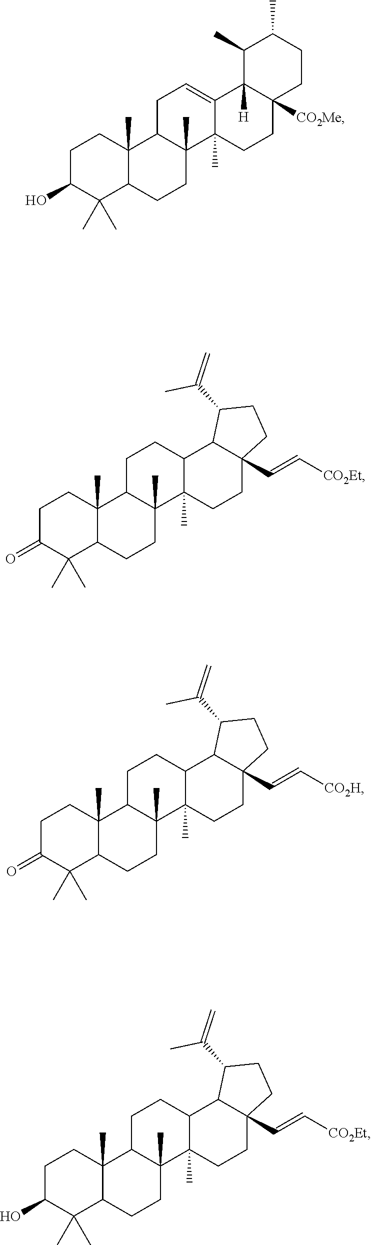

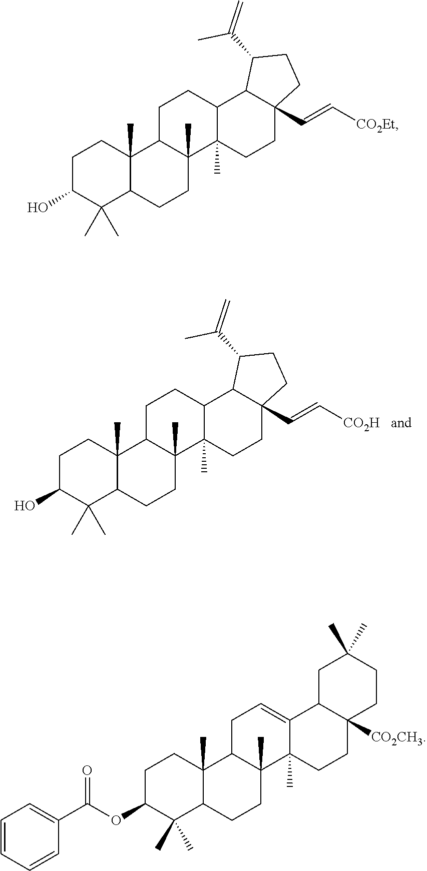

Further compounds of the formula (I) include

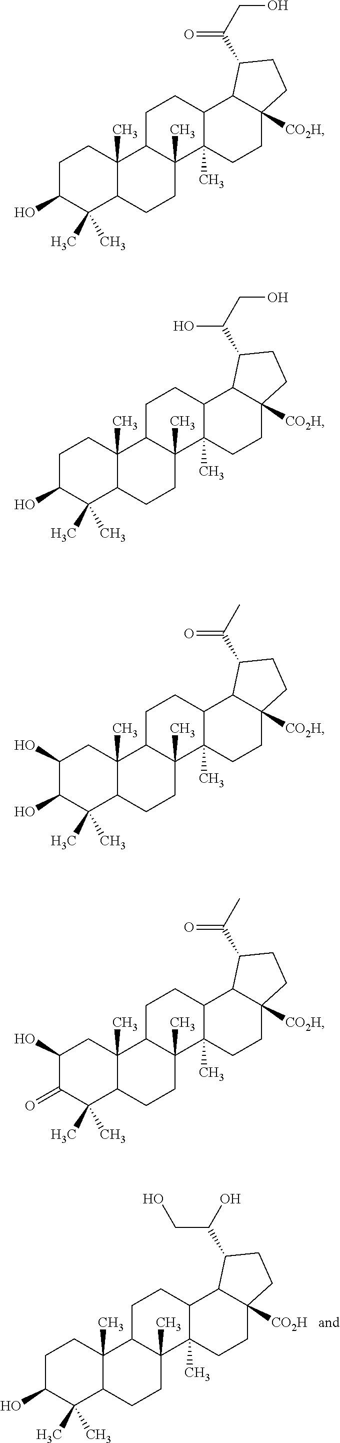

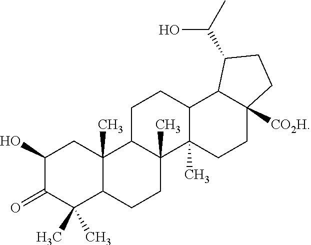

### (IV) Synergistic Combinations

The present disclosure also includes synergistic combinations of compounds for reducing glucocorticoids and/or treating anxiety in an animal in need thereof. Accordingly, in one embodiment, the present disclosure includes pharmaceutical compositions comprising a compound of the formula (IIa):

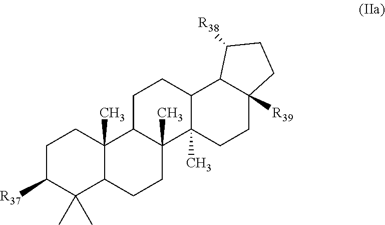

wherein

### R₃₇ is OH, —O—CH₃ or —OC(O)CH₃;

R₃₈ is (C₁-C₆)alkyl, (C₂-C₆)alkenyl, (C₁-C₆)-alkanol, (C₂-C₆)-alkenol, —C(O)—(C₁₋₆)alkyl, —C(O)OH, —C(O)O—(C₁-C₆)alkyl, —C(O)—CH₂CH₂—(C₆-C₁₀)aryl, —C(O)—CH₂CH₂—(C₆-C₁₀)heteroaryl, —C(O)—(C₂-C₆)alkenyl, —C(O)—CH₂—CH═CH—(C₅-C₁₀)aryl, —C(O)—CH₂—CH═CH—(C₆-C₁₀)heteroaryl,

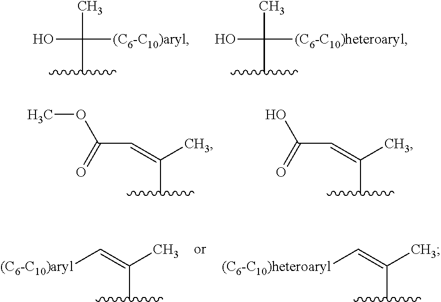

R₃₉ is CH₃, CH₂OH, CH₂OC(O)CH₃)—C(O)ORₐ or —C(O)O—(C₁₋₆)alkyl-C(O)O—R_{b}; and
wherein Rₐ is H, (C₁₋₁₀)alkyl, (C₁₋₆)alkenyl, —CH₂—(C₆-C₁₃)-aryl, or CH₂—(C₆-C₁₀)-heteroaryl;
R_{b} is H or (C₁₋₆)alkyl;
or a pharmaceutically acceptable salt or solvate thereof, and any stereoisomer (entantiomer, diastereomer) thereof,
and
a compound of the formula (IIb):

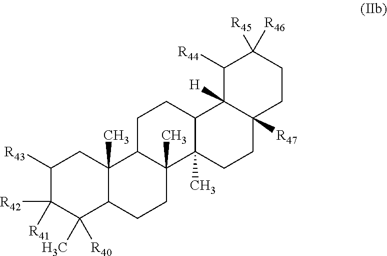

wherein

### R₄₀ is CH₃, or CH₂OH or CH₂OC(O)CH₃;

### R₄₁ is OH or —OC(O)CH₃;

### R₄₂ is H; or

R₄₁ and R₄₂ are joined together to form C═O;

### R₄₃ is H or OH;

R₄₄-R₄₆ are each simultaneously or independently H or CH₃; and

### R₄₇ is CH₃, CH₂OH, CH₂OC(O)CH₃, —C(O)OH or —C(O)OCH₃,

or a pharmaceutically acceptable salt or solvate thereof, and any stereoisomer (entantiomer, diastereomer) thereof,

In one embodiment, R₃₇ is OH.

In another embodiment, R₃₈ is —CO₂H, —CO₂CH₃,

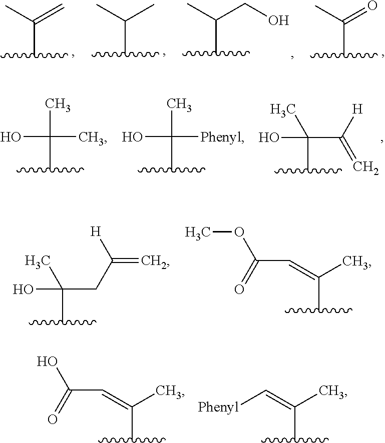

—C(O)—CH₂CH═CH₂, —C(O)—CH═CH—CH₃, —C(O)—CH₂CH₂-phenyl, or —C(O)—CH═CH-phenyl; and
R₃₉ is CH₃, CH2OH, CH₂OC(O)CH—CO₂(benzyl), —CO₂—CH₂—CO₂CH₂CH₃, —CO₂H or —CO₂CH₃.

In a further embodiment, R₃₆ is —CO₂H, —CO₂CH₃,

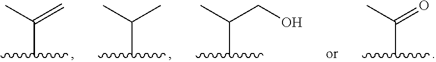

Compounds of the formula (IIa) include

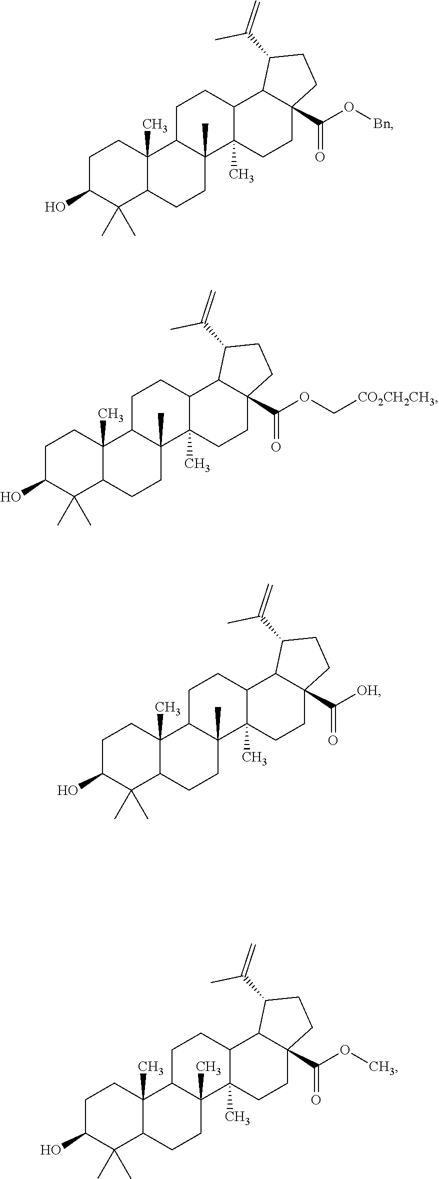

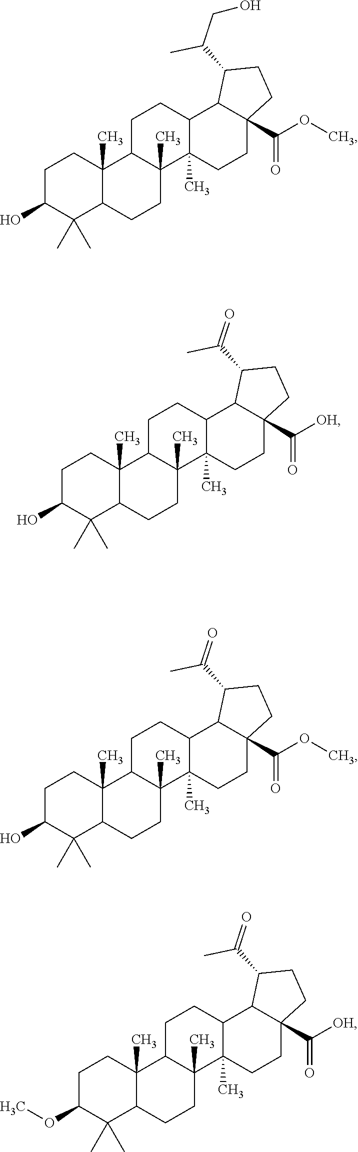

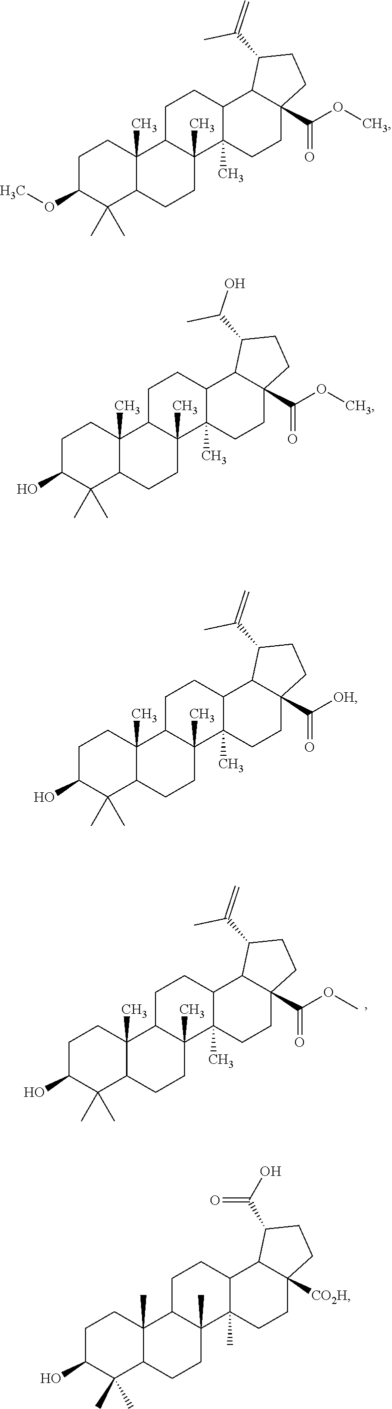

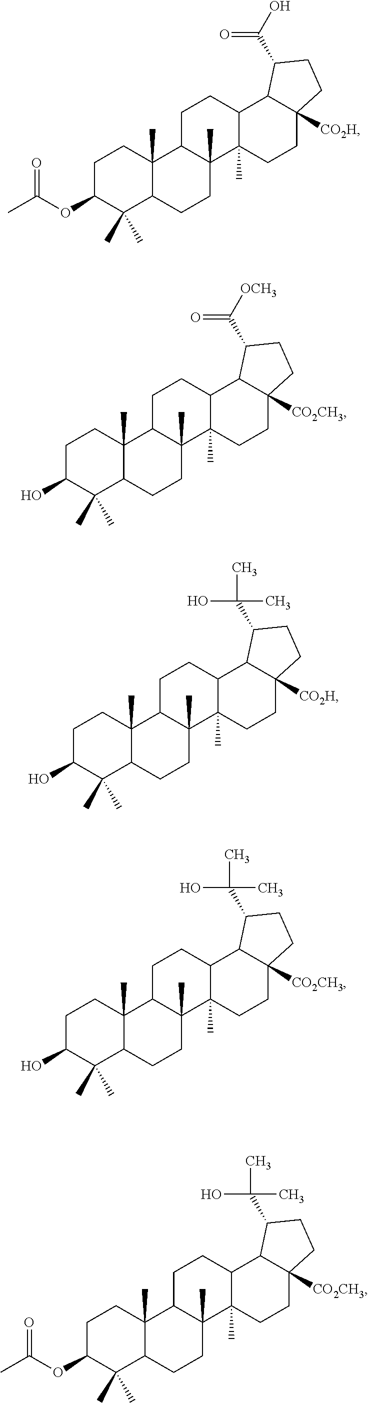

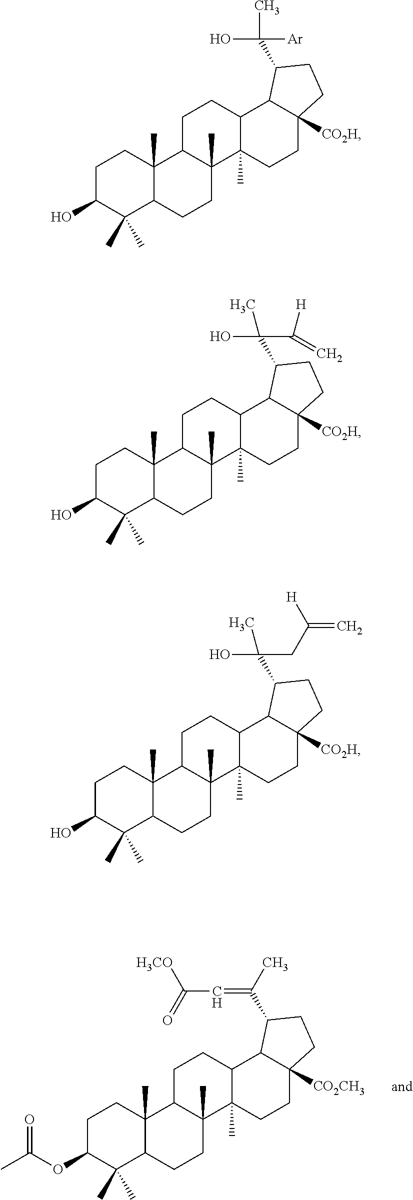

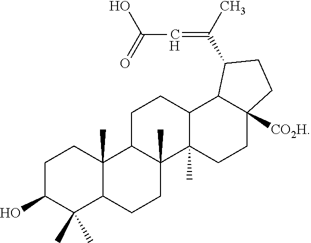

Compounds of the formula (IIb) include

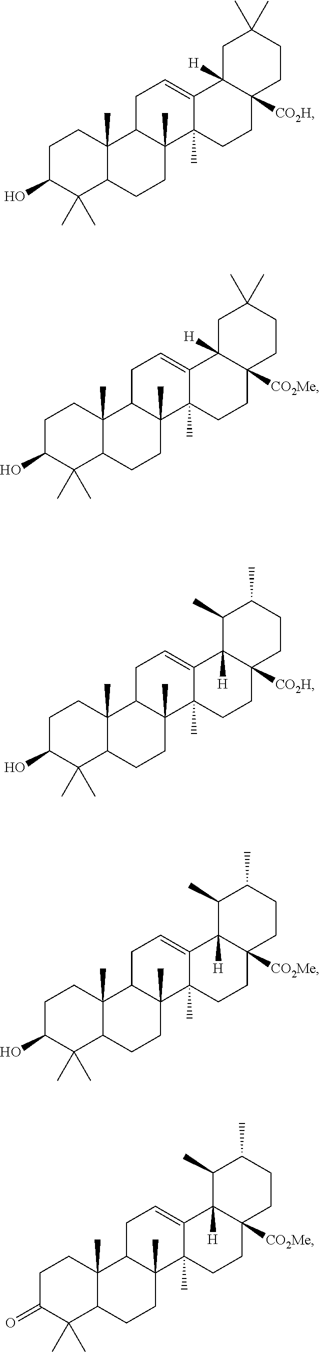

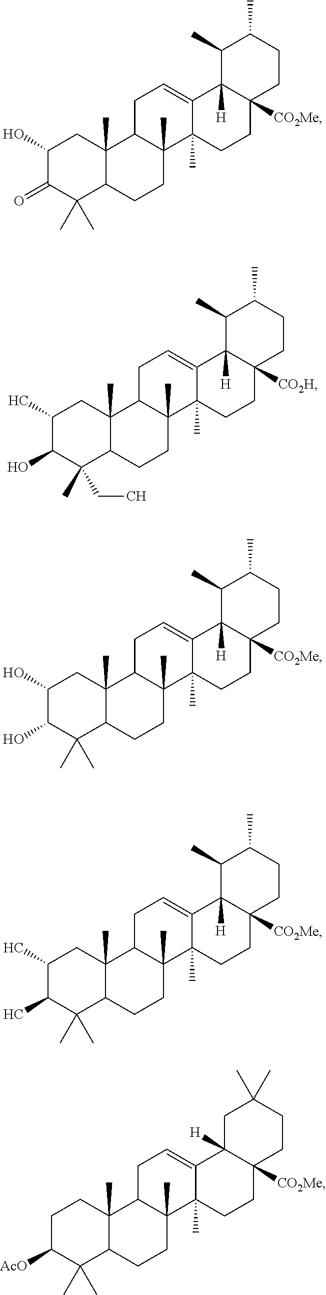

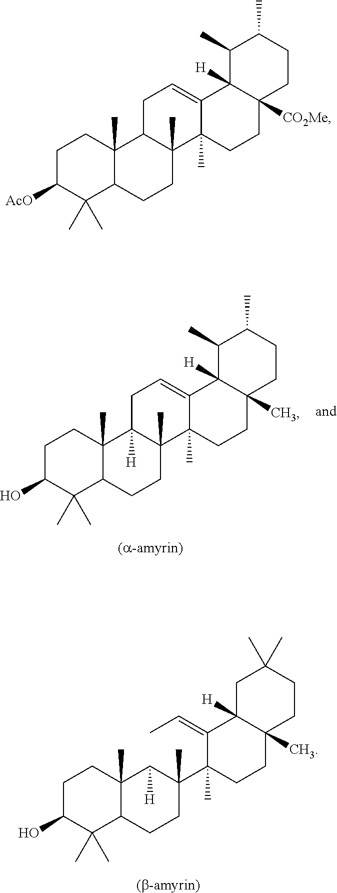

In one embodiment, the synergistic combination comprises a pharmaceutical composition comprising at least one compound of the formula (IIa) and at least one compound of the formula (IIb). In one embodiment, the compound of the formula (IIa) is

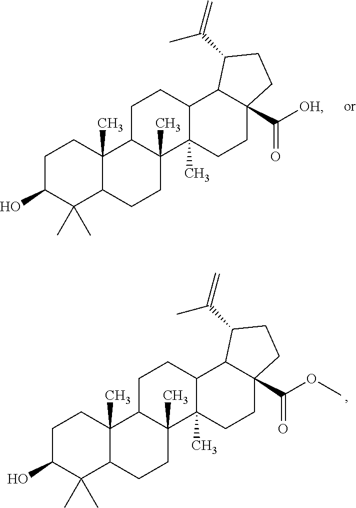

and the compound of the formula (IIb) is

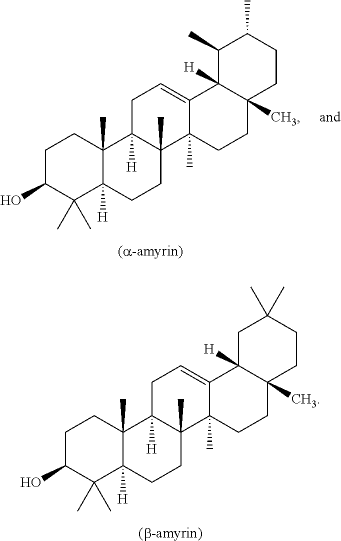

In one embodiment, the synergistic pharmaceutical composition comprises

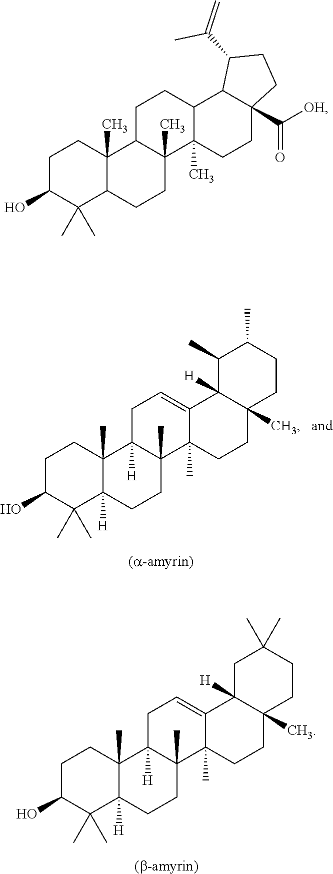

In another embodiment, the synergistic pharmaceutical composition is useful for the reduction of glucocorticoid, such as cortisol/corticosterone, and useful for the treatment of maintaining bone density, maintaining and improving the immune system, treating Cushing's syndrome, treating obesity or controlling weight gain, improving reproduction efficiency, treating metabolic disorder, treating hypertension, treating hyperglycemia, treating insulin resistance, treating type 2 diabetes, and/or aiding in cancer and immune therapies. In one embodiment, the glucocorticoid related disorder is stress related or results in stress.

In another embodiment, the synergistic pharmaceutical composition is useful for treating anxiety comprising administering a composition disclosed herein to an animal in need thereof. Also provided is use of a synergistic pharmaceutical composition disclosed herein for treating anxiety in an animal in need thereof. Even further provided is use of a synergistic pharmaceutical composition disclosed herein in the preparation of a medicament for treating anxiety in an animal in need thereof. Also provided is a composition disclosed herein for use in treating anxiety in an animal in need thereof.

In an embodiment, the animal has Post Traumatic Stress Disorder.

In one embodiment, the pharmaceutical compositions are for the treatment of PTSD.

### EXAMPLES

The operation of the disclosure is illustrated by the following representative examples. As is apparent to those skilled in the art, many of the details of the examples may be changed while still practicing the disclosure described herein.

### Materials and Methods

To prepare analogues of betulinic acid, two sources of starting materials were used. Betulin was extracted and purified from white birch bark, and betulinic acid was extracted and purified from sycamore tree bark. For ursolic acid analogues, ursolic acid was extracted and purified from the peels of McIntosh and Fuji apples. For platanic acid analogues, platanic acid was extracted from the bark of American Sycamore (Platanus occidentalis). Materials that were already available in the lab included oleanolic acid, methyl oleanolate, canophyllol analogues and asiatic acid (compounds #1532, 1533, 1542-1545).

All other analogues were prepared using simple, common chemical reactions on the starting materials described above. For analogues that are known in the literature, the structure and purity were confirmed using NMR spectroscopy (¹H and ¹³C) by comparison to literature data. For new analogues, both NMR spectroscopy (¹H and ¹³C) and mass spectrometry were used to confirm the structures and purity. All analogues were obtained at minimum 95% purity, with most being >98%.

Female juvenile rainbow trout (Oncorhynchus mykiss), 75-150 g, were obtained from Linwood Acres Trout Farm (Campellcroft, ON). Fish were transported to the University of Ottawa Aquatic Care Facility and maintained in fiberglass holding tanks (110-115 L) continuously supplied with aerated, dechloraminated City of Ottawa tap water at 13° C. Fish were subjected to a constant 12L:12D photoperiod and fed five times per week with commercial trout pellets (Classic Floating Trout Grower, Martin Mills, Tavistock, ON). All experiments were carried out under protocols approved by the University of Ottawa Protocol Review Committee and adhere to published guidelines of the Canadian Council on Animal Care for the use of animals in teaching and research.

### Example 1

### General Methods for Preparing Esters of Betulinic, Platanic, Oleanolic and Ursolic Acids

### Example 1a

To a solution of the acid in THF at room temperature was added a two times excess of NaH and the solution was stirred for 30 min. To this solution was added 5 mole equivalent of the appropriate alkyl halide and 0.1 mole equivalent of Bu₄NI. The solution was left overnight and then quenched with four volumes of water. Most of the THF was evaporated on a rotary evaporator and the remaining mixture was extracted with 3 times the volume of ethyl acetate. The organic extract was dried over MgSO4, filtered and the solvent was evaporated. The resultant crude product was purified by silica gel chromatography to furnish the pure ester. The products were characterized by 1H and 13 C NMR. New compounds were subjected to HRMS.

### Example 1b

To a stirred solution of the appropriated acid in THF was added 10 equivalents of K₂CO₃ and 5 equivalents of the alkyl halide. The mixture was refluxed for 3 h then cooled to rt. The solvent was evaporated and water was added. The mixture was extracted 3 times with an equal amount ethyl acetate. The combined extracts were dried (Na₂SO₄), filtered and the solvent was evaporated to give the desired ester, which if necessary, was purified and characterized as described above.

### Example 2

### Heptyl Betulinate

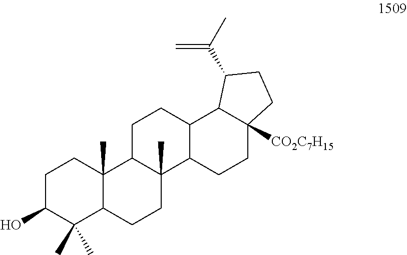

¹H NMR (CDCl₃, 400 MHz): δ (ppm) 4.72 (d, J=2.2 Hz, 1H), 4.59 (dd, J=2.2, 1.3 Hz, 1H), 4.12-4.01 (m, 2H), 3.18 (dd, J=11.2, 5.0 Hz, 1H), 3.04-2.98 (m, 1H), 2.27-2.17 (m, 2H), 1.93-1.84 (m, 2H), 1.68 (s, 3H), 0.96 (s, 6H), 0.91 (s, 3H), 0.81 (s, 3H), 0.75 (s, 3H).

¹³C NMR (CDCl₃, 400 MHz): δ (ppm) 176.3, 150.7, 109.5, 79.0, 64.0, 56.5, 55.3, 50.5, 49.4, 47.0, 42.4, 40.7, 38.8, 38.7, 38.3, 37.2, 37.1, 34.3, 32.2, 31.8, 30.6, 29.6, 28.9, 28.7, 28.0, 27.4, 26.1, 25.5, 22.6, 20.9, 19.4, 18.3, 16.1, 16.0, 15.3, 14.7, 14.0.

### Example 3

### Allyl betulinate

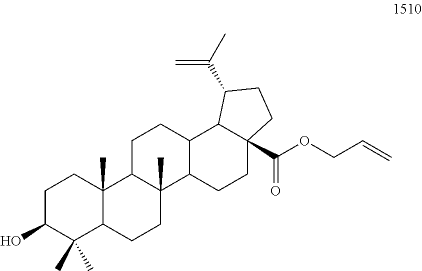

¹H NMR (CDCl₃, 400 MHz): δ (ppm) 5.97-5.87 (m, 1H), 5.33 (dd, J=17.2, 1.5 Hz, 1H), 5.23 (dd, J=10.4, 1.3 Hz, 1H), 4.73 (d, J=2.1 Hz, 1H), 4.63-4.52 (m, 3H), 3.18 (dd, J=11.3, 5.0 Hz, 1H), 3.04-2.98 (m, 1H), 2.28-2.17 (m, 2H), 1.93-1.85 (m, 2H), 1.68 (s, 3H), 0.96 (s, 6H), 0.90 (s, 3H), 0.81 (s, 3H), 0.75 (s, 3H).

¹³C NMR (CDCl₃, 400 MHz): δ (ppm) 175.7, 150.6, 132.5, 118.1, 109.6, 79.0, 64.6, 56.6, 55.3, 50.5, 49.4, 46.9, 42.4, 40.7, 38.8, 38.7, 38.2, 37.2, 37.0, 34.3, 32.1, 30.6, 29.6, 28.0, 27.4, 25.5, 20.9, 19.4, 18.3, 16.1, 15.9, 15.3, 14.7.

### Example 4

### Benzyl betulinate

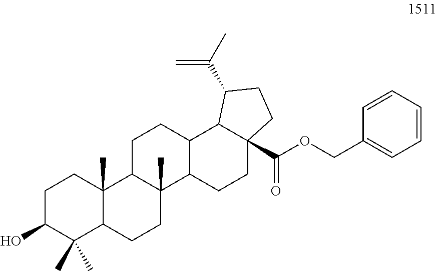

¹H NMR (CDCl₃, 400 MHz): δ (ppm) 7.40-7.30 (m, 5H), 5.15 (d, J=12.3 Hz, 1H), 5.10 (d, J=12.3 Hz, 1H), 4.72 (d, J=2.1 Hz, 1H), 4.59 (dd, J=2.2, 1.3 Hz, 1H), 3.17 (dd, J=11.3, 4.9 Hz, 1H), 3.05-2.98 (m, 1H), 2.30-2.25 (m, 1H), 2.21-2.14 (m, 1H), 1.93-1.82 (m, 2H), 1.67 (s, 3H), 0.95 (s, 3H), 0.94 (s, 3H), 0.79 (s, 3H), 0.76 (s, 3H), 0.75 (s, 3H).

¹³C NMR (CDCl₃, 400 MHz): δ (ppm) 175.8, 150.6, 136.5, 128.5, 128.2, 128.0, 109.6, 79.0, 65.7, 56.5, 55.3, 50.5, 49.4, 46.9, 42.4, 40.6, 38.8, 38.7, 38.2, 37.2, 36.9, 34.3, 32.1, 30.6, 29.6, 28.0, 27.4, 25.5, 20.9, 19.4, 18.3, 16.1, 15.8, 15.3, 14.7.

### Example 5

### Ethyl acetoxy betulinate

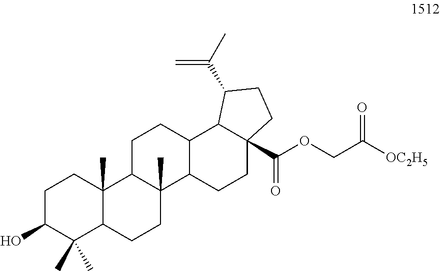

¹H NMR (CDCl₃, 400 MHz): δ (ppm) 4.73 (d, J=2.0 Hz, 1H), 4.59 (dd, J=2.2, 1.4 Hz, 1H), 4.60 (d, J=18.2 Hz, 1H), 4.57 (d, J=18.2 Hz, 1H), 4.26-4.18 (m, 2H), 3.18 (dd, J=11.2, 5.1 Hz, 1H), 3.01-2.95 (m, 1H), 2.33-2.29 (m, 1H), 2.27-2.20 (m, 1H), 2.07-2.02 (m, 1H), 2.00-1.90 (m, 1H), 1.68 (s, 3H), 0.97 (s, 3H), 0.96 (s, 3H), 0.93 (s, 3H), 0.81 (s, 3H), 0.75 (s, 3H).

¹³C NMR (CDCl₃, 400 MHz): δ (ppm) 175.4, 168.1, 150.5, 109.6, 79.0, 61.3, 60.3, 56.5, 55.3, 50.6, 49.4, 46.8, 42.4, 40.7, 38.8, 38.7, 38.1, 37.2, 36.9, 34.3, 31.9, 30.4, 29.5, 28.0, 27.4, 25.5, 20.9, 19.4, 18.3, 16.1, 15.9, 15.3, 14.7, 14.1.

### Example 6

### Methyl canopyllate

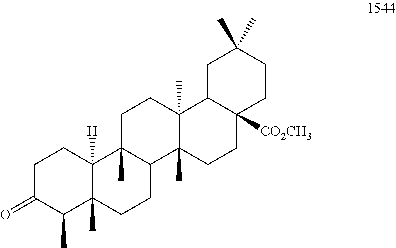

Selected key peaks: ¹H NMR (CDCl₃, 400 MHz): δ (ppm) 3.64 (s, 3H—CO₂CH₃; ¹³C NMR (CDCl₃, 400 MHz): δ (ppm) 213.3 (C═O, ketone), 179.4 (C═O, methyl ester, 58.2 (OCH₃).

HRMS: Calculated for C₃₁H₅₀O₃, 470.3760. Found 482.3755.

### Example 7

### Ethyl acetoxy canophyllate

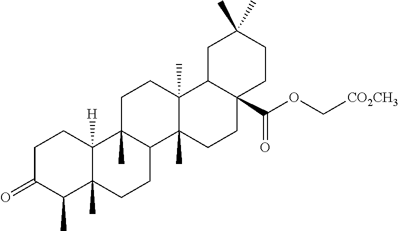

Selected key peaks: ¹H NMR (CDCl₃, 400 MHz): δ (ppm) 4.58 (AB quartet, 2H); 3.73 (9s, 3H).

¹³C NMR (CDCl₃, 400 MHz): δ (ppm) 213.1 (C═O, ketone), 178.1 (C═O, ester) 168.5 (C═O, ester), 58.2 (OCH₃).

HRMS: Calculated for C₃₃H₅₂O₅, 528.3815. Found 528.3806.

### Example 8

### Preparation of Amides of the Betulinic Acid

Generally, 3-acetoxy acid was converted into its acid chloride using oxalyl chloride in dichloromethane containing a catalytic amount of DMF. The mixture was stirred at room temperature for six hours. The solvent was evaporated and the residue was re-dissolved in dichloromethane and added to a solution of 1.1 equivalents of the appropriate amine containing also 1.1 equivalents of triethylamine. The mixture was stirred for 1 h and then washed successively with water, 1% HCl solution and again water. The organic extracts were dried over MgSO₄, filtered and the solvent was evaporated. The 3-acetoxy group was removed by stirring with K₂CO₃ in methanol overnight at 35° C. Usual further workup afforded the crude amides which were purified by column chromatography.

### Example 9

### Betulinic acid glycine amide

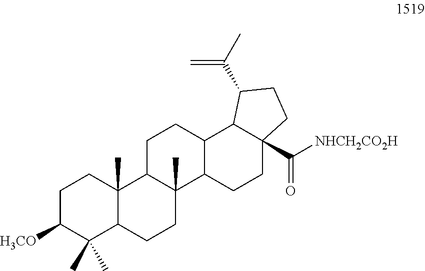

¹H NMR (CDCl₃, 400 MHz: δ (ppm) 6.11 (br s, 1H), 4.73 (s, 1H), 4.60 (s, 1H), 4.07 (dd, J=18.1, 5.2 Hz, 1H), 4.04 (dd, J=18.1, 5.2 Hz, 1H), 3.19 (dd, J=11.2, 5.0 Hz, 1H), 3.12-3.06 (m, 1H), 2.43-2.36 (m, 1H), 2.04-1.90 (m, 2H), 1.68 (s, 3H), 0.97 (s, 3H), 0.96 (s, 3H), 0.92 (s, 3H), 0.81 (s, 3H), 0.75 (s, 3H), 0.68 (br d, J=8.9 Hz, 1H).

¹³C NMR (CDCl₃, 400 MHz: δ (ppm) 177.4, 173.5, 150.7, 109.5, 79.1, 55.8, 55.4, 50.6, 50.0, 46.7, 42.5, 41.4, 40.7, 38.8, 38.7, 38.3, 37.8, 37.2, 34.4, 33.7, 30.7, 29.4, 28.0, 27.4, 25.6, 20.9, 19.5, 18.3, 16.1, 16.0, 15.4, 14.6.

### Example 10

### Betulinic acid isopropyl amide

¹H NMR (CDCl₃, 400 MHz, δ (ppm) 5.61 (t, 1H, NH) 4.71 (d, J=2 Hz, 1H), 4.56 (d, J=2 Hz 1H), 3.12 (m, 3H), 2.98 (m, 1H), 2.46 (dt J=12.3, 3.2 Hz, 1H), 1.65 (s, 3H), 0.91 (s, 3H), 0.96 (s, 3H), 0.89 (s, 3H), 0.88 (s, 3H), 0.79 (s, 3H), 0.73 (s. 3H).

¹³C NMR (CDCl₃, 400 MHz): δ (ppm) 175.9, 150.9, 109.2, 78.9, 55.6, 55.4, 50.8, 50.2, 46.7, 46.6, 42.5, 40.7, 38.8, 38.7, 38.5, 37.7, 37.2, 34.4, 33.9, 30.9, 29.4, 28.7, 27.9, 27.4, 25.6, 20.9, 20.2, 19.4, 18.3, 16.1, 16.0, 15.3, 14.6

HRMS: Calc'd for C34H57NO2: 511.4392. Found: 511.4405.

### Example 11

### 3-Methoxy betulinic acid

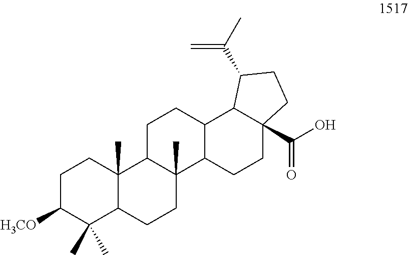

60% NaH in mineral oil (104 mg, 2.60 mmol) under N₂ was rinsed with dry THF (5 mL) and then suspended in dry THF (5 mL). A solution of betulinic acid (200 mg, 0.44 mmol) in dry THF (9 mL) was added and stirred 30 min. CH₃I was added (0.33 mL, 5.30 mmol) and stirred overnight. Sat'd NH₄Cl solution (10 mL) and water (10 mL) was added and the solvent evaporated. The residue was extracted with EtOAc (3×15 mL), dried (Na₂SO₄), filtered and solvent evaporated to give a crude white solid (183 mg). Purification of the solid by silica gel chromatography using a hexanes/EtOAc gradient gave the desired compound as a white powder (39 mg, 19%).

¹H NMR (CDCl₃, 400 MHz, centered on TMS): δ (ppm) 11.36 (br, 1H), 4.74 (d, J=1.4 Hz, 1H), 4.61 (s, 1H), 3.35 (s, 3H), 3.04-2.98 (m, 1H), 2.64 (dd, J=11.7, 4.3 Hz, 1H), 2.30-2.25 (m, 1H), 2.22-2.15 (m, 1H), 2.05-1.94 (m, 2H), 1.69 (s, 3H), 0.97 (s, 3H), 0.95 (s, 3H), 0.93 (s, 3H), 0.83 (s, 3H), 0.73 (s, 3H).

¹³C NMR (CDCl₃, 400 MHz): δ (ppm) 182.5, 150.4, 109.7, 88.7, 57.5, 56.4, 55.9, 50.5, 49.3, 46.9, 42.4, 40.7, 38.8, 38.6, 38.4, 37.2, 37.0, 34.3, 32.2, 30.6, 29.7, 28.0, 25.5, 22.2, 20.8, 19.4, 18.1, 16.1, 16.0, 14.7.

HRMS: Calculated for C₃₁H₅₀O₃, 470.37600. Found 470.38018

### Example 12

### Methyl 3-methoxybetulinate

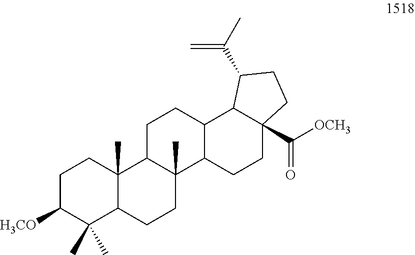

The procedure described in Example 11 also gave 70 mg of this compound as a white powder after silica gel chromatography (33%).

¹H NMR (CDCl₃, 400 MHz, centered on TMS): δ (ppm) 4.74 (s, 1H), 4.60 (s, 1H), 3.67 (s, 3H), 3.35 (s, 3H), 3.03-2.98 (m, 1H), 2.63 (dd, J=11.7, 4.1 Hz, 1H), 2.28-2.16 (m, 2H), 1.94-1.84 (m, 2H), 1.69 (s, 3H), 0.96 (s, 3H), 0.94 (s, 3H), 0.91 (s, 3H), 0.82 (s, 3H), 0.74 (s, 3H).

¹³C NMR (CDCl₃, 400 MHz): δ (ppm) 176.6, 150.6, 109.5, 88.6, 57.5, 56.6, 55.9, 51.3, 50.5, 49.5, 47.0, 42.4, 40.7, 38.8, 38.6, 38.3, 37.2, 37.0, 34.3, 32.2, 30.6, 29.7, 28.0, 25.5, 22.2, 20.9, 19.4, 18.2, 16.11, 16.07, 16.0, 14.7.

HRMS: Calculated for C₃₂H₅₂O₃, 484.39165. Found 484.39340.

### Example 13

### Methyl 3-acetoxybetulinate

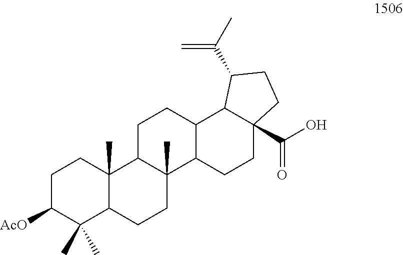

A solution of methyl betulinate (2.00 g, 4.25 mmol), DMAP (23 mg), Et₃N (2.07 mL, 14.9 mmol), and Ac₂O (1.20 mL, 12.8 mmol) in DCM (80 mL) was stirred overnight at rt. In the morning, 5% HCl was added (40 mL) and stirred 30 min. Layers were separated and aqueous phase extracted with DCM (80 mL). The combined DCM extracts were washed with H₂O (50 mL) and brine (50 mL), dried (MgSO₄), filtered and the solvent evaporated to give a yellow solid (2.17 g). The solid was purified by silica gel chromatography using a hexanes/EtOAc gradient to give 2.01 g (92%) of the title compound as a white powder.

¹H NMR (CDCl₃, 400 MHz): δ (ppm) 4.73 (d, J=2.2 Hz, 1H), 4.60 (dd, J=2.3, 1.4 Hz, 1H), 4.48-4.44 (m, 1H), 3.66 (s, 3H), 3.02-2.96 (m, 1H), 2.04 (s, 3H), 1.68 (d, J=0.5 Hz, 3H), 0.95 (s, 3H), 0.91 (s, 3H), 0.84 (s, 6H), 0.83 (s, 3H).

¹³C NMR (CDCl₃, 400 MHz): δ (ppm) 176.7, 171.0, 150.6, 109.6, 80.9, 56.5, 55.4, 51.2, 50.4, 49.4, 47.0, 42.4, 40.7, 38.4, 38.2, 37.8, 37.1, 36.9, 34.2, 32.1, 30.6, 29.6, 27.9, 25.4, 23.7, 21.3, 20.9, 19.3, 18.2, 16.5, 16.2, 15.9, 14.7.

### Example 14

### Methyl 3-(4-bromobenzoyl)-betulinate

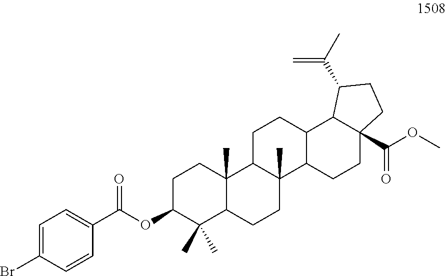

To a stirred solution of methyl betulinate (250 mg, 0.53 mmol) in DCM (5 mL) was added DMAP (10 mg), Et₃N (0.59 mL, 4.23 mmol) and 4-bromobenzoyl chloride (0.69 g, 3.14 mmol). The solution was stirred overnight, diluted with DCM (15 mL), washed with 5% HCl (10 mL) and H₂O (10 mL), dried (MgSO₄), filtered and the solvent evaporated. Purification of the crude material by silica gel chromatography using a hexanes/EtOAc gradient gave 304 mg (88%) of the title compound as a white powder.

¹H NMR (CDCl₃, 300 MHz): δ (ppm) 7.91-7.87 (m, 2H), 7.59-7.55 (m, 2H), 4.74-4.67 (m, 2H), 4.61-4.60 (m, 1H), 3.67 (s, 3H), 3.05-2.96 (m, 1H), 2.26-2.16 (m, 2H), 1.69 (s, 3H), 0.98 (s, 3H), 0.97 (s, 3H), 0.93 (s, 3H), 0.90 (s, 3H), 0.89 (s, 3H)

¹³C NMR (CDCl₃, 400 MHz): δ (ppm) 176.7, 165.6, 150.5, 131.6, 131.0, 129.9, 127.8, 109.6, 82.0, 56.5, 55.5, 51.3, 50.5, 49.4, 47.0, 42.4, 40.7, 38.4, 38.24, 38.18, 37.1, 37.0, 34.2, 32.2, 30.6, 29.7, 28.1, 25.5, 23.7, 20.9, 19.3, 18.2, 16.8, 16.2, 16.0, 14.7

HRMS: Calculated for C₃₈H₅₃BrO₄, 652.31272. Found 652.31387.

### Example 15

### Methyl 3β-amido-betulinate and methyl 3-α-amido-betulinate

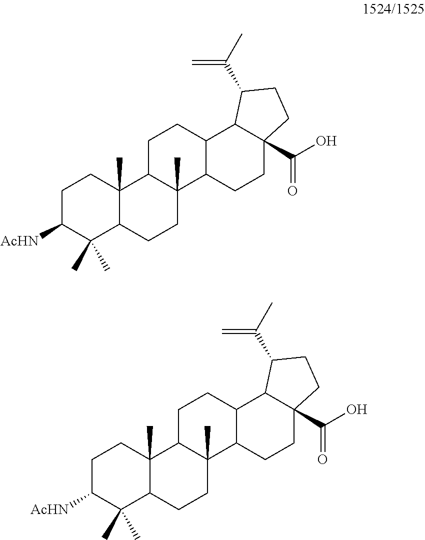

A solution of methyl betulonate (100 mg, 0.214 mmol), NH₄OAc (165 mg, 2.14 mmol), NaBH₃CN (9.4 mg, 0.15 mmol) and activated 3 Å molecular sieves in MeOH (10 mL) was stirred for two days. Additional NaBH₃CN (10 mg, 0.16 mmol) was added and stirring continued for three more days. The solution was filtered and acidified to pH ˜1 with 30% HCl. The solvent was evaporated and water (10 mL) and Et₂O (10 mL) were added. A white precipitate formed which was filtered off (49 mg). The layers were separated and aqueous layer extracted with EtOAc (2×10 mL). The organic layers were combined, dried (MgSO₄), filtered, and solvent evaporated to give pale orange solid (75 mg).

The orange solid and white precipitate were dissolved in DCM (6 mL) with Ac₂O (0.08 mL, 0.85 mmol), Et₃N (0.13 mL, 0.93 mmol) and DMAP (3 mg). After stirring overnight at rt, DCM (10 mL) and 5% HCl (10 mL) was added and stirred 1 hr. Layers were separated and the organic layer washed with water (10 mL) and brine (10 mL), dried (MgSO₄), filtered and solvents evaporated to give a white solid (142 mg). Separation of the solid by silica gel chromatography with a hexanes/EtOAc gradients gave 54 mg (48%) of the acetylated and 18 mg, (16%) of the α-isomer as white solids.

β-Isomer: ¹H NMR (CDCl₃, 400 MHz): δ (ppm) 5.27 (d, J=10.0 Hz, 1H), 4.73 (d, J=2.2 Hz, 1H), 4.59 (dd, J=2.3, 1.4 Hz, 1H), 3.64 (ddd, J=12.2, 10.0, 4.4 Hz, 1H), 3.66 (s, 3H), 3.02-2.95 (m, 1H), 1.98 (s, 3H), 1.68 (s, 3H), 0.95 (s, 3H), 0.90 (s, 3H), 0.86 (s, 3H), 0.80 (s, 3H), 0.73 (s, 3H). ¹³C NMR (CDCl₃, 400 MHz): δ (ppm) 176.7, 169.5, 150.6, 109.6, 56.6, 56.5, 56.0, 51.2, 50.4, 49.4, 47.0, 42.4, 40.6, 39.1, 38.2, 37.7, 37.0, 36.9, 34.2, 32.1, 30.6, 29.6, 28.3, 25.6, 25.5, 23.7, 20.8, 19.3, 18.5, 16.4, 16.0, 15.9, 14.6. HRMS: Calculated for C₃₃H₅₃NO₃, 511.40254. Found 511.40075

α-Isomer: ¹H NMR (CDCl₃, 300 MHz): δ (ppm) 5.71 (d, J=9.8 Hz, 1H), 4.73 (d, J=2.0 Hz, 1H), 4.60 (dd, J=2.1, 1.4 Hz), 3.81 (dt, J=9.8, 3.2 Hz, 1H), 3.66 (s, 3H), 3.04-2.95 (m, 1H), 2.02 (s, 3H), 1.68 (s, 3H), 1.01 (s, 3H), 0.923 (s, 3H), 0.919 (s, 3H), 0.85 (s, 3H), 0.83 (s, 3H). ¹³C NMR (CDCl₃, 400 MHz): 6 (ppm) 176.6, 169.3, 150.5, 109.6, 56.5, 53.9, 52.0, 51.3, 50.8, 49.4, 46.9, 42.5, 40.8, 38.1, 37.4, 36.9, 36.4, 34.5, 34.1, 32.1, 30.6, 29.6, 28.3, 25.5, 23.8, 23.3, 22.9, 20.7, 19.4, 18.1, 16.02, 15.96, 14.9. HRMS: Calculated for C₃₃H₅₃NO₃, 511.40254. Found 511.40345.

### Example 16

### Methyl 3β-(N-benzyl)acetamido betulinate

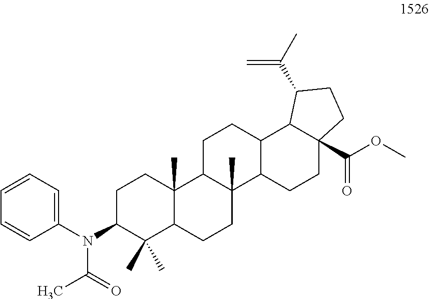

A solution of methyl betulonate (100 mg, 0.214 mmol), benzylamine (0.14 mL, 1.28 mmol), glacial acetic acid (0.04 mL, 0.75 mmol), NaBH₃CN (9.4 mg, 0.15 mmol) and activated 3 Å molecular sieves in MeOH (10 mL) was stirred for two days. Additional NaBH₃CN (10 mg, 0.16 mmol) was added and stirring continued for three days. The solution was filtered and acidified to pH ˜1 with 30% HCl. The solvent was evaporated, water added (10 mL) and the mixture extracted with Et₂O (3×8 mL). The aqueous layer was made basic (pH 10) by adding K₂CO₃ and extracted with EtOAc (2×10 mL). All organic layers were combined, dried (MgSO₄), filtered and solvent evaporated. A white precipitate formed in the aqueous layer, which was filtered off and combined with the organic extracts to give a white solid (123 mg).

The white solid was dissolved in DCM (6 mL) with Et₃N (0.30 mL, 2.15 mmol), Ac₂O (0.16 mL, 1.70 mmol), and DMAP (3 mg). After stirring overnight at rt, DCM (10 mL) and 5% HCl (10 mL) was added and stirred 1 hr. Layers were separated and the organic layer was washed with H₂O (10 mL) and brine (10 mL), dried (MgSO₄), filtered and solvent evaporated to give a pale oil (142 mg). Separation of the oil by silica gel chromatography using a hexanes/EtOAc gradient gave acetylated amine, β-isomer as a white powder (43 mg, 33%).

¹H NMR (CDCl₃, 300 MHz): δ (ppm) 7.36-7.13 (m, 5H), 4.88 (d, J=16.2 Hz, CH₂Ph minor rotamer, 1H), 4.72 (d, J=1.7 Hz, H-29, 1H), 4.64 (dd, J=12.5, 3.1 Hz, H-3 major rotamer, 1H), 4.59 (br s, H-29 [1H] and CH₂Ph major rotamer [2H]), 4.30 (d, J=15.9 Hz, CH₂Ph minor rotamer, 1H), 3.66 (s, CO₂CH₃ minor rotamer, 3H), 3.65 (s, CO₂CH₃ major rotamer, 3H), 3.52 (dd, J=12.6, 3.5 Hz, H-3 minor rotamer, 1H), 3.03-2.94 (m, 1H), 2.27 (s, NCOCH₃ minor rotamer, 3H), 1.97 (s, NCOCH₃ major rotamer, 3H), 1.68 (s, 3H), 0.96 (s, 3H), 0.89 (s, 6H), 0.86 (s, 3H), 0.79 (s, 3H). HRMS: Calculated for C₄₀H₅₉NO₃, 601.4495. Found 601.4473.

### Example 17

### Vinylogous ethyl betulonate

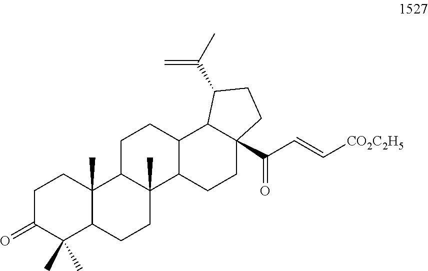

To a stirred suspension of 60% NaH in mineral oil (246 mg, 6.15 mmol) in dry toluene (8 mL) was slowly added triethyl phosphonoacetate (1.29 mL, 6.50 mmol). After 20 min, a solution of betulonaldehyde (900 mg, 2.05 mmol) in dry toluene (7 mL) was added and heated to reflux for 4 hrs. The solution was cooled to 0° C. and quenched with saturated NH₄Cl solution (15 mL). It was diluted with hexanes (100 mL) and water (30 mL), shaken and layers separated. The organic extract was washed with water (50 mL) and brine (40 mL), dried (MgSO₄), filtered and solvent evaporated to give a colourless oil (1.29 g). Purification of the oil by silica gel chromatography using a hexanes/EtOAc gradient gave 888 mg (85%) of the desired Wittig product as white foam.

¹H NMR (CDCl₃, 400 MHz): δ (ppm) 7.25 (d, J=16.2 Hz, 1H), 5.89 (d, J=16.2 Hz, 1H), 4.72 (d, J=1.2 Hz, 1H), 4.61 (s, 1H), 4.21 (q, J=7.1 Hz, 2H), 2.55-2.45 (m, 2H), 2.38 (ddd, J=15.6, 7.5, 4.4 Hz, 1H), 1.69 (s, 3H), 1.31 (t, J=7.2, 3H), 1.06 (s, 3H), 1.01 (s, 3H), 0.99 (s, 3H), 0.98 (s, 3H), 0.92 (s, 3H).

¹³C NMR (CDCl₃, 400 MHz): δ (ppm) 218.1, 167.1, 153.5, 149.7, 120.4, 110.1, 60.3, 54.9, 50.0, 49.7, 49.6, 47.6, 47.3, 42.9, 40.7, 39.6, 38.9, 38.7, 36.9, 34.1, 33.6, 33.3, 29.7, 27.7, 26.6, 25.2, 21.3, 21.0, 19.6, 19.3, 15.9, 15.8, 14.6, 14.3.

HRMS: Calculated for C₃₄H₅₂O₃, 508.39165. Found 508.38972.

### Example 18

### Vinylogous betulonic Acid

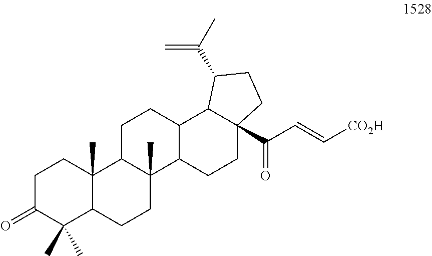

To a stirred solution of ester from Example 15 (71 mg, 0.139 mmol) in MeOH (6 mL) was added water (0.2 mL) and KOH pellets (313 mg, 5.58 mmol). It was heated to 45° C. for 4 hrs until TLC showed complete disappearance of starting material (9:1 hexanes:EtOAc). The solution was cooled to rt, acidified with 30% HCl and solvent evaporated. It was diluted with water (5 mL) and extracted with EtOAc (3×15 mL). The organic extract was washed with brine (2×10 mL), dried (MgSO₄), filtered and solvent evaporated to give white foam (67 mg). Purification of the foam by silica gel chromatography using 4:1 hexanes:EtOAc gave 56 mg (84%) of the desired acid as white foam.

¹H NMR (CDCl₃, 400 MHz): δ (ppm) 7.38 (d, J=16.2 Hz, 1H), 5.93 (d, J=16.1 Hz, 1H), 4.73 (d, J=1.7 Hz, 1H), 4.62 (d, J=1.4 Hz, 1H), 2.56-2.45 (m, 2H), 2.39 (ddd, J=15.6, 7.5, 4.3 Hz, 1H), 1.70 (s, 3H), 1.07 (s, 3H), 1.02 (s, 3H), 0.99 (s, 6H), 0.92 (s, 3H).

¹³C NMR (CDCl₃, 400 MHz): δ (ppm) 218.2, 171.9, 156.6, 149.6, 119.8, 110.2, 54.9, 50.3, 49.7, 49.6, 47.7, 47.3, 42.9, 40.7, 39.6, 39.1, 38.8, 36.9, 34.1, 33.6, 33.1, 29.7, 27.8, 26.6, 25.2, 21.2, 21.0, 19.6, 19.3, 15.9, 15.8, 14.6.

HRMS: Calculated for C₃₂H₄₈O₃, 480.36035. Found 480.35958.

### Example 19

### Vinylogous ethyl betulinate and vinylogous ethyl epi-betulinate

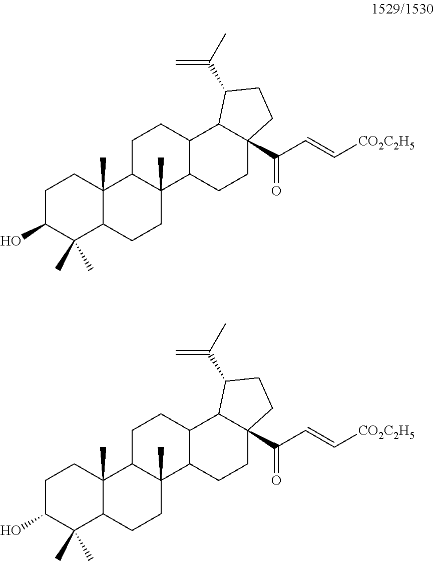

To a stirred solution of ethyl ester from Example 16, (200 mg, 0.393 mmol) in MeOH (10 mL) at 0° C. was added NaBH₄ (15 mg). After 30 min, it was acidified with 10% HCl (10 mL), the solvent was evaporated and the residue extracted with EtOAc (2×20 mL). The extract was washed with water (15 mL) and brine (10 mL), dried (MgSO₄), filtered and solvent evaporated to give a white solid (185 mg). Purification of the solid by silica gel chromatography using a hexanes/EtOAc gradients gave 126 mg (63%) of the vinylogous ethyl betulinate and as a white powder and 7 mg (3%) of the 3-epi-isomer as a white foam

Vinylogous ethyl betulinate: ¹H NMR (CDCl₃, 400 MHz): δ (ppm) 7.25 (d, J=16.2 Hz, 1H), 5.89 (d, J=16.2 Hz, 1H), 4.60 (dd, J=2.0, 1.6 Hz, 1H), 4.21 (qd, J=7.2, 1.1 Hz, 2H), 3.18 (dd, J=11.3, 5.0 Hz, 1H), 2.54-2.47 (m, 1H), 1.69 (d, J=0.5 Hz, 3H), 1.31 (t, J=7.1, 3H), 0.97 (s, 3H), 0.96 (s, 3H), 0.95 (s, 3H), 0.81 (s, 3H), 0.75 (s, 3H). ¹³C NMR (CDCl₃, 400 MHz): δ (ppm) 167.1, 153.6, 149.8, 120.3, 110.0, 79.0, 60.2, 55.3, 50.4, 50.0, 49.7, 47.7, 42.8, 40.8, 38.84, 38.75, 38.7, 37.1, 34.3, 33.4, 29.7, 28.0, 27.8, 27.4, 25.2, 20.7, 19.2, 18.3, 16.1, 16.0, 15.3, 14.7, 14.3. HRMS: Calculated for C₃₄H₅₄O₃, 510.4073. Found 510.4096

Vinylogous ethyl 3-epi-betulinate: ¹H NMR (CDCl₃, 400 MHz): δ (ppm) 7.26 (d, J=16.1 Hz, 1H), 5.89 (d, J=16.2 Hz, 1H), 4.72 (d, J=1.7 Hz, 1H), 4.60 (s, 1H), 4.21 (q, J=7.0 Hz, 2H), 3.38 (t, J=2.4 Hz, 1H), 2.54-2.47 (m, 1H), 1.68 (s, 3H), 1.31 (t, J=7.1, 3H), 0.99 (s, 3H), 0.96 (s, 3H), 0.93 (s, 3H), 0.82 (s, 3H), 0.82 (s, 3H). ¹³C NMR (CDCl₃, 400 MHz): δ (ppm) 167.1, 153.7, 149.9, 120.3, 110.0, 76.2, 60.2, 50.1, 50.0, 49.7, 49.0, 47.7, 42.9, 41.0, 38.71, 38.66, 37.5, 37.3, 34.1, 33.4, 33.2, 29.7, 28.2, 27.7, 25.4, 25.2, 22.1, 20.6, 19.2, 18.2, 16.0, 15.9, 14.8, 14.3. HRMS: Calculated for C₃₄H₅₄O₃, 510.40730. Found 510.4074

### Example 20

### Vinylogous betulinic acid

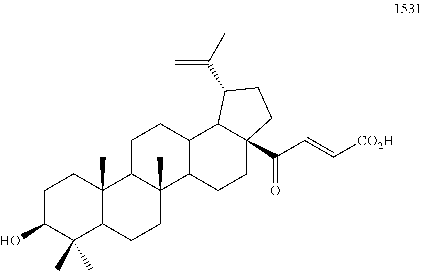

To a stirred solution of vinylogous ethyl betulinate obtained in Example #19 (40 mg, 0.083 mmol) in MeOH (4 mL) was added water (0.1 mL) and KOH pellets (210 mg). It was heated to 45° C. for 5 hrs, cooled to rt, and solvent evaporated. The residue was acidified with 30% HCl, diluted with water (5 mL) and extracted with EtOAc (2×20 mL). The extract was washed with brine (10 mL), dried (MgSO₄), filtered and solvent evaporated. Purification of the residue by silica gel chromatography using a hexanes/EtOAc gradient gave vinylogous betulinic acid as a white powder (18 mg, 45%).

¹H NMR (CDCl₃, 400 MHz): δ (ppm) 7.37 (d, J=16.2 Hz, 1H), 5.92 (d, J=16.1 Hz, 1H), 4.73 (d, J=1.7 Hz, 1H), 4.61 (dd, J=1.7, 1.4 Hz, 1H), 3.19 (dd, J=11.2, 5.0 Hz, 1H), 2.55-2.48 (m, 1H), 1.70 (s, 3H), 0.98 (s, 3H), 0.96 (s, 3H), 0.95 (s, 3H), 0.81 (s, 3H), 0.76 (s, 3H).

¹³C NMR (CDCl₃, 400 MHz): δ (ppm) 171.6, 156.6, 149.7, 1197, 110.1, 79.0, 55.3, 50.4, 50.3, 49.7, 47.7, 42.8, 40.8, 39.0, 38.84, 38.76, 38.68, 37.1, 34.3, 33.2, 29.7, 28.0, 27.8, 27.3, 25.2, 20.7, 19.3, 18.3, 16.1, 16.0, 15.4, 14.7.

HRMS: Calculated for C₃₂H₅₀C₃, 482.37600. Found 482.37416

### Example 21

### Methyl 2β-allyl-betulonate

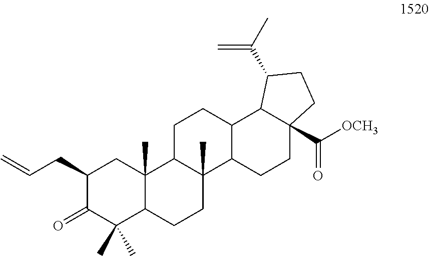

A solution of fresh LDA was prepared by dissolving dry i-Pr₂NH (25 μL, 0.18 mmol) in dry THF (4 mL) under N₂, cooling to −78° C., adding 1.6M n-BuLi in hexanes (1094, 0.17 mmol) and stirring for 15 min. To the LDA was added a solution of methyl betulonate (80 mg, 0.17 mmol) in dry THF (1 mL) under N₂. After 30 min, freshly distilled allyl bromide (16 μL, 0.19 mmol) was added and stirred 2 hrs before warming to 0° C. After 2 hrs, the solution was warmed to rt overnight. It was then quenched with sat′d NH₄Cl (10 mL) and solvent evaporated. The residue was dissolved in EtOAc (40 mL), washed with brine (2×10 mL), dried (Na₂SO₄), filtered and solvent evaporated to give a colourless oil (85 mg). Separation of the oil by silica gel chromatography using a hexanes/EtOAc gradient gave the 2β-allyl derivative as white foam (13 mg, 15%) along with recovered starting material (43 mg, 54%).

¹H NMR (CDCl₃, 400 MHz, centered on TMS): δ (ppm) 5.80-5.69 (m, 1H), 5.03-4.96 (m, 2H), 4.74 (d, J=2.0 Hz, 1H), 4.60 (dd, J=2.0, 1.4 Hz, 1H), 3.68 (s, 3H), 3.03-2.97 (m, 1H), 2.73-2.65 (m, 1H), 2.57-2.51 (m, 1H), 2.26-2.19 (m, 2H), 2.07 (dd, J=13.2, 5.7 Hz, 1H), 1.68 (s, 3H), 1.08 (s, 3H), 1.05 (s, 3H), 1.04 (s, 3H), 0.97 (s, 3H), 0.95 (s, 3H).

¹³C NMR (CDCl₃, 400 MHz): δ (ppm) 217.2, 176.6, 150.5, 136.9, 116.2, 109.7, 57.3, 56.5, 51.3, 50.1, 49.4, 48.3, 47.0, 46.9, 42.5, 41.5, 40.8, 38.2, 37.4, 37.0, 34.5, 34.1, 32.1, 30.6, 29.6, 25.4, 25.3, 21.7, 21.1, 19.3, 19.3, 16.10, 16.08, 14.6.

### Example 22

### Methyl 2α-hydroxy betulonate

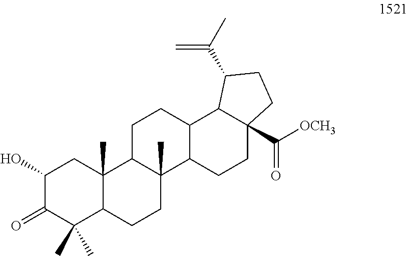

To a stirred solution of methyl betulonate (200 mg, 0.427 mmol) in dry DCM (8.7 mL) under N₂ at −78° C. was added dry Et₃N (0.60 mL, 4.27 mmol) followed by TMSOTf (0.39 mL, 2.14 mmol). After 1 hr the reaction was quenched with saturated NaHCO₃ solution (4 mL) and warmed to rt. Layers were separated and aqueous layer extracted with hexanes (3×5 mL). The organic extracts were combined, washed with brine (5 mL), dried (MgSO₄), filtered and evaporated to give crude silyl enol ether (218 mg, 0.40 mmol; 95%) as a single spot on TLC (9:1 hexanes:EtOAc, Rf=0.79).

Crude silyl enol ether was dissolved in DCM (8 mL) and cooled to 0° C. and a solution of ˜77% mCPBA (100 mg, 0.44 mmol) in DCM (10 mL) was cooled to 0° C. and added. After 1 hr 40 min TLC showed complete disappearance of starting material. The reaction was quenched with saturated NaHCO₃ (7 mL), stirred 30 min and layers separated. The organic layer was evaporated to give the expected epoxide which was dissolved in MeOH (10 mL) with 5% HCl (1 mL). After 30 min stirring, TLC showed hydrolysis was complete. Solvent was evaporated and the residue dissolved in EtOAc (20 mL). It was washed with water (8 mL) and brine (8 mL), dried (MgSO₄), filtered and solvent evaporated to give a colourless oil (203 mg). Separation of the oil by silica gel chromatography using 15% EtOAc in hexanes gave the desired 2-a-hydroxy compound as white foam (128 mg, 62%).

¹H NMR (CDCl₃, 400 MHz): δ (ppm) 4.72 (br s, 1H), 4.59 (br s, 1H), 4.52 (dd, J=12.5, 6.7 Hz, 1H), 3.66 (s, 1H), 3.00-2.94 (m, 1H), 2.45 (dd, J=12.5, 6.7 Hz, 1H), 2.24-2.17 (m, 2H), 1.66 (s, 3H), 1.15 (s, 3H), 1.12 (s, 3H), 1.07 (s, 3H), 0.95 (s, 3H), 0.92 (s, 3H).

¹³C NMR (CDCl₃, 400 MHz): δ (ppm) 216.7, 176.6, 150.3, 109.7, 69.3, 57.9, 56.4, 51.3, 50.2, 49.9, 49.4, 47.7, 46.9, 42.5, 40.8, 38.1, 38.0, 36.9, 34.0, 32.1, 30.5, 29.6, 25.2, 24.5, 21.3, 21.2, 19.3, 19.1, 16.6, 16.1, 14.6.

HRMS: Calculated for C₃₁H₄₈O₄, 484.3553. Found 484.3545.

### Example 23

### Methyl 2α-hydroxy betulinate and methyl 2β-hydroxy-3-epibetulinate

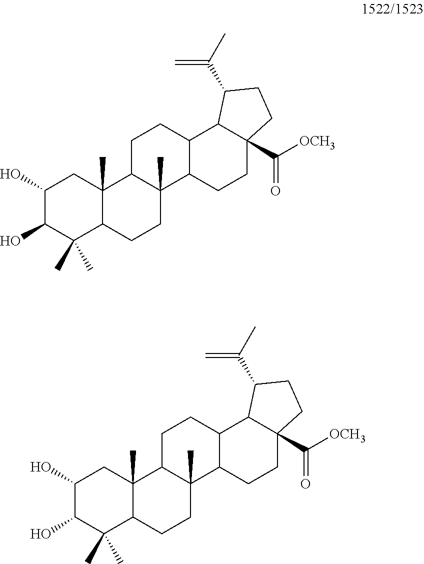

To a stirred solution of the ketone obtained in Example 20 (69 mg, 0.14 mmol) in MeOH (6 mL) at 0° C. was added NaBH₄ (22 mg, 0.58 mmol). After 30 min it was acidified with 10% HCl (5 mL) and the solvent evaporated. The residue was extracted with EtOAc (3×5 mL), washed with brine (6 mL), dried (MgSO₄), filtered and solvent evaporated to give a white powder (64 mg). Separation of the powder by silica gel chromatography using a hexanes/EtOAc gradient gave 41 mg (60%) of 2,2-hydroxy methyl betulinate as a white foam and 10 mg (15%) of the 3-epi isomers also as a white foam.

Methyl 2α-hydroxy betulinate: ¹H NMR (CDCl₃, 400 MHz): δ (ppm) 4.73 (s, 1H), 4.60 (s, 1H), 3.70-3.64 (m, 1H) 3.66 (s, 3H), 3.02-2.96 (m, 1H), 2.96 (d, J=9.5 Hz, 1H), 2.24-2.15 (m, 2H), 1.68 (s, 3H), 1.57 (t, J=11.3 Hz, 1H), 1.00 (s, 3H), 0.96 (s, 3H), 0.91 (s, 3H), 0.89 (s, 3H), 0.79 (s, 3H). ¹³C NMR (CDCl₃, 400 MHz): δ (ppm) 176.6, 150.4, 109.6, 83.9, 69.2, 56.5, 55.5, 51.3, 50.5, 49.4, 47.0, 46.8, 42.4, 40.7, 39.2, 38.6, 38.2, 36.9, 34.2, 32.2, 30.6, 29.6, 28.5, 25.4, 21.0, 19.4, 18.3, 17.4, 16.5, 16.0, 14.7.

Methyl 2β-hydroxy-3-epibetulinate: ¹H NMR (CDCl₃, 400 MHz): δ (ppm) 4.73 (d, J=2.1 Hz, 1H), 4.60 (dd, J=2.2, 1.3 Hz, 1H), 3.98 (br d, J=10.0 Hz, 1H), 3.41 (d, J=2.7 Hz, 1H), 3.02-2.96 (m, 1H), 2.24-2.15 (m, 2H), 1.68 (s, 3H), 1.57 (t, J=11.4 Hz, 1H), 0.99 (s, 3H), 0.97 (s, 3H), 0.90 (s, 3H), 0.86 (s, 3H), 0.83 (s, 3H). ¹³C NMR (CDCl₃, 400 MHz): δ (ppm) 176.7, 150.5, 109.6, 78.9, 66.7, 56.6, 51.2, 50.2, 49.4, 48.2, 47.0, 42.5, 42.2, 40.9, 38.6, 38.3, 38.2, 37.0, 34.1, 32.2, 30.6, 29.6, 28.5, 25.4, 21.6, 20.8, 19.3, 18.0, 17.1, 16.0, 14.8.

### Example 24

### Methyl 29-hydroxy-dihydrobetulinate

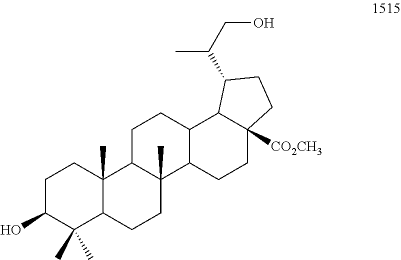

BH3.S(CH₃)₂ 64.6 mg was added to a solution of 200 mg of methyl betulinate dissolved in 15 ml of THF under nitrogen at room temperature. The solution was allowed to stand for 7 h, then reacted with 0.43 mL of 3M NaOH followed by 1 mL of 30% H2O2. The mixture was warmed to 50° C. and kept at that temperature overnight. Typical workup gave a crude product which was purified by silica gel chromatography. The yield of the title compound, a white solid was 140 m (67%).

¹H NMR (CDCl₃, 400 MHz): δ (ppm) 3.78 (dd, J=10.4, 4.5 Hz, 1H), 3.64 (s, 3H), 3.41 (dd, J=10.3, 8.1 Hz, 1H), 3.19 (dd, J=11.3, 5.0 Hz, 1H), 0.96 (s, 3H), 0.96 (d, J=6.8 Hz, 3H), 0.94 (s, 3H), 0.90 (s, 3H), 0.82 (s, 3H), 0.75 (s, 3H).

¹³C NMR (CDCl₃, 400 MHz): d (ppm) 176.6, 78.9, 64.3, 56.8, 55.3, 51.2, 50.2, 48.8, 43.2, 42.5, 40.6, 38.8, 38.7, 38.4, 38.2, 37.1, 37.0, 34.4, 32.0, 29.7, 28.0, 27.4, 27.2, 23.9, 20.9, 18.3, 18.1, 16.1, 15.9, 15.4, 14.6.

### Example 25

### Methyl platanate

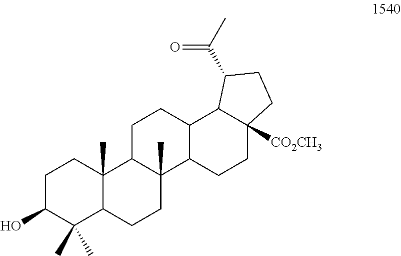

A solution of methyl betulinate (1.00 g, 2.13 mmol) in 9:1 DCM:MeOH (100 mL) was cooled to −78° C. and ozone was bubbled through until a faint blue colour was observed. The solution was purged with N₂ for 10 min and triethyl phosphite (1.80 mL, 10.5 mmol) was added. After 30 min, the solution was allowed to warm to rt overnight. The solvent was evaporated and the residue dissolved in EtOAc (100 mL), washed with water (40 mL) and brine (20 mL), dried (MgSO₄), filtered and solvent evaporated to give a white solid (1.35 g). Purification of the solid by silica gel chromatography using a hexanes/EtOAc gradient gave methyl platinate as a white powder (589 mg, 58%).

¹H NMR (CDCl₃, 400 MHz): δ (ppm) 3.67 (s, 3H), 3.28-3.22 (m, 1H), 3.18 (dd, J=11.2, 4.8 Hz, 1H), 2.27-2.22 (m, 1H), 2.17 (s, 3H), 0.98 (s, 3H), 0.95 (s, 3H), 0.88 (s, 3H), 0.81 (s, 3H), 0.75 (s, 3H).

¹³C NMR (CDCl₃, 400 MHz): δ (ppm) 212.4, 176.5, 78.9, 56.4, 55.3, 51.4, 51.2, 50.4, 49.4, 42.2, 40.5, 38.8, 38.6, 37.3, 37.2, 36.6, 34.2, 31.4, 30.1, 29.7, 28.3, 28.0, 27.3, 27.2, 20.9, 18.2, 16.1, 15.9, 15.3, 14.7.

### Example 26

### Methyl 3β,20-dihydroxy-lup-28-oates

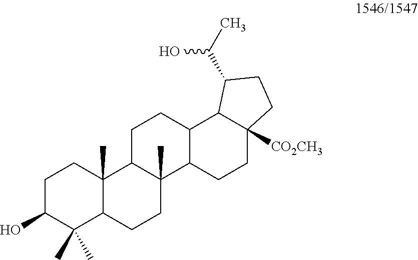

To a stirred solution of methyl platanate (164 mg, 0.348 mmol) in MeOH (8.5 mL) and DCM (3.5 mL) was added NaBH₄ (197 mg, 5.21 mmol) at rt. After 2 hrs, it was acidified with 30% HCl, stirred 15 min and solvent evaporated. The residue was diluted with water (20 mL) and extracted with DCM (3×20 mL). The combined organic extracts were washed with brine (2×20 mL), dried (MgSO₄), filtered and the solvent evaporated to give a white foam (162 mg). Separation of the foam by silica gel chromatography using a hexanes/EtOAc gradient separated the tow isomers. The less polar isomer was obtained in 47% yield as a white powder (Rf=0.25 in 2:3 EtOAc:hexanes) and the more polar isomer, also a white powder in 31% yield (Rf=0.14 in 2:3 EtOAc:hexanes).

Less polar isomer: ¹H NMR (CDCl₃, 400 MHz): δ (ppm) 3.89 (br q, J=6.3 Hz, 1H), 3.65 (s, 3H), 3.19 (dd, J=11.3, 5.0 Hz, 1H), 1.79 (dd, J=12.2, 7.2 Hz, 1H), 1.13 (d, J=6.4 Hz, 3H), 0.96 (s, 6H), 0.90 (s, 3H), 0.82 (s, 3H), 0.75 (s, 3H). ¹³C NMR (CDCl₃, 400 MHz): (ppm) 176.9, 78.9, 68.9, 57.0, 55.3, 51.2, 50.2, 48.0, 45.6, 42.4, 40.6, 38.8, 38.7, 38.0, 37.1, 37.0, 34.3, 31.7, 29.7, 28.0, 27.4, 27.1, 23.3, 22.2, 20.9, 18.3, 16.1, 15.9, 15.4, 14.7.

More polar isomer: ¹H NMR (CDCl₃, 400 MHz): δ (ppm) 4.03 (qd, J=6.3, 4.1 Hz, 1H), 3.65 (s, 3H), 3.19 (dd, J=11.3, 5.0 Hz, 1H), 2.55 (tt, J=10.6, 3.9 Hz, 1H), 2.26-2.17 (m, 2H), 1.85 (dd, J=12.4, 7.2 Hz, 1H), 1.07 (d, J=6.3 Hz, 3H), 0.96 (s, 3H), 0.91 (s, 3H), 0.90 (s, 3H), 0.82 (s, 3H), 0.75 (s, 3H).

¹³C NMR (CDCl₃, 400 MHz): δ (ppm) 176.5, 79.0, 69.7, 57.0, 55.3, 51.3, 50.3, 49.6, 45.7, 42.4, 40.7, 38.9, 38.7, 37.7, 37.20, 37.15, 34.3, 31.9, 29.5, 28.0, 27.3, 27.2, 22.9, 20.9, 18.3, 16.4, 16.1, 15.9, 15.4, 14.5.

HRMS: [M]+ not found; Calculated for [M-H₂O]⁺, C₃₀H₄₈O₃, 456.3604. Found [M-H₂O]⁺ 456.3635.

### Example 27

### 3-Benzoyl-ursolic Acid

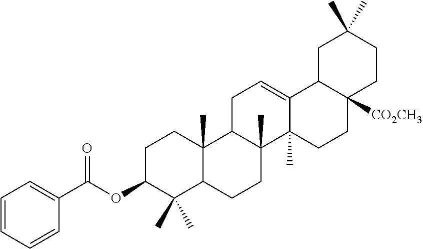

Crude ursolic acid isolated from apple peels (561 mg, ˜1.23 mmol) in pyridine (10 mL) was added DMAP (5 mg) and benzoic anhydride (1.39 g, 6.14 mmol). The mixture was stirred for 3 days at rt, then acidified to pH ˜4 with 10% HCl and stirred 30 min. The mixture was extracted with EtOAc (100 mL), washed with 5% HCl (40 mL), water (40 mL) and brine (40 mL), dried (MgSO₄), filtered and the solvent evaporated. The residue was purified using silica gel chromatography with a hexanes/EtOAc gradient to give a white solid containing benzoic acid. Heating the solid under vacuum removed benzoic acid by sublimation to give the desired product as a white powder (216 mg, 31%) containing 8% 3-benzoyl-oleanolic acid.

¹H NMR (CDCl₃, 400 MHz): δ (ppm) 8.07-8.03 (m, 2H), 7.57-7.53 (m, 1H), 7.46-7.42 (m, 2H), 5.25 (t, J=3.4 Hz, 1H), 4.75 (dd, J=10.9, 5.6 Hz, 1H), 2.19 (d, J=11.4 Hz, 1H), 1.10 (s, 3H), 1.024 (s, 3H), 1.018 (s, 3H), 0.96 (d, J=4.8 Hz, 3H), 0.95 (s, 3H), 0.88 (d, J=6.4 Hz, 3H), 0.80 (s, 3H).

¹³C NMR (CDCl₃, 400 MHz): δ (ppm) 183.7, 166.3, 137.9, 132.7, 131.0, 129.5, 128.3, 125.7, 81.6, 55.4, 52.5, 48.0, 47.5, 41.9, 39.5, 39.0, 38.8, 38.3, 38.1, 37.0, 36.7, 32.8, 30.6, 29.7, 28.2, 28.0, 24.0, 23.6, 23.3, 21.2, 18.2, 17.1, 17.02, 16.98, 15.5.

HRMS: Found no [M]^{+.}; Calculated for [M-HCO₂H]+ as C₃₆H₅₀O₂, 514.3811. found [M-HCO₂H]^{+.} 514.3815.

### Example 28

### Methyl 2α-hydroxy-3-oxo-urs-12-en-28-oate

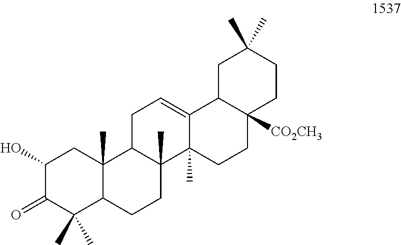

To a stirred solution of methyl 3-oxo-ursolate (300 mg, 0.64 mmol) in dry DCM (13.1 mL) under N₂ at −78° C. was added dry Et₃N (0.89 mL, 6.4 mmol) followed by TMSOTf (0.58 mL, 3.2 mmol). After 1 hr, the reaction was quenched with saturated NaHCO₃ solution (6 mL) and warmed to rt. Layers were separated and the aqueous layer extracted with hexanes (3×5 mL). The combined organic extracts were washed with brine (8 mL), dried (MgSO₄), filtered and the solvent evaporated to give the crude silyl enol ethers as white foam (347 mg).

The crude silyl enol ethers were dissolved in DCM (12 mL) and cooled to 0° C. To this was slowly added a solution of ˜77% mCPBA (158 mg 0.70 mmol) in DCM (15 mL), pre-cooled to 0° C. After 30 min, saturated NaHCO₃ solution (20 mL) was added and stirred 1 hr at rt. Layers were separated and the organic layer evaporated, giving silyl epoxides which were dissolved in MeOH (20 mL) with 5% HCl (1 mL) and stirred 45 min at rt. The solvent was evaporated and the residue dissolved in EtOAc (50 mL). It was washed with water (15 mL) and brine (15 mL), dried (MgSO₄), filtered and the solvent evaporated to give a colourless oil (346 mg). The oil was separated by silica gel chromatography using a hexanes/EtOAc gradient to give mainly the title compound contaminated with of the corresponding 11% oleanolate analog.

¹H NMR (CDCl₃, 400 MHz): δ (ppm) 5.26-5.24 (m, 1H), 4.54 (dd, J=12.6, 6.6 Hz, 1H), 3.61 (s, 3H), 2.43 (dd, J=12.5, 6.6 Hz, 1H), 2.24 (d, J=11.5 Hz, 1H), 1.27 (s, 3H), 1.16 (s, 3H), 1.11 (s, 3H), 1.05 (s, 3H), 0.93 (d, J=6.3 Hz, 3H), 0.84 (d, J=6.5 Hz, 3H), 0.80 (s, 3H).

¹³C NMR (CDCl₃, 400 MHz): δ (ppm) 216.7, 178.0, 138.4, 125.0, 69.2, 57.7, 52.7, 51.5, 49.6, 48.0, 47.7, 47.2, 42.0, 39.6, 39.0, 38.8, 37.6, 36.6, 32.6, 30.6, 28.0, 24.7, 24.1, 23.6, 23.5, 21.6, 21.2, 19.1, 17.1, 17.0, 16.1.

HRMS: Calculated for C₃₁H₄₈O₄, 484.3530. Found 484.3553.

### Example 29

### Methyl 2α-hydroxyursolate and methyl 2β-hydroxyursolate

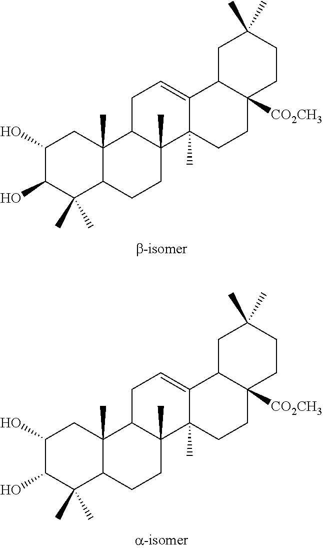

To a stirred solution of product in Example 27 (156 mg, 0.32 mmol) in MeOH (8 mL) at 0° C. was added NaBH₄ (25 mg, 0.66 mmol). After 1 hr, it was acidified with 30% HCl and the solvent was evaporated. The residue was diluted with water (10 mL) and extracted with EtOAc (3×8 mL). The combined extracts were washed with brine (8 mL), dried (MgSO₄), filtered and the solvent evaporated to give a white foam (149 mg). Separation of the foam by silica gel chromatography using a hexanes/EtOAc gradient gave the β-isomer as white powder (87 mg, 56%) and the 22 mg (14%) of the α-isomer. Each sample contained about 11% of the corresponding oleanolate derivative reflecting the purity of the ursolic acid used to prepare the precursors to these compounds.

β-Isomer: ¹H NMR (CDCl₃, 400 MHz): δ (ppm) 5.25 (t, J=3.5 Hz, 1H), 3.72-3.66 (m, 1H), 3.60 (s, 3H), 3.00 (d, J=9.5 Hz, 1H), 2.23 (d, J=11.3 Hz, 1H), 1.07 (s, 3H), 1.03 (s, 3H), 0.99 (s, 3H), 0.94 (d, J=5.8 Hz, 3H), 0.85 (d, J=6.4 Hz, 3H), 0.82 (s, 3H), 0.74 (s, 3H). ¹³C NMR (CDCl₃, 400 MHz): δ (ppm) 178.0, 138.2, 125.3, 84.0, 69.0, 55.3, 52.8, 51.5, 48.0, 47.5, 46.6, 42.0, 39.5, 39.1, 39.0, 38.8, 38.2, 36.6, 32.8, 30.6, 28.6, 27.9, 24.2, 23.6, 23.3, 21.2, 18.3, 17.0, 16.9, 16.8, 16.7.

α-Isomer: ¹H NMR (CDCl₃, 400 MHz): δ (ppm) 5.24 (t, J=3.5 Hz, 1H), 4.02-3.97 (m, 1H), 3.60 (s, 3H), 3.42 (d, J=2.5 Hz, 1H), 2.22 (d, J=11.2 Hz, 1H), 1.08 (s, 3H), 1.01 (s, 3H), 0.96 (s, 3H), 0.93 (d, J=6.0 Hz, 3H), 0.853 (s, 3H), 0.846 (d, J=6.2 Hz, 3H), 0.72 (s, 3H). ¹³C NMR (CDCl₃, 400 MHz): δ (ppm) 178.1, 138.3, 125.4, 78.9, 66.5, 52.8, 51.5, 48.09, 48.08, 47.3, 42.1, 41.9, 39.7, 39.0, 38.9, 38.3, 38.2, 36.6, 32.7, 30.7, 28.5, 28.0, 24.2, 23.8, 23.3, 21.9, 21.2, 18.0, 17.0, 16.9, 16.5.

### Example 30

### Preparation of Canophyllic Acid and Derivatives

Derivatives of this triterpene were prepared from canophyllol or canophyllic acid using known literature procedures: their spectroscopic properties were compared to literature values where available. Canophyllol was isolated from the bark of Ruptiliocaron caracolitone. Canophyllic acid was prepared by oxidation of canopyllol.

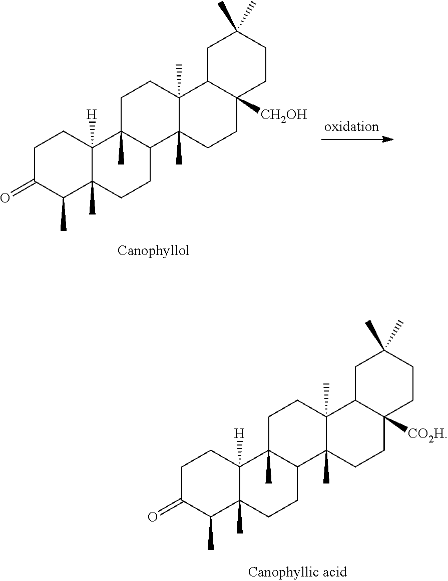

### Example 31

### Compounds of the Disclosure

The Table below provides further information for some of the compounds synthesized using the examples of the disclosure.

| | | Chemical Formula/ | | Common |
|---|---|---|---|---|
| CODE | Structure | Molecular Weight | Weight | Name |
| 1500 | 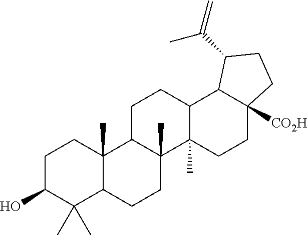 | Chemical Formula: C₃₀H₄₈O₃ Molecular Weight: 456.70032 | tan powder | Betulinic Acid |
| 1501 | 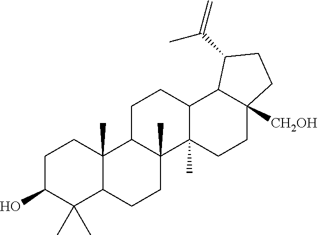 | Chemical Formula: C₃₀H₅₀O₂ Molecular Weight: 442.71680 | white powder | Betulin |
| 1502 | 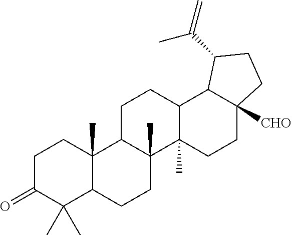 | Chemical Formula: C₃₀H₄₆O₂ Molecular Weight: 438.68504 | white powder | Betulon Aldehyde |
| 1503 | 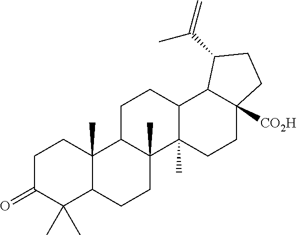 | Chemical Formula: C₃₀H₄₆O₃ Molecular Weight: 454.68444 | white powder | Betulonic Acid |
| 1504 | 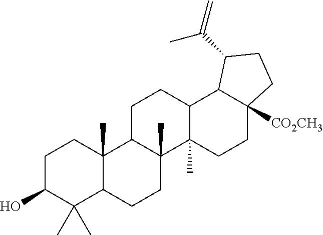 | Chemical Formula: C₃₁H₅₀O₃ Molecular Weight: 470.72690 | tan powder | Methyl Betulinate |
| 1505 | 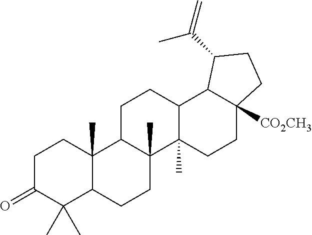 | Chemical Formula: C₃₁H₄₈O₃ Molecular Weight: 468.71102 | white powder | Methyl Betulonate |
| 1506 | 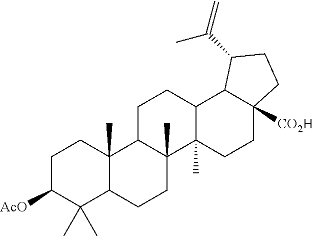 | Chemical Formula: C₃₂H₅₀O₄ Molecular Weight: 498.73700 | white powder | 3-acetoxy betulinic acid |
| 1507 | 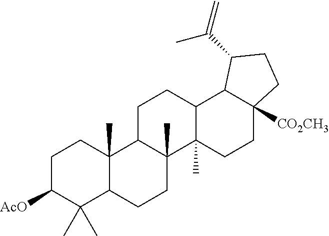 | Chemical Formula: C₃₃H₅₂O₄ Molecular Weight: 512.76358 | white powder | 3-acetoxy methyl betulinate |
| 1508 | 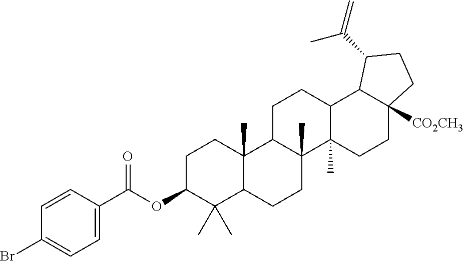 | Chemical Formula: C₃₈H₅₃BrO₄ Molecular Weight: 653.72902 | white powder | |
| 1509 | 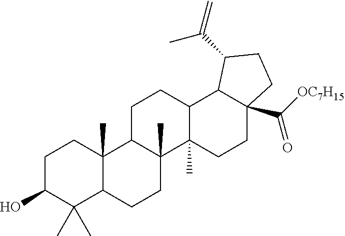 | Chemical Formula: C₃₇H₆₂O₃ Molecular Weight: 554.88638 | white powder | Heptyl Betulinate |
| 1510 | 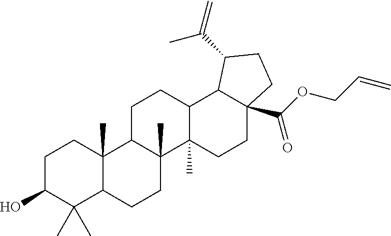 | Chemical Formula: C₃₃H₅₂O₃ Molecular Weight: 496.76418 | ~40 mg white powder | Allyl Betulinate |
| 1511 | 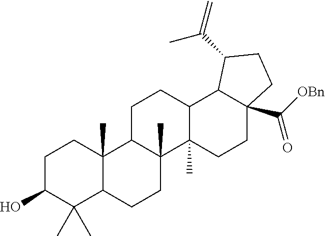 | Chemical Formula: C₃₇H₅₄O₃ Molecular Weight: 546.82286 | white crystals | Benzyl Betulinate |
| 1512 | 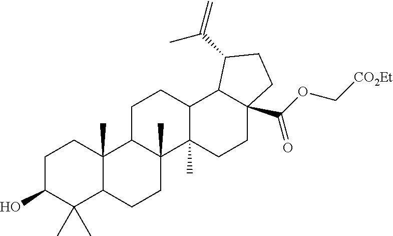 | Chemical Formula: C₃₄H₅₄O₅ Molecular Weight: 542.78956 | white powder | |
| 1513 | 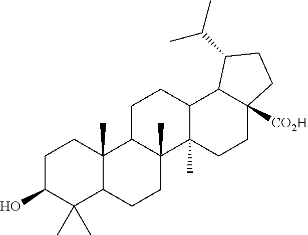 | Chemical Formula: C₃₀H₅₀O₃ Molecular Weight: 458.71620 | white needles | Dihydro- betulinic Acid |
| 1514 | 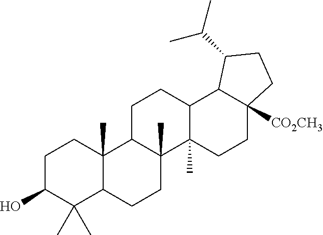 | Chemical Formula: C₃₁H₅₂O₃ Molecular Weight: 472.74278 | white powder | |
| 1515 | 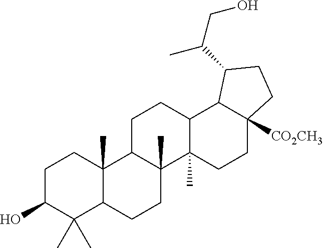 | Chemical Formula: C₃₁H₅₂O₄ Molecular Weight: 488.74218 | white flakes | |
| 1516 | 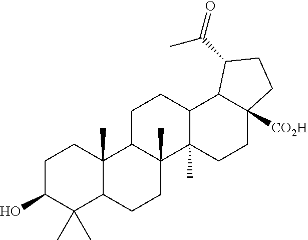 | Chemical Formula: C₂₉H₄₆O₄ Molecular Weight: 458.67314 | white powder | Platanic Acid |
| 1517 | 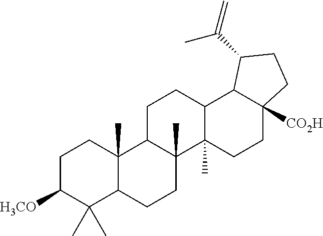 | Chemical Formula: C₃₁H₅₀O₃ Molecular Weight: 470.72690 | white powder | |
| 1518 | 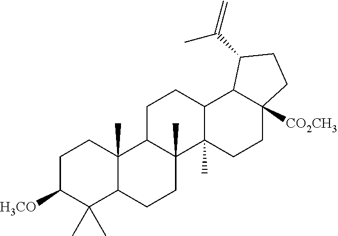 | Chemical Formula: C₃₂H₅₂O₃ Molecular Weight: 484.75348 | white powder | |
| 1519 | 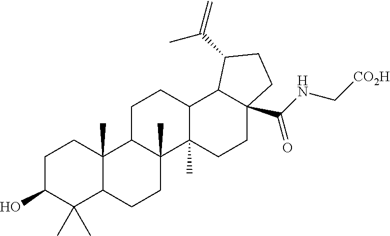 | Chemical Formula: C₃₂H₅₁NO₄ Molecular Weight: 513.75164 | white powder | |
| 1520 | 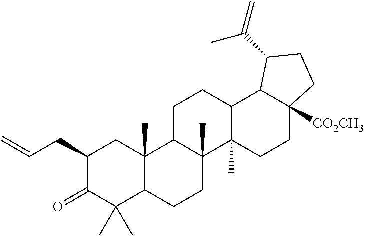 | Chemical Formula: C₃₄H₅₂O₃ Molecular Weight: 508.77488 | white foam | |
| 1521 | 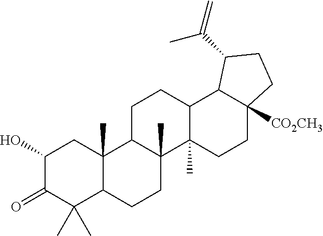 | Chemical Formula: C₃₁H₄₈O₄ Molecular Weight: 484.71042 | white foam | |
| 1522 | 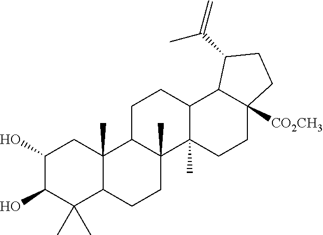 | Chemical Formula: C₃₁H₅₀O₄ Molecular Weight: 486.72630 | white foam | 2-α,3- β-Methyl Alphitolate |
| 1523 | 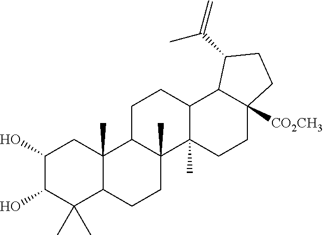 | Chemical Formula: C₃₁H₅₀O₄ Molecular Weight: 486.72630 | white foam | 2-α,3- β-Methyl Alphitolate |
| 1524 | 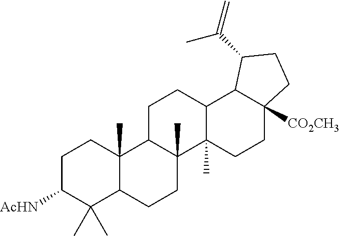 | Chemical Formula: C₃₃H₅₃NO₃ Molecular Weight: 511.77882 | white powder | |
| 1525 | 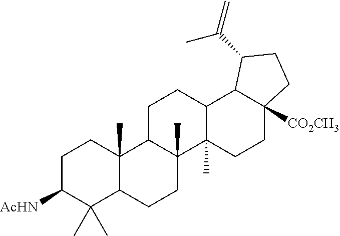 | Chemical Formula: C₃₃H₅₃NO₃ Molecular Weight: 511.77882 | white flakes | |
| 1526 | 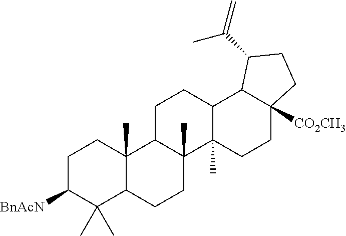 | Chemical Formula: C₄₀H₅₉NO₃ Molecular Weight: 601.90136 | white powder | |
| 1527 | 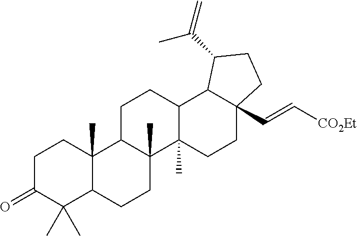 | Chemical Formula: C₃₄H₅₂O₃ Molecular Weight: 508.77488 | white foam | |
| 1528 | 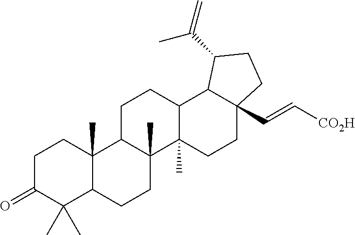 | Chemical Formula: C₃₂H₄₈O₃ Molecular Weight: 480.72172 | white powder | |
| 1529 | 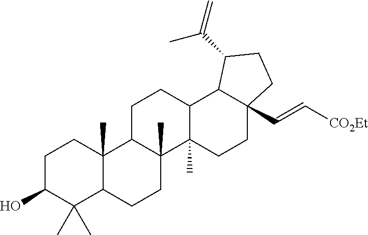 | Chemical Formula: C₃₄H₅₄O₃ Molecular Weight: 510.79076 | white powder | |
| 1530 | 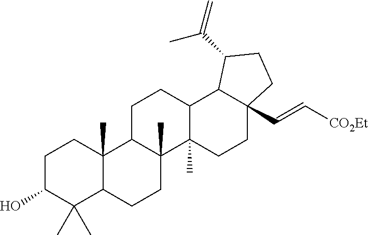 | Chemical Formula: C₃₄H₅₄O₃ Molecular Weight: 510.79076 | white foam | |
| 1531 | 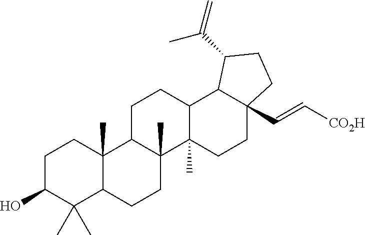 | Chemical Formula: C₃₂H₅₀O₃ Molecular Weight: 482.73760 | white powder | |
| 1532 | 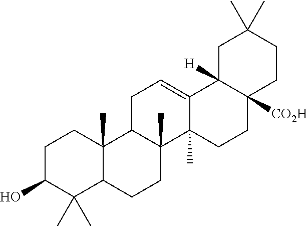 | Chemical Formula: C₃₀H₄₈O₃ Molecular Weight: 456.70032 | white powder | Oleanolic Acid |
| 1533 | 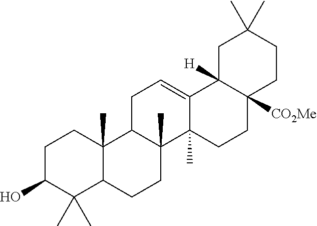 | Chemical Formula: C₃₁H₅₀O₃ Molecular Weight: 470.72690 | white powder | Methyl Oleanolate |
| 1534 | 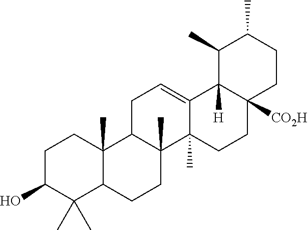 | Chemical Formula: C₃₀H₄₈O₃ Molecular Weight: 456.70032 | white powder | Ursolic Acid |
| 1535 | 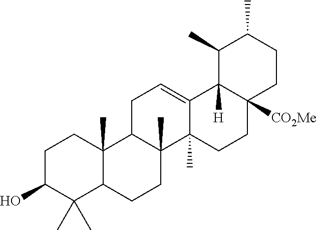 | Chemical Formula: C₃₁H₅₀O₃ Molecular Weight: 470.72690 | fluffy white needles | Methyl Ursolate |
| 1536 | 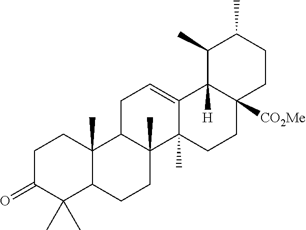 | Chemical Formula: C₃₁H₄₈O₃ Molecular Weight: 468.71102 | white powder | |
| 1537 | 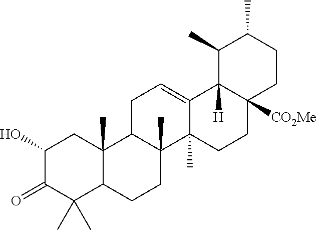 | Chemical Formula: C₃₁H₄₈O₄ Molecular Weight: 484.71042 | sticky white solid | |
| 1538 | 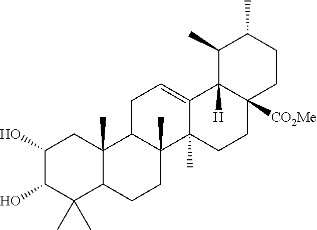 | Chemical Formula: C₃₁H₅₀O₄ Molecular Weight: 486.72630 | white powder | |
| 1539 | 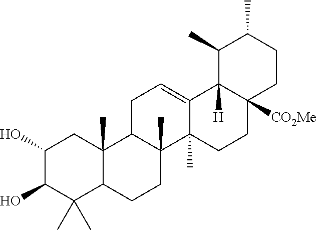 | Chemical Formula: C₃₁H₅₀O₄ Molecular Weight: 486.72630 | white powder | |
| 1540 | 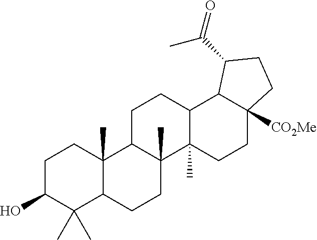 | Chemical Formula: C₃₀H₄₈O₄ Molecular Weight: 472.69972 | white powder | Methyl Platanate |
| 1541 | 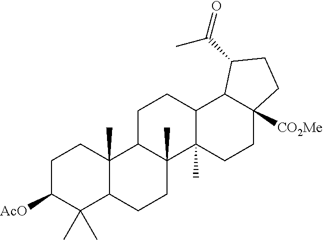 | Chemical Formula: C₃₂H₅₀O₅ Molecular Weight: 514.73640 | white powder | |
| 1542 | 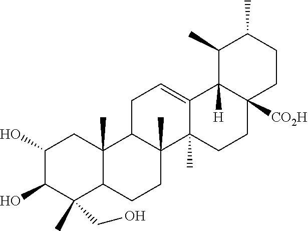 | Chemical Formula: C₃₀H₄₈O₅ Molecular Weight: 488.69912 | white powder | Asiatic Acid |
| 1543 | 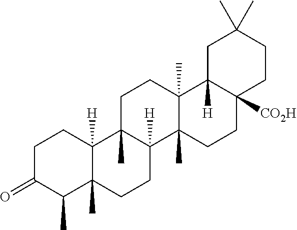 | Chemical Formula: C₃₀H₄₈O₃ Molecular Weight: 456.70032 | white powder | |
| 1544 | 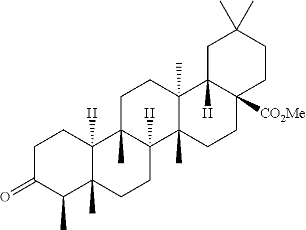 | Chemical Formula: C₃₁H₅₀O₃ Molecular Weight: 470.72690 | white powder | |
| 1545 | 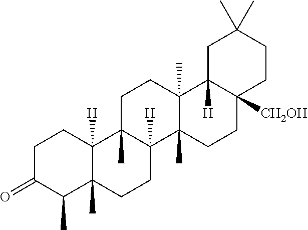 | Chemical Formula: C₃₀H₅₀O₂ Molecular Weight: 442.71680 | white powder | Canophyllol |
| 1546 | 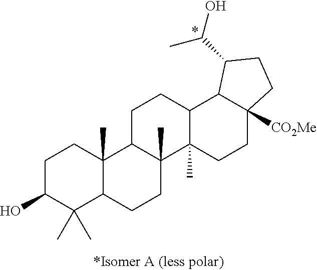 | Chemical Formula: C₃₀H₅₀O₄ Molecular Weight: 474.71560 | white powder | |
| 1547 | 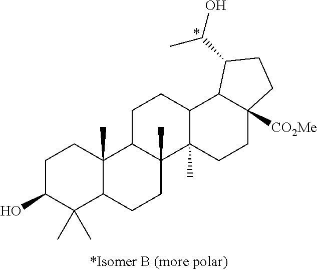 | Chemical Formula: C₃₀H₅₀O₄ Molecular Weight: 474.71560 | white powder | |
| 1548 | 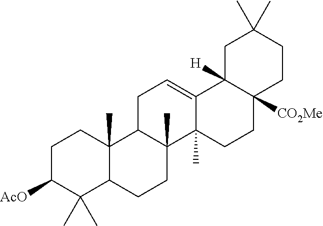 | Chemical Formula: C₃₃H₅₂O₄ Molecular Weight: 512.76358 | white powder | 3-acetoxy methyl oleanolate |
| 1549 | 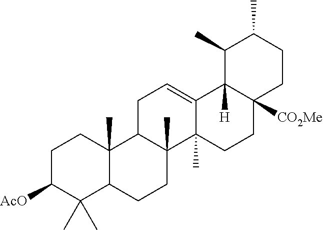 | Chemical Formula: C₃₃H₅₂O₄ Molecular Weight: 512.76358 | white powder | 3-acetoxy methyl ursolate |
| 1550 | 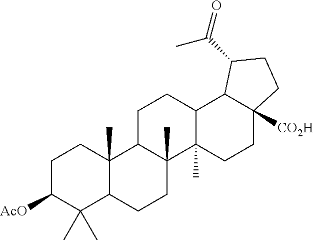 | Chemical Formula: C₃₁H₄₈O₅ Molecular Weight: 500.70982 | white powder | 3-acetoxy platanic acid |

### Example 32

### Evaluation of Cortisol Lowering Activity

Trout head kidney cells were prepared as adapted from Leblond et al. (Leblond et al., 2001). Briefly, fish were anesthetized with benzocaine (30 to 35 mg/L), blood was collected by caudal puncture into heparinized syringes and fish were then euthanized by a sharp blow to the head followed by trans-spinal sectioning. The head kidney was removed and placed in a solution of enriched minimum essential media (MEM, Sigma-Aldrich) supplemented with collagenase/dispase (2 mg/mL). The tissue was manually disrupted by gently pressing it on the interior walls of the tube with a small spatula and incubated for 1 h at 10-13° C. with shaking. The medium was then filtered (200 μm filter, rinsed with MEM, then a 75 μm filter and rinsed with MEM), centrifuged (260 g, 10-13° C., 5 min) and the pellet resuspended in 1 mL MEM.

Cells were plated in a 96-well microtiter plate with 150 μL containing 50×10⁶ cells/mL. Cells were incubated with BA analogues (60 μg/mL) dissolved in ethanol and DMSO (final concentration of DMSO: 0.3% v/v, ethanol: 1% v/v) for 60 min at 10-13° C., then stimulated with 1 U/mL ACTH (Sigma-Aldrich) and incubated for an additional 60 min. Then cells were collected and transferred to 1.5 mL conical plastic tubes and centrifuged (20,000×g, 2 min); the supernatant was collected and flash frozen in liquid nitrogen for subsequent cortisol assay using a commercially available, standard RIA (MP Biomedicals Ltd., Solon, ON). The cells were assessed for cytotoxicity at the end of the experiment with the lactate dehydrogenase (LDH) assay according to Mommsen and Moon (Mommsen and Moon, 1987). Four control wells were prepared: DMSO/ethanol blank (0.3% v/vDMSO, 1% v/v ethanol), DMSO/ethanol plus ACTH (1 U/mL), MEM blank and MEM plus ACTH (1 U/mL).

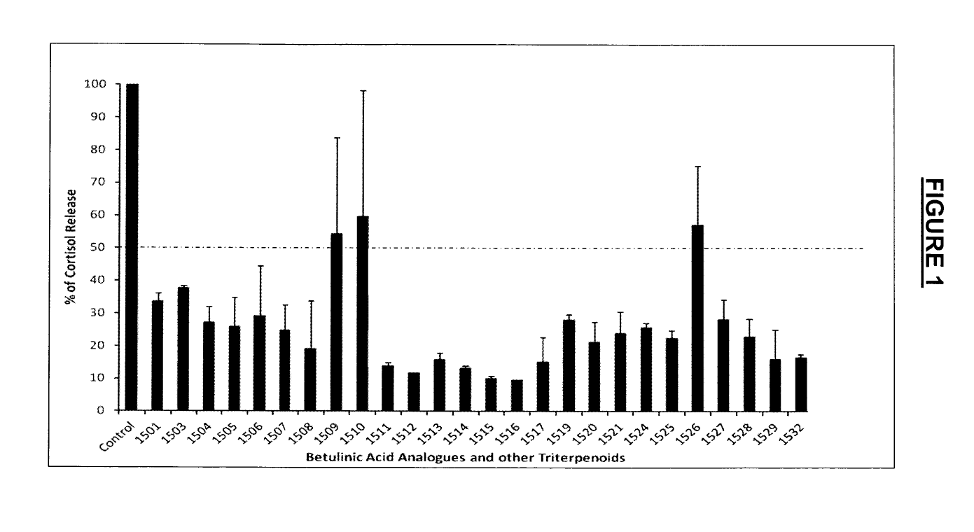

Rainbow trout head kidney cells pre-incubated with compounds of the disclosure (60 μg/mL) released less cortisol in response to an ACTH (1 U/mL) challenge than the positive control group (MEM+ACTH, DMSO/ethanol) (FIG. 1). Incubation with the compounds had no significant effect on cell viability (as assessed by LDH leakage) in the assay as compared to the negative (un-stimulated) or positive (0, MEM+ACTH) controls.

The results of the assay are shown in FIG. 1, which shows the mean cortisol release, as compared to control, for head kidney cells incubated with triterpene acid analogs (60 μg/mL) following an ACTH challenge (1 U/mL). The dashed line represents a 50% reduction in cortisol response, as compared to the control.

### Example 33

### Treatment of Dyslipidemia and Weight Control in Mice

In this example, mice fed with high fat diet are administered compounds of the formula (I) and show a decrease in cortisol concomitant with improvement in fatty acid profile. The effect of compounds of the formula (I) on cortisol levels, fatty acid levels and weight is examined using 10-week female C57BL/6 mice (Jackson Laboratory, Bar Harbor, Me., USA.) that are initially maintained on normal rodent diet and then fed on the atherogenic diet for 12 weeks as described in Li et al. (2008). Mice are divided into three groups that are fed a normal diet, or switched to a high fat diet or atherogenic diet. Mice in each group are further divided into two sub-groups, and each diet is either supplemented with or without compounds of the formula (I). Mice are weighed before the switch in diet and then at weeks, 4, 8 and 12. Blood samples are taken before the change in diet and then at weeks 4, 8 and 12. The plasma lipid levels, glucose and other parameters are measured as described in Li et al. (2008). Mice which have had compounds of the formula (I) administered show an improvement in the fatty acid profile, lower hypercholesterolemia and a decrease in weight, in comparison with mice with non-administered diets.

### Example 34

### Evaluation of Cortisol Lowering Activity in Trout Head Kidney Slice Model

Fish—

Female juvenile rainbow trout (Oncorhynchus mykiss), 100-300 g, were obtained from Linwood Acres Trout Farm (Campellcroft, ON) and were acclimated for a minimum of two weeks in the uOttawa Aquatic Care Facility. Fish were maintained in circular fiberglass holding tanks (1200 L) continuously supplied with aerated, dechloraminated City of Ottawa tap water at 13° C. and kept at a constant 12:12 light-dark photoperiod. Trout were fed at 0.5% body weight daily with a commercial diet (Classic Floating Trout Grower, Martin Mills, Tavistock, ON). This represents maintenance, not a growth ration for these animals.

Head Kidney Tissue Preparation—

Head kidney tissue was isolated using a modified method as described by Conde-Sieira et al. (2013). Fish were anaesthetized with benzocaine (30-35 mg/L), blood was removed from the caudal vein using a 3 mL heparinized syringe and the fish euthanized by spinal transection. The vasculature was perfused through the post-cardinal vein with 40 mL of modified Hanks' medium (92.56 mM NaCl, 3.63 mM KCl, 0.55 mM MgSO₄, 0.4 mM KH₂PO₄, 0.23 Na₂HPO₄, 7.5 mM Hepes, 2.81 mM NaHCO₃, 0.85 mM CaCl₂, 0.03% (w/v) bovine albumin serum) containing EGTA (1 mM final) adjusted to pH 7.64. The entire head kidney was removed from the trout and transferred to a 15 mL chilled glass petri dish containing modified Hanks' medium (pH 7.15). The tissue was weighed then transferred to a 35 mL chilled glass petri dish containing 50 mL modified Hanks' medium per gram of tissue supplemented with MEM amino acid solution (50×; 2 mL/100 mL medium; Sigma—M5550), MEM non-essential amino acid solution (100×; 1 mL/100 mL medium; Sigma˜M7145) and Antibiotic-Antimycotic (100×; 1 mL/100 mL medium; Life Technologies˜15240). The tissue was chopped finely with a razor blade then separated further using a rubber policeman. The tissue was incubated at 13° C. with gentle agitation for 1.5 h to ensure basal levels of cortisol release. Optimal incubation times were determined in previous experiments (Conde-Sieira et al., 2013).

In Vitro Exposure of Compounds of the Disclosure—

Following the 1.5 h pre-incubation, tissue was placed in 48-well culture plates (approximately 25 mg tissue pre well) along with a final volume of 250 μL modified Hanks' medium. There were two wells for each compound treatment; one control (−ACTH) and one stimulated (+ACTH) that were conducted simultaneously. Tissues were incubated with compounds of the disclosure (60 μg/mL) initiated at the same time as determined in previous experiments (Mullally et al., 2012) for 60 min at 13° C. with gentle agitation and stimulated with 1 U/mL ACTH (Sigma-Aldrich). The compounds were dissolved in ethanol and DMSO (final concentration of DMSO: 0.3% v/v, ethanol 1% v/v) by vortexing for 1 min and sonication for 30 s if the compound was not fully dissolved (determined by visual inspection). After incubation, tissue and supernatant were collected and transferred to pre-weighed 1.5 mL conical plastic tubes and centrifuged (14 000 RCF, 10 min). The supernatant was collected and transferred into a new 1.5 mL conical plastic tube and flash frozen in liquid nitrogen for subsequent cortisol assay. The mass of tissue for each sample was determined by weighing the remaining tissue in the pre-weighed tubes. Four control wells were prepared for each preparation: modified Hanks' medium, modified Hanks' medium plus ACTH (1 U/mL), DMSO/ethanol blank (0.3% v/v DMSO, 1% v/v ethanol) and DMSO/ethanol blank plus ACTH (1 U/mL).

Cortisol Assay—

Cortisol concentrations were assessed using a ¹²⁵I RIA kit (MP Biomedicals, Irvine, Calif., USA) as per the manufacturer's instructions and read using a Packard® Cobra™ Auto-Gamma Counter. Counts were normalized to tissue weights in each experimental well.

Statistical Analysis—

Data are presented as % change in ACTH-stimulated cortisol release relative to control preparation (modified Hanks' medium stimulated with ACTH) using GraphPad Prism software (GraphPad Software, Inc., La Jolla, Calif.). Dose response curves were conducted for the compounds of the disclosure that statistically reduced cortisol levels. GraphPad Prism software (GraphPad Software, Inc., La Jolla, Calif.) was used to graph and calculate EC₅₀ values by fitting a sigmoidal curve to the dose response data as well as linear regressions between Log P of compounds of the disclosure vs log cortisol responses.

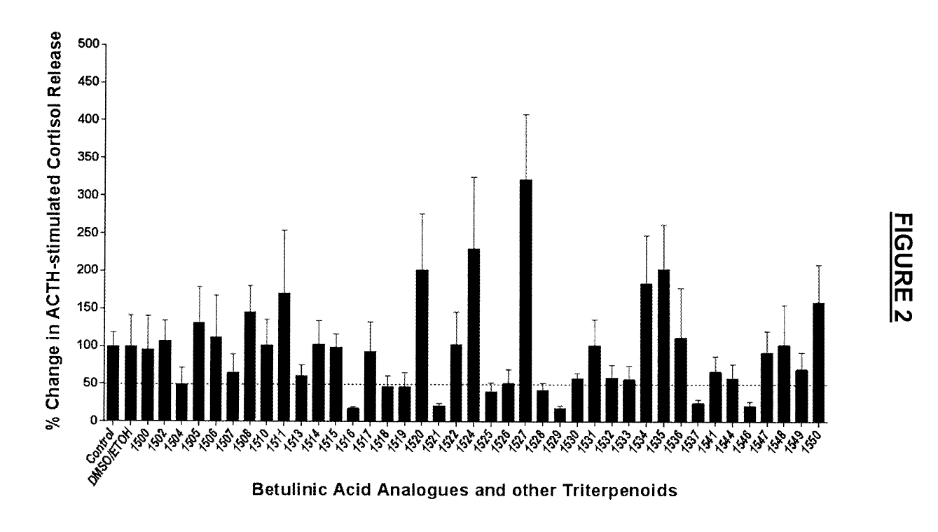

FIG. 2 is a bar graph showing the effect of the compounds of the disclosure on cortisol release, in particular, the percent change in ACTH-stimulated (1 U/ml) cortisol release as compared to control, for trout head kidney tissue incubated with compounds of the disclosure (60 μg/ml). Data are normalized to the same tissue incubated in modified Hanks' medium. Data represent means+SEM (n=3-7). T-tests were performed between each compound and their individual control preparation (modified Hanks' medium).

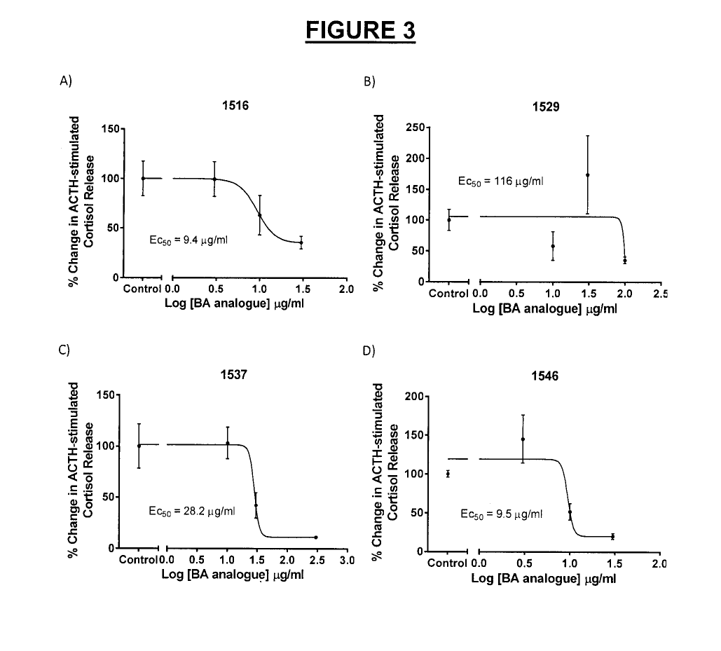

Compounds 1516, 1537 and 1546 inhibited cortisol production in a dose-dependent manner with calculated EC₅₀ values of approximately 9, 28 and 9 μg/ml, respectively (see FIG. 3). FIG. 3 shows the percent change in ACTH-stimulated cortisol release in trout head kidney tissues incubated with compounds 1516(A), 1529 (B), 1537 (C) and 1546 (D). Data represent mean±SEM (n=3-7). A four parameter sigmoidal curve was fitted to the data using GraphPad Prism and the EC₅₀ was calculated to be 9.4, 116, 28.2 and 9.5 μg/ml for compounds 1516, 1529, 1537 and 1546, respectively.

Compound 1546 stimulated at low concentrations, followed by inhibition at higher concentrations. This hormesis effect explains the stimulation at 60 μg/ml by several compounds and indicates they would be inhibitory at higher concentrations.

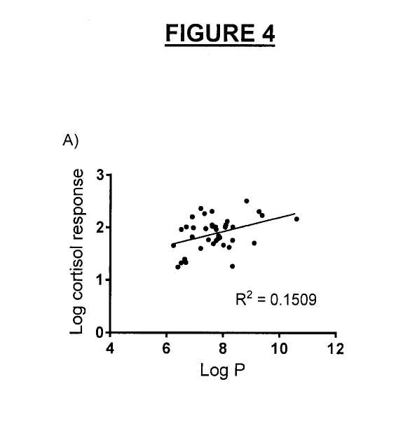

A correlation was shown between Log P (partition coefficient) and log cortisol response, as shown in FIG. 4.

Compounds 1516, 1521, 1529, 1537 and 1546, which lowered cortisol production, have relatively low Log Ps [<6.7] except 1529. This compares with betulinic acid with a calculated log P of 7.38.

### Example 35

### Anxiety and Cortisol Lowering Properties of Betulinic Acid and Amyrins

This example demonstrates that the combination of various compounds has a synergizing effect which lowers cortisol in animals.

The effect of Betulinic acid (BA), amyrins (alpha and beta) and the combination of the two were tested for (a) lowering corticosterone and (b) anxiety-like behavior and fear response, as tested with rats via Elevated Plus Maze and Contextual Conditioned Emotional Response tests.

### Materials and Methods

Rats—

All protocols were approved by the Animal Care Committee of the University of Ottawa. Male Sprague Dawley rats (n=32) were obtained from Charles River Laboratories (Rochefort, Quebec, Canada). They were individually housed in solid bottom plastic shoebox cages (47 cm×26 cm×21 cm) with the floors covered with Teklad Aspen Sani Chips bedding (Harlan Laboratories, Indianapolis, Ind.) and maintained on a 12-h:12-h light:dark cycle, with lights on at 7:00 a.m., with free access to water and standard rat chow. Each cage contained a plastic tube for enrichment. Room temperature was maintained at 21-23° C. with 60% relative humidity. The experimenter handled all the rats daily for 2-3 min throughout the experiment. During handling, the rat was removed from its cage and placed on the experimenter's arm, allowing the rat to move freely and explore and interact with the experimenter. Rats were orally administered for 5 days prior to assigned treatments a 50% solution of Eagle Brand sweetened condensed milk each day to familiarize them with the feeding procedure. All animals were habituated to the feeding of sweet and condensed milk technique. All animals were also habituated to the blood collection procedure once daily during the 3 days preceding the actual blood collection. They were transported to a room located a short distance from their home room, placed on a table and covered with a small towel, and their tails were then gently stroked for 1 min to mimic tail ‘milking’.

Oral Treatments of Rats—

All rats were orally administered the respective treatments for three consecutive days (2 days and 60 min prior to testing). One group of rats were treated with 2 mg/Kg of BA, the second group was treated with amyrins (50:50 mixture of both α-amyrin and β-amyrin) and the combination group treatment consisted of a 50:50 mixture of BA and amyrins.

Acute Mild Stress Restraint Procedure with Rats—

On blood collection day, 60 min post-treatment, rats were restrained by inserting them into a cone shaped disposable plastic restraint with nose hole (to facilitate respiration) for 2 min each after which the first of five blood sample was immediately taken.

Blood Collection and Sampling—

The tail vein venepuncture procedure was used to collect blood from the lateral tail vein of unanesthetized rats. This procedure does not impact measurements of corticosterone if samples are collected in under 3 min. Blood samples were collected from all animals between 10:00 h and 15:00 h. The tail was punctured on the tip of the tail using a 26 G needle and the end of the tail was milked to acquire blood. After wiping away any surfacing blood, two drops of blood (approximately 15-25 μl from each animal) were collected on Schleicher and Schuell specimen collection paper (Whatman International Ltd., Maidstone, UK). Enough blood was collected to completely soak through the collection paper and form a circular spot with a diameter larger than 3.0 mm. The drop paper samples were dried overnight at room temperature and then stored at −80° C. Five samples were taken immediately after restraint 0 min and 5, 10, 30 and 60 min after restraint. During the experiment, each rat received no more than two tail punctures, one after restraint and one 60 min after restraint.

Quantification of Corticosterone from Drop Samples of Rat Blood—

A 3.0 mm diameter circle was punched from the collection paper containing each drop sample, using a Gem Hole Punch (McGill Inc., Marengo, Ill.), and placed in a tube containing 200 μl of Dulbecco's phosphate-buffered saline (DPBS; Sigma-Aldrich, St. Louis, Mo.) containing 0.1% gelatin (Avantor Performance Materials, Phillipsburg, N.J.). The tubes were shaken in an orbital shaker at 90 r.p.m. for 1 h at 24° C. and then refrigerated for 48 h at 4° C. before the radioimmunoassay procedure. Corticosterone was quantified using a commercially available radioimmunoassay kit (Corticosterone Double Antibody 125I RIA Kit; MP Biomedicals, Solon, Ohio). We followed the manufacturer's protocol to prepare drop samples and standards. Standard and sample tubes were analyzed in a HP Cobra II gamma counter (Canberra-Packard, Meriden, Conn.). Corticosterone concentrations in the drop blood samples were quantified in units of pg corticosterone per punch.

Behavioral Paradigms

Elevated Plus Maze (EPM)

The EPM is a validated test used to assess anxiety-like behavior in laboratory rodents (Pellow and File, 1986; File 1995). The EPM consists of two open arms (planks; 50×10 cm), two perpendicular arms enclosed by 40 cm high walls (alleys), and is placed 50 cm above the ground. The EPM is based on the conflict between the animal's instinct to explore its environment and its fear of exposed (vulnerable to attack) areas and the height. Black curtains surrounded the chamber to limit the influence of spatial cues and other extraneous stimuli. A video camera was mounted above the arena to permit remote monitoring and recording. Rats were individually placed in the testing room for 1 h of acclimatization. Each rat was then placed onto the open central platform of the EPM (facing a closed arm). The rats behavior was monitored for 5 min and scored as follows: (1) percentage of time spent on the open arms (time open/300×100), (2) frequency of entries in the closed arms, and (3) unprotected head dips (UH); head protruding over the edge of an open arm and down toward the floor, which is an index of risk assessment behavior. Between tests, the EPM was cleaned with 70% isopropanol (to control for confounding residual odor). The percent of time in the open arms, and unprotected head dips are validated measures of anxiety-like behavior in the EPM which increases in these measures indicative of reduced anxiety. In contrast, the frequency of closed arm entries is an index of locomotor activity.

Conditioned Emotional Response (CER) Test

The conditioning chamber (Coulbourn Instruments) for assessment of CER measured 31 cm×25 cm×30 cm. The front and back walls were made of clear Plexiglas and the sidewalls were made of stainless steel panels. The floor was composed of 16 stainless steel rods (4 mm diameter, 1.4 cm apart), which were connected to a shock generator (Coulbourn Instruments, model H13-16) that delivered constant current.

All subjects completed one day of training followed by a day of testing 24 hours later. During the contextual training phase, subjects were placed in the conditioning chamber where they received 6 foot shocks (1.0 mA; 1 second in duration) with an average inter-trial interval (ITI) of 1 min. Cued fear training comprised the delivery of 6 pairings of a 20-s tone with a 1.0 mA (1-s) continuous footshock. The shock was delivered during the final second of the 20-s tone. Again, each trial was delivered at an average ITI of 1-min. On test days, contextual fear was assessed over a 15-minute or a 20 min period by placing them in the conditioning chamber where they had previously been shocked. Freezing behavior, as defined by the absence of movement excluding involuntary respiratory activity, was assessed. Evaluations were conducted by trained experimenters blind to the treatment group. To assess the CER (in the cued condition), rats were transferred to a novel environment of similar dimensions, but visually and textually distinct from the training chamber. Specifically, black laminate covered the walls, and the floor was smooth (instead of rod-grid floor) and covered with bedding chips. Animals were allowed a 1-min exploration period and were then presented with the conditioned cue (the tone that had previously been paired with footshock). A total of 15 tones (each 20-s in duration) were presented at 1-min intervals (20 s tone+40 s ITI). Freezing was scored as described in the contextual test. Between each training and testing session, cages were cleaned with 70% ethanol.

Statistical Analyses—

One-way and two-way analyses of variance (ANOVA) with Bonferonni studentized range tests were performed for mean comparisons (Zar, 1999). Kolmogorov-Schmirnoff and Levene's tests were used to verify the normality of distribution and the homogeneity of residual variance, respectively. All of the Fisher statistics (F), degrees of freedom (df), and p-value estimates were calculated with S-PLUS software version 7.0 (Insightful Corp., Seattle, Wash.). Data are reported as means±S.E.M and the level of significance was set at p<0.05. Linear regressions were conducted with Excel software.

Results and Discussion

Corticosterone

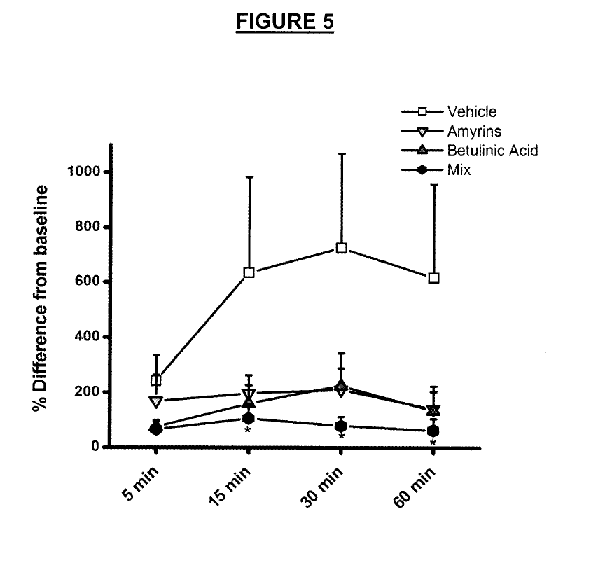

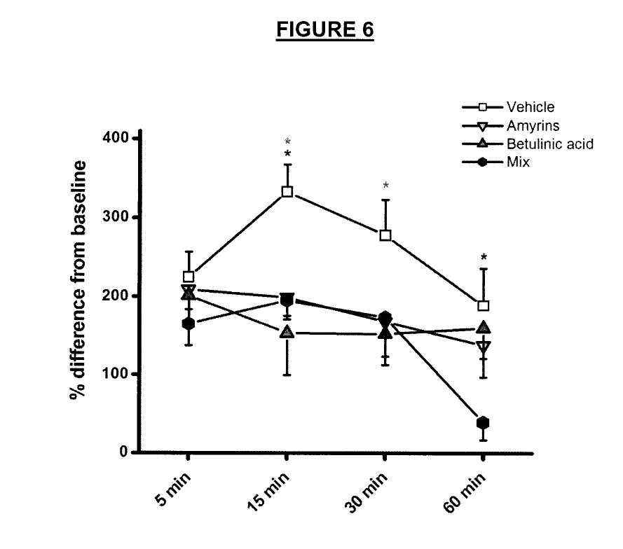

A rise in corticosterone was observed due to the restraint in the single dose study. When adjusted for baseline (FIG. 5), the mixture of amyrins and BA elicited a reduction in the corticosterone response at 15, 30 and 60 min. In the 3 day dosage study the relative differences compared to baseline (FIG. 6), shows that all 3 treatments were effective in reducing the corticosterone response at 15 min time point but as time passed, the mixture of both compounds was most effective in eliciting a reduction in the corticosterone response as seen in the 60 min test, indicating a synergistic response. FIG. 5 shows relative corticosterone values obtained from venepuncture blood samples collected from all rats at baseline and 5, 10, 15, 30 and 60 min after acute mild restraint (n=32) following a single oral administration of their respective treatments (*p<0.05 indicate significant differences from vehicle. There were no significant differences between any of the groups at any time point). FIG. 6 shows relative corticosterone values obtained from venepuncture blood samples collected from all rats at baseline and 5, 10, 15, 30 and 60 minutes after acute mild restraint (n=32) following 3 consecutive daily oral administration of their respective treatments (*p<0.05 indicate significant differences from vehicle).

The Effect of Amyrins and Betulinic Acid in Reducing Fear and Anxiety-Like Behaviors

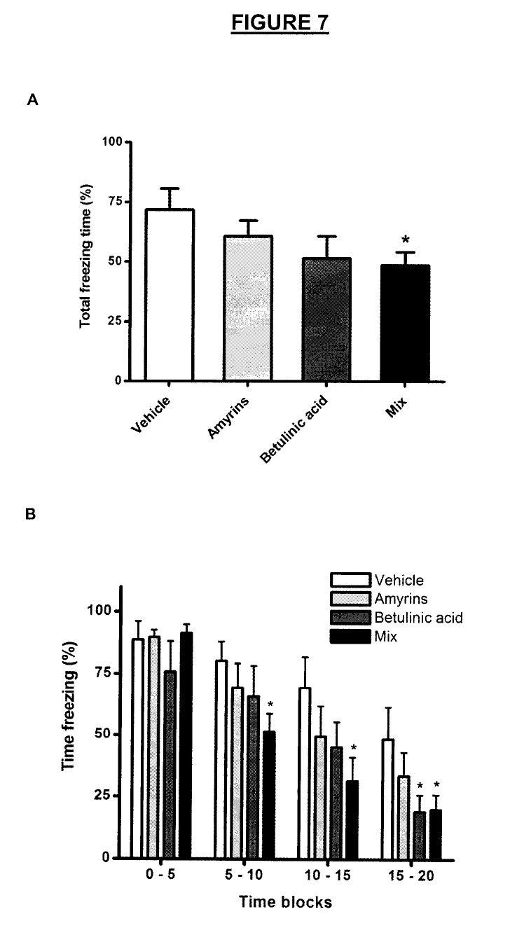

As depicted in FIG. 7, the mixture of BA and Amyrins reduces freezing behaviour (FIGS. 7a and 7b), to a greater extent that either the BA or Amryins alone. FIG. 7a shows the effects of isolated compounds on total freezing time (%) in rats in the contextual conditioned emotional response test following 3 day oral administration at 1 mg/kg of their respective treatments. The amyrins group consist of a 50:50 mixture of both α-amyrin and β-amyrin. The mix group consists of a 50:50 mixture of BA and amyrins. *P<0.05 indicates a significant difference from vehicle.

FIG. 7b shows the breakdown of the (%) freezing time in rats in the contextual conditioned emotional response test following 3 day oral administration at 1 mg/kg of their respective treatments. The amyrins group consist of a 50:50 mixture of both α-amyrin and β-amyrin. The mix group consists of a 50:50 mixture of BA and amyrins. *P<0.05 indicates a difference from vehicle.

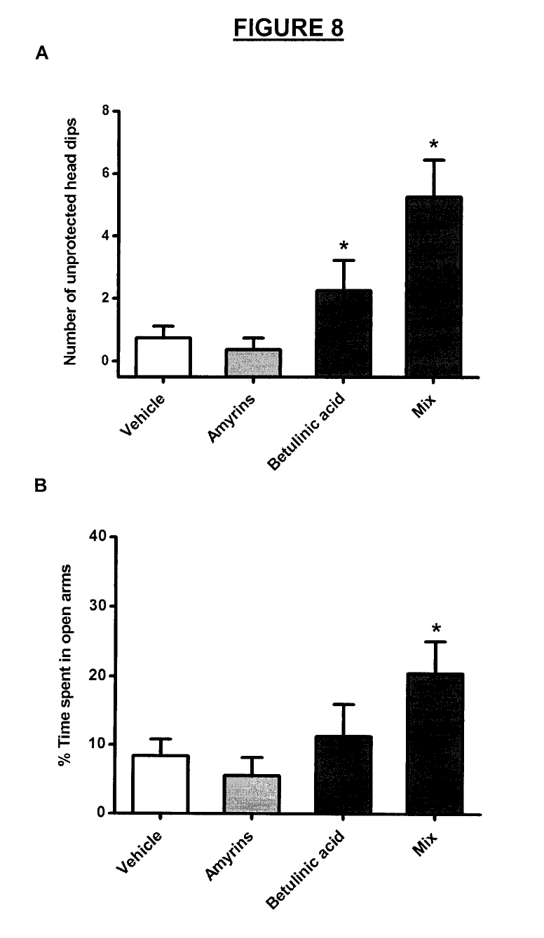

As depicted in FIG. 8 the mixture of BA and Amyrins induces increases in exploratory behaviour, compared to the compounds alone. FIG. 8a demonstrates the effects of isolated compounds on the percentage of time spent in the open arms in rats in the elevated plus maze following 3 day (1 mg/kg) oral administrations of their respective treatments. The amyrins group consist of a 50:50 mixture of both α-amyrin and β-amyrin. The mix group consists of a 50:50 mixture of BA and amyrins. *P<0.05 indicates a significant difference from vehicle. FIG. 8b shows the effects of isolated compounds on the number of unprotected head dips in rats in the elevated plus maze following 3 day (1 mg/kg) oral administrations of their respective treatments. The amyrins group consist of a 50:50 mixture of both α-amyrin and β-amyrin. The mix group consists of a 50:50 mixture of BA and amyrins. *P<0.05 indicates a significant difference from vehicle.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES CITED HEREIN

American Psychiatric Association. (1994). Diagnostic and statistical manual of mental disorders, 4^{th} Ed. (DSM-IV). Washington, D.C.
American Psychiatric Association: Diagnostic and Statistical Manual of Mental Disorders, Fifth Edition. Arlington, Va., American Psychiatric Association, 2013 (DSM-V).
Anagnostis, P., Athyros, V. G., Tziomalos, K., Karagiannis, A., Mikhailidis, D. P., 2009. The pathogenetic role of cortisol in the metabolic syndrome: A hypothesis. Journal of Clinical Endocrinology and Metabolism. 94, 2692-2701.
Brown D F, Brown D D (2003). USMLE Step 1 Secrets: Questions You Will Be Asked on USMLE Step 1. Philadelphia: Hanley & Belfus. p. 63. ISBN 1-56053-570-9
Brown, E., Bobadilla, L., Rush, A., 2001. Ketoconazole in bipolar patients with depressive symptoms: a case series and literature review 3, 23.
Carroll, B. J., Cassidy, F., Naftolowitz, D., Tatham, N. E., Wilson, W. H., Iranmanesh, A., Liu, P. Y., Veldhuis, J. D., 2007. Pathophysiology of hypercortisolism in depression. Acta Psychiatrica Scandinavica. 115, 90-103
Chandola, T., Brunner, E., Marmot, M., 2006. Chronic stress at work and the metabolic syndrome: Prospective study. British medical journal. 332, 521-524.
Cook, C. S., Berry, L. M., Burton, E., 2004. Prediction of in vivo drug interactions with eplerenone in man from in vitro metabolic inhibition data. Xenobiotica. 34.
Dallman, M. F. 2009. Stress-induced obesity and the emotional nervous system. Trends in Endocrinology and metabolism. 21, 159-165.
Edwards, J G (1991) Clinical anxiety and its treatment. Neuropeptides, 19 S1-10
Gallagher, P., Malik, N., Newham, J., Young, A. H., Ferrier, I. N., Mackin, P., 2008. Antiglucocorticoid treatments for mood disorders.
Gamperl, A. K., Vijayan, M. M., Boutilier, R. G., 1994. Experimental Control of Stress Hormone Levels in Fishes—Techniques and Applications. Reviews in Fish Biology and Fisheries. 4, 215-255.
Gathercole, L. L., Stewart, P. M., 2010. Targeting the pre-receptor metabolism of cortisol as a novel therapy in obesity and diabetes. Journal of Steroid Biochemistry and Molecular Biology. 122, 21-27.
Gray, J. A., & McNaughton, N. (2000). The Neuropsychology of Anxiety: An Enquiry into the Functions of the Septohippocampal System (2nd ed.) Oxford, UK: Oxford University Press.
Jahn, H., Schick, M., Kiefer, F., Kellner, M., Yassouridis, A., Wiedemann, K., 2004. Metyrapone as additive treatment in major depression: A double-blind and placebo-controlled trial. Archives of General Psychiatry. 61, 1235-1244.
Hajat, A., Diez-Roux, A., Franklin, T. G., Seeman, T., Shrager, S., Ranjit, N., Castro, C., Watson, K., Sanchez, B., Kirschbaum, C., 2010. Socioeconomic and race/ethnic differences in daily salivary cortisol profiles: The Multi-Ethnic Study of Atherosclerosis. Psychoneuroendocrinology. 35, 932-943.
Herron M E, Shofer F S, Reisner I R. Retrospective evaluation of the effects of diazepam in dogs with anxiety-related behaviour problems. J Am Vet Med Assoc. 2008 Nov. 1; 233(9):1420-4.
Kim, T.-., Kim, B.-., Kim, Y.-., Yang, D. M., Han, Y.-., Dong, S. H., Kim, H. J., Chang, Y.-., Lee, J. I., Chang, R., 2003b. Liver cirrhosis developed after ketoconazole-induced acute hepatic injury. Journal of gastroenterology and hepatology. 18, 1426-1429.
Lawson E A, Donoho D, Miller K K, Misra M, Meenaghan E, Lydecker J, Wexler T, Herzog D B, Klibanski A. 2009, J Clin Endocrinol Metab. 2009 December; 94(12):4710-6. Epub 2009 Oct. 16.
Leblond, V. S., Bisson, M., Hontela, A., 2001. Inhibition of cortisol secretion in dispersed head kidney cells of rainbow trout (Oncorhynchus mykiss) by endosulfan, an organochlorine pesticide. General and Comparative Endocrinology. 121, 48-56.
Li, Y, Gilbert, T. R., Matsumoto, A. H. and Weibin, S. Effect of Aging on Fatty Streak Formation in a Diet-Induced Mouse Model of Atherosclerosis. J Vasc Res. 2008; 45(3): 205-210.
Maddox, S. A., Watts, C. S., Doyère, V. & Schafe, G. E. A naturally-occurring histone acetyltransferase inhibitor derived from Garcinia indica impairs newly acquired and reactivated fear memories. PloS One 8, e54463 (2013).
Merali, Z., Cayer, C., Kent, P. & Anisman, H. Nesfatin-1 increases anxiety- and fear-related behaviors in the rat. Psychopharmacology (Berl.) 201, 115-123 (2008).
Morgan, C. A., 3rd, Grillon, C., Southwick, S. M., Davis, M. & Charney, D. S. Fear-potentiated startle in posttraumatic stress disorder. Biol. Psychiatry 38, 378-385 (1995)
Mommsen, T. P., Moon, T. W., 1987. The metabolic potential of hepatocytes and kidney tissue in the little skate, Raja erinacea. Journal of Experimental Zoology. 244, 1-8.
Parker, K. J., Schatzberg, A. F., Lyons, D. M., 2003. Neuroendocrine aspects of hypercortisolism in major depression. Hormones and Behavior. 43, 60-66.
Passie, T., Emrich, H. M., Karst, M., Brandt, S. D. & Halpern, J. H. Mitigation of post-traumatic stress symptoms by Cannabis resin: a review of the clinical and neurobiological evidence. Drug Test. Anal. 4, 649-659 (2012).
Pellow. S., Chopin, P., File, S. E., Briley, M., 1985. Validation of open:closed arm entries in an elevated plus-maze as a measure of anxiety in the rat. Journal of Neuroscience Methods. 14, 149-167.
Rostagno, M. H., 2009. Can stress in farm animals increase food safety risk? Foodborne Pathogens and Disease. 6, 767-776.
Sarris, J., McIntyre, E. & Camfield, D. A. Plant-based medicines for anxiety disorders, part 2: a review of clinical studies with supporting preclinical evidence. CNS Drugs 27, 301-319 (2013).
Schreck, C. B., Contreras-Sanchez, W., Fitzpatrick, M. S., 2001. Effects of stress on fish reproduction, gamete quality, and progeny. Aquaculture. 197, 3-24.
Sharma, S. T., Nieman, L. K., 2011. Cushing's syndrome: All variants, detection, and treatment. Endocrinology and metabolism clinics of North America. 40, 379-391.
Smith, G. D., Ben-Shlomo, Y., Beswick, A., Yarnell, J., Lightman, S., Elwood, P., 2005. Cortisol, testosterone, and coronary heart disease: Prospective evidence from the caerphilly study. Circulation. 112, 332-340.
Steimer, T. Animal models of anxiety disorders in rats and mice: some conceptival issues. Dialogues Clin neurosci 2011, 13, 495-506.
Stewart, P. M., 2003. The adrenal cortex. in: Larsen, P. R., Kronenberg, H. M., Melmed, S., Polonsky, K. S. (Eds.), Williams textbook of endocrinology, 10th ed. Saunders, Philadelphia, pp. 491-551.
Thomson, F., Craighead, M., 2008. Innovative Approaches for the Treatment of Depression:Targeting the HPA Axis 33, 691.
Travison, T. G., O'Donnell, A. B., Araujo, A. B., Matsumoto, A. M., McKinlay, J. B., 2007. Cortisol levels and measures of body composition in middle-aged and older men. Clinical Endocrinology. 67, 71-77.
Wolkowitz, O. M., Reus, V. I., Chan, T., Manfredi, F., Raum, W., Johnson, R., Canick, J., 1999. Antiglucocorticoid treatment of depression: Double-blind ketoconazole. 45, 1070.
Zar, J. H., 1999. Biostatistical Analysis, Fourth Edition, Prentice Hall, Upper Saddle River, N.J.

1. A method for reducing glucocorticoids in an animal in need thereof, comprising administering to the animal a therapeutically effective amount of a compound of the formula (I): 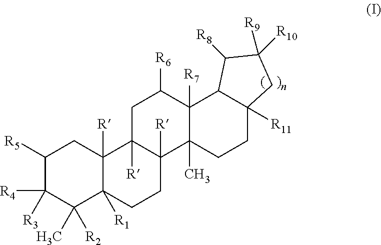
wherein
R′ are each independently or simultaneously H or CH₃;
R₁ is H or CH₃;
R₂ is H, CH₃ or CH₂OH;
R₃ is OH, —OCH₃, —OC(O)—(C₁₋₆)alkyl, —OC(O)—(C₆₋₁₀)aryl, —OC(O)—(C₆₋₁₀)heteroaryl, —NC(O)—(C₁₋₆)alkyl, —NC(O)-benzyl;
R₄ is H, —CH₃, (C₆-C₁₀)aryl or (C₆-C₁₀)heteroaryl; or
R₃ and R₄ are joined together to form C═O;
R₅ is H, OH, (C₁₋₆)alkyl or (C₂₋₆)alkenyl;
R₆ and R₇ are independently or simultaneously H or CH₃, or R₆ and R₇ are joined together to form a double bond (C═C);
R₈ is H, (C₁₋₆)alkyl, (C₂₋₆)alkenyl, (C₁₋₆)alkanol, (C₂₋₆)alkenol, —C(O)—(C₁₋₆)alkyl, —C(O)OH, —C(O)O—(C₁-C₆)alkyl, —C(O)—CH₂CH₂—(C₆-C₁₀)aryl, —C(O)—CH₂CH₂—(C₆-C₁₀)heteroaryl, —C(O)—(C₂-C₆)alkenyl, —C(O)—CH₂—CH═CH—(C₆-C₁₀)aryl, —C(O)—CH₂—CH═CH—(C₆-C₁₀)heteroaryl, 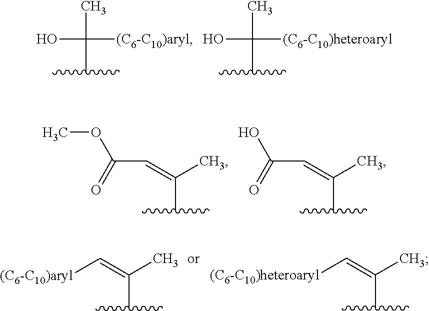
R₉ and R₁₀ are independently or simultaneously H or CH₃;
R₁₁ is CH₃, CH₂OH, CH₂OCOCH₃, —CH(O), —C(O)ORₐ, C(O)O—(C₁₋₆)alkyl-C(O)O—R_{b}, C(O)—NH—(C₁₋₆)alkyl-C(O)O—R_{b}; or —CH═CH—C(O)O—R_{b};
wherein Rₐ is H, (C₁₋₁₀)alkyl, (C₁₋₆)alkenyl, —CH₂—(C₆-C₁₀)-aryl, or —CH₂—(C₆-C₁₀)-heteroaryl;
R_{b} is H or (C₁₋₆)alkyl;
n is the integer 1 or 2; and
the aryl groups are optionally substituted with one to five substituents selected from halo, OH or (C₁₋₃)alkyl,
or a pharmaceutically acceptable salt or solvate thereof, and any stereoisomer thereof, with the proviso that the compound of the formula (I) is not

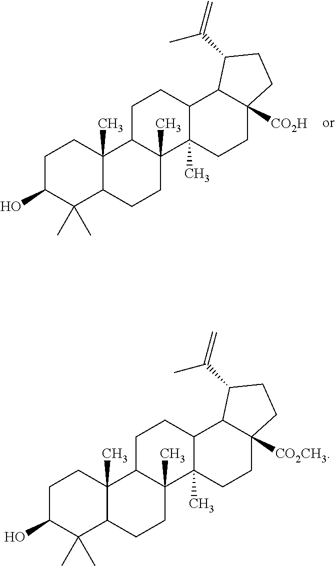

2. The method according to claim 1, wherein the compound of the formula (I) comprises a compound of the formula (Ia): 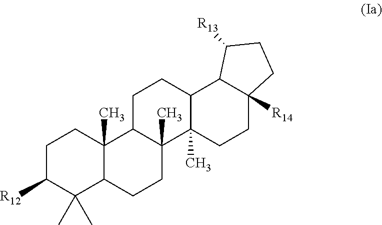
wherein
R₁₂ is OH, —O—CH₃ or —OC(O)CH₃;
R₁₃ is (C₁-C₆)alkyl, (C₂-C₆)alkenyl, (C₁-C₆)-alkanol, (C₂-C₆)-alkenol, —C(O)—(C₁₋₆)alkyl, —C(O)OH, —C(O)O—(C₁-C₆)alkyl, —C(O)—CH₂CH₂—(C₆-C₁₀)aryl, —C(O)—CH₂CH₂—(C₆-C₁₀)heteroaryl, —C(O)—(C₂-C₆)alkenyl, —C(O)—CH₂—CH═CH—(C₆-C₁₀)aryl, —C(O)—CH₂—CH═CH—(C₆-C₁₀)heteroaryl, 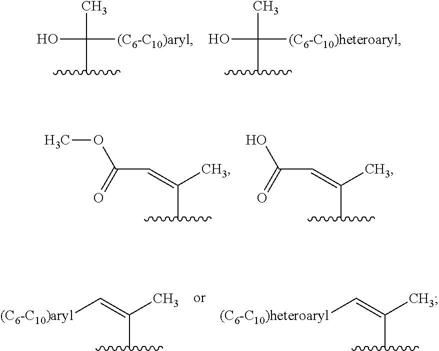
R₁₄ is CH₃, CH₂OH, CH₂OC(O)CH₃)—C(O)ORₐ or —C(O)O—(C₁₋₆)alkyl-C(O)O—R_{b}; and
wherein Rₐ is H, (C₁₋₁₀)alkyl, (C₁₋₆)alkenyl, —CH₂—(C₆-C₁₀)-aryl, or —CH₂—(C₆-C₁₀)-heteroaryl;
R_{b} is H or (C₁₋₆)alkyl.

3. The method according to claim 2, wherein
R₁₃ is —CO₂H, —CO₂CH₃, 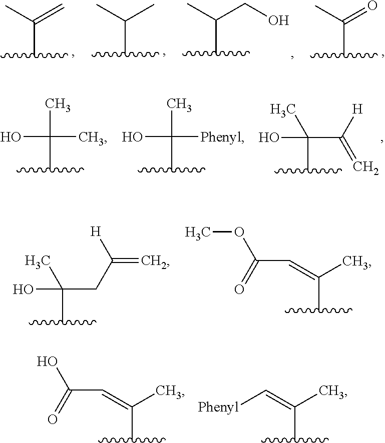
—C(O)—CH₂CH═CH₂, —C(O)—CH═CH—CH₃, —C(O)—CH₂CH₂-phenyl, or —C(O)—CH═CH— phenyl;
and
R₁₄ is CH₃, CH2OH, CH₂OC(O)CH—CO₂(benzyl), —CO₂—CH₂—CO₂CH₂CH₃, —CO₂H or —CO₂CH₃.

4. (canceled)

5. The method according to claim 4, wherein the compound of the formula (Ia) is

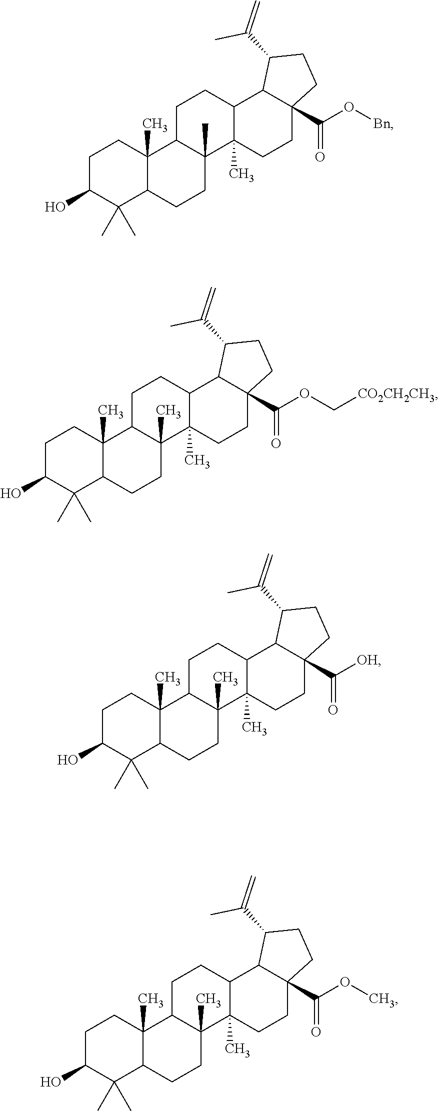

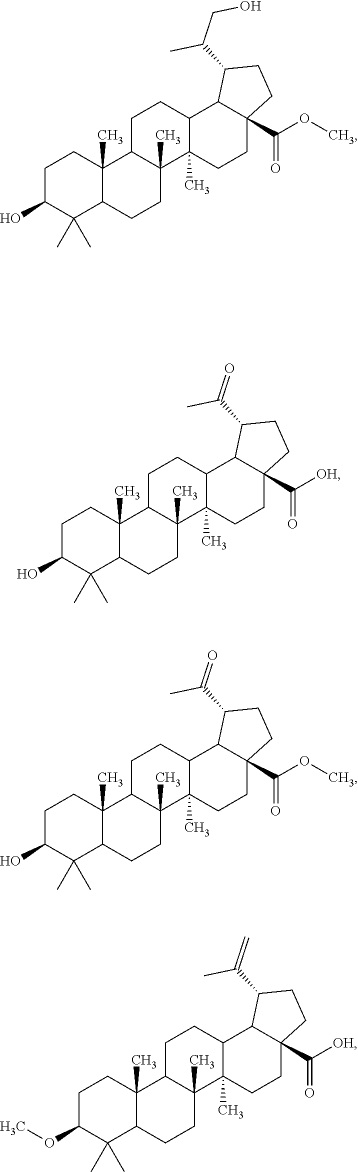

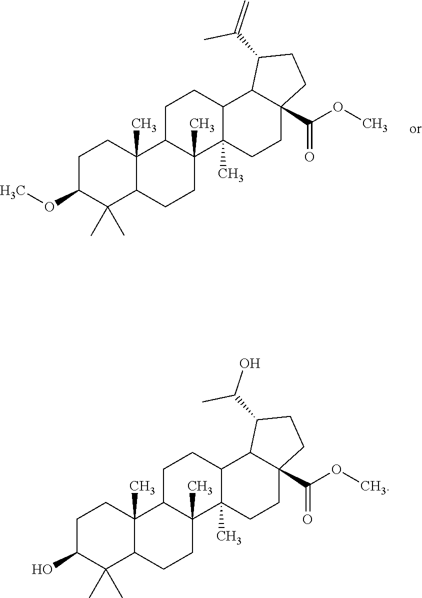

6. The method according to claim 1, wherein the compound of the formula (I) comprises a compound of the formula (Ib): 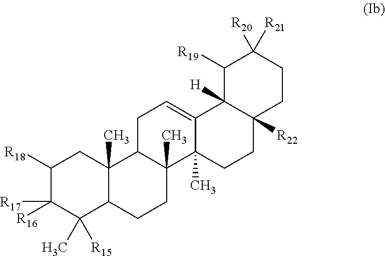
wherein
R₁₅ is CH₃, or CH₂OH or CH₂OC(O)CH₃;
R₁₆ is OH or —OC(O)CH₃;
R₁₇ is H; or
R₁₆ and R₁₇ are joined together to form C═O;
R₁₈ is H or OH;
R₁₉-R₂₁ are each simultaneously or independently H or CH₃; and
R₂₂ is CH₃, CH₂OH, CH₂OC(O)CH₃, —C(O)OH or —C(O)OCH₃.

7. The method according to claim 6, wherein the compound of the formula (Ib) comprises

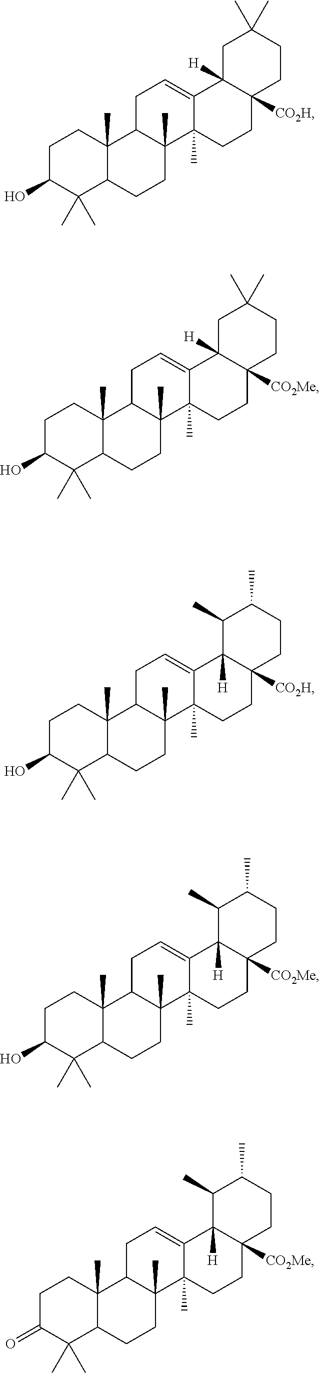

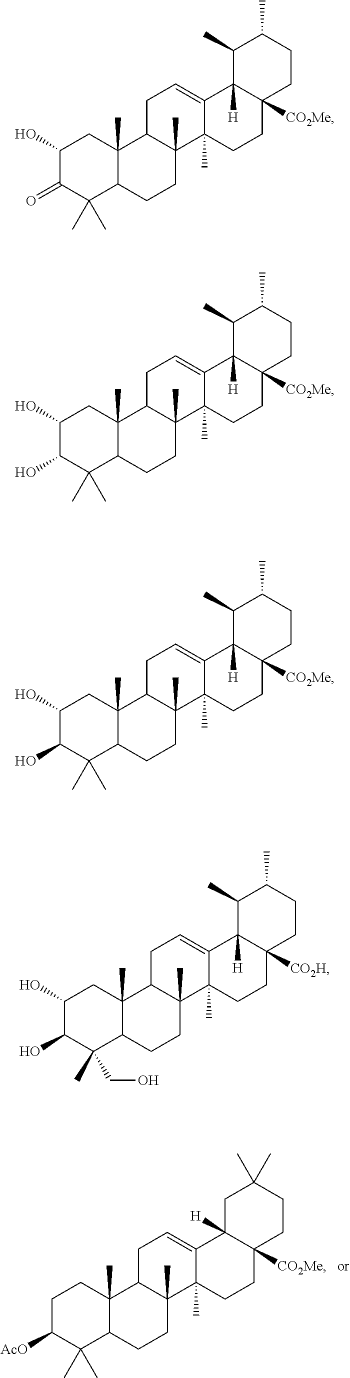

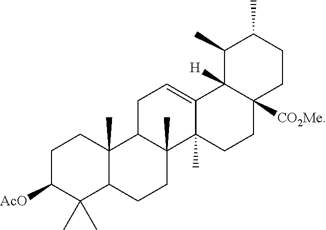

8. The method according to claim 1, wherein the compound of the formula (I) comprises a compound of the formula (Ic) 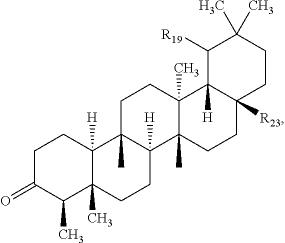
wherein R₂₃ is CH₃, CH₂OH, —C(O)OH or —C(O)OCH₃.

9. The method according to claim 7, wherein the compound of the formula (Ic) is

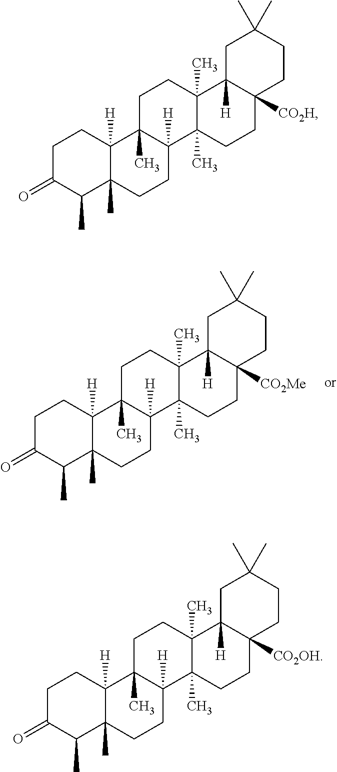

10. The method according to claim 1, wherein the compound of the formula (I) comprises a compound of the formula (Id) 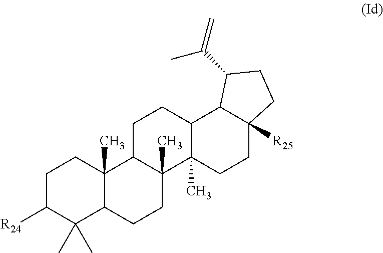
wherein
R₂₄ is —OC(O)—(C₁₋₆)alkyl, —OC(O)—(C₆₋₁₀)aryl, —NC(O)—(C₁₋₆)alkyl, —NC(O)-benzyl;
R₂₅ is —C(O)ORₐ;
wherein Rₐ is H or (C₁₋₁₀)alkyl,
wherein the alkyl and aryl groups are optionally substituted with one to five substituents selected from halo, OH or (C₁₋₃)alkyl.

11. (canceled)

12. The method according to claim 11, wherein the compound of the formula (Id) comprises

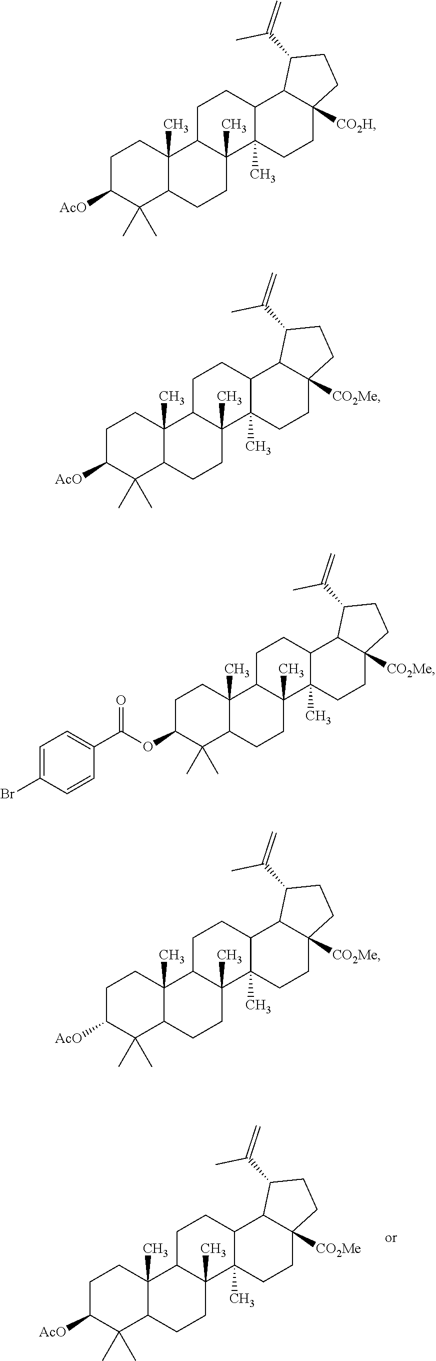

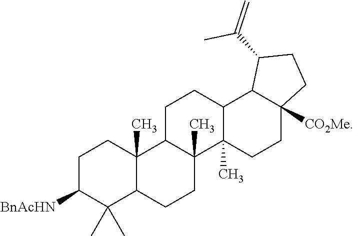

13. The method according to claim 1, wherein the compound of the formula (I) comprises a compound of the formula (Ie) 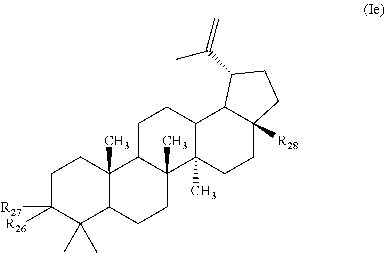
wherein
R₂₆ is OH;
R₂₇ is H; or
R₂₆ and R₂₇ are joined together to form C═O;
R₂₈ is CH₃, CH₂OH, CH₂OC(O)CH₃, CH(O), —C(O)ORₐ, —C(O)O—(C₁₋₆)alkyl-C(O)O—R_{b}, C(O)—NH—(C₁₋₆)alkyl-C(O)O—R_{b}, or —CH═CH—C(O)O—R_{b};
wherein Rₐ is (C₁₋₁₀)alkyl or (C₁₋₆)alkenyl,
R_{b} or H, (C₁₋₆)alkyl;
wherein the alkyl groups are optionally substituted with one to five substituents selected from halo, OH or (C₁₋₃)alkyl.

14. (canceled)

15. The method according to claim 12, wherein the compound of the formula (Ie) comprises

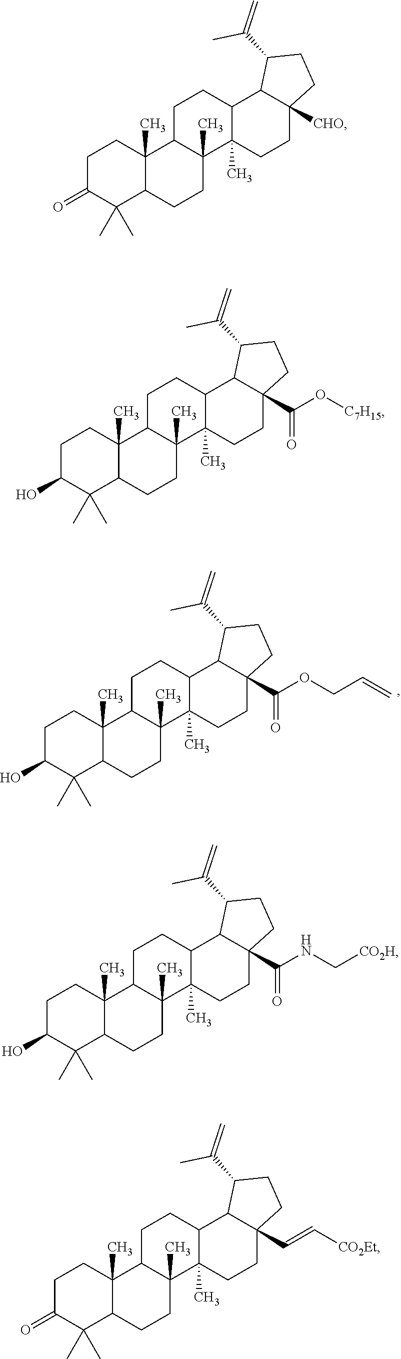

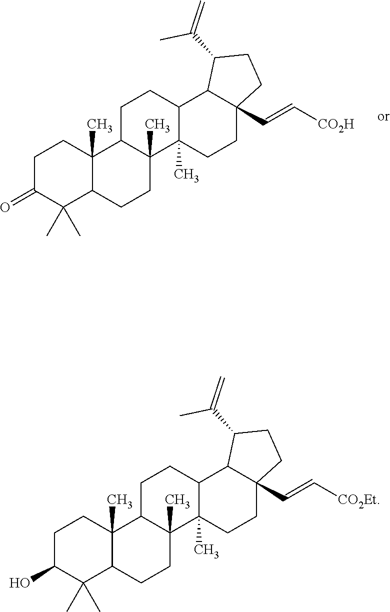

16. The method according to claim 1, wherein the compound of the formula (I) comprises a compound of the formula (If) 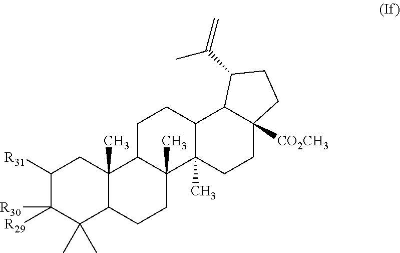
wherein
R₂₉ is OH;
R₃₀ is H; or
R₂₉ and R₃₀ are joined together to form C═O; and
R₃₁ is OH or (C₂₋₆)alkenyl.

17. The method according to claim 15, wherein the compound of the formula (If) is

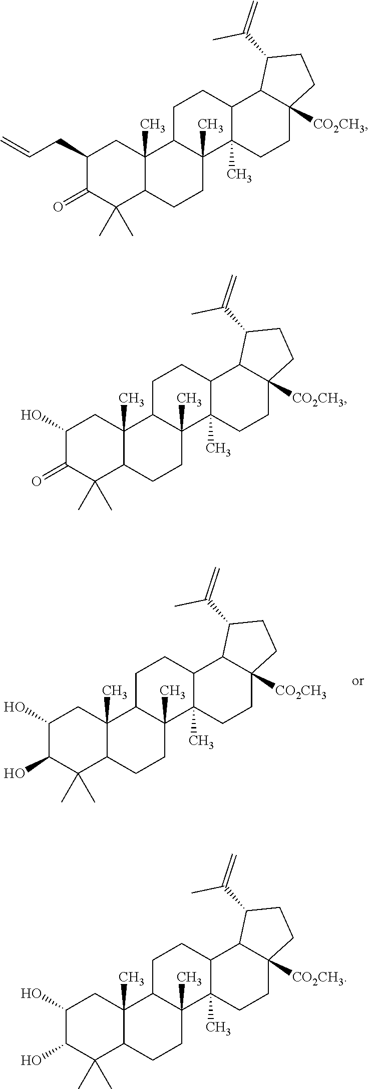

18. The method according to claim 1, wherein the compound of formula (I) comprises a compound of the formula (Ig) 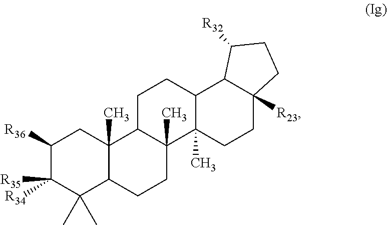
wherein
R₃₂ is —C(O)—(C₁₋₆)-alkanol, —(C₁₋₆)-alkanol, —(C₁₋₆)-dialkanol, or —C(O)—(C₁₋₆)-alkyl;
R₃₃ is —CO₂H, —C(O)N(R_{b})₂, or —C(O)NR_{b}—CH₂CO₂H;
R₃₄ is H;
R₃₅ is OH; or
R₃₄ and R₃₅ are joined together to form C═O;
R₃₆ is H or OH;
with the proviso that when R₃₂ is —(C₁₋₆)-alkanol or —C(O)—(C₁₋₆)-alkyl, R₃₆ is OH.

19.-20. (canceled)

21. The method according to claim 18, wherein the compound of the formula (Ig) is

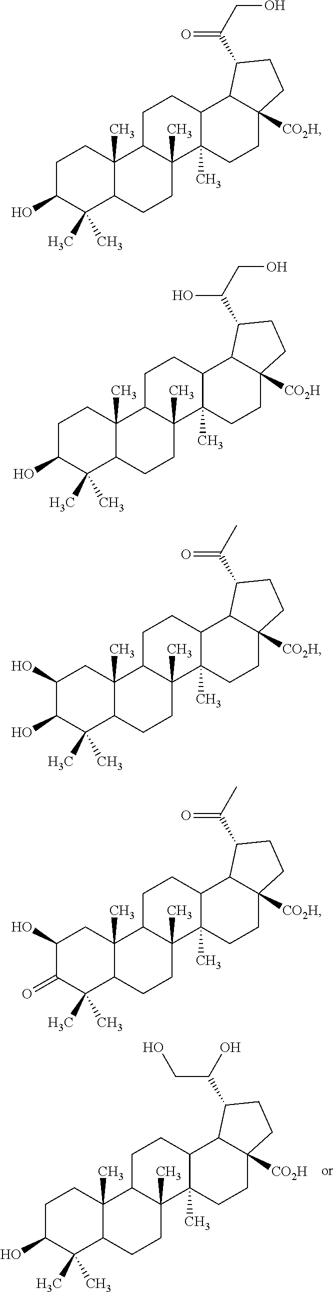

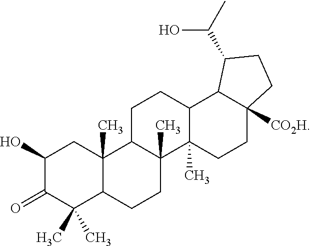

22. The method according to claim 1, for the treatment or prevention of a glucocorticoid-related disorder.

23. The method according to claim 1, for maintaining bone density, maintaining and improving the immune system, treating Cushing's syndrome, treating obesity, improving reproduction efficiency, treating metabolic disorder, treating hypertension, treating hyperglycemia, treating insulin resistance, treating type 2 diabetes, and/or aiding in cancer and immune therapies.

24. A pharmaceutical composition comprising a compound of the formula (IIa): 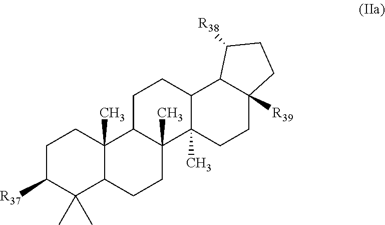
wherein
R₃₇ is OH, —O—CH₃ or —OC(O)CH₃;
R₃₈ is (C₁-C₆)alkyl, (C₂-C₆)alkenyl, (C₁-C₆)-alkanol, (C₂-C₆)-alkenol, —C(O)—(C₁₋₆)alkyl, —C(O)OH, —C(O)O—(C₁-C₆)alkyl, —C(O)—CH₂CH₂—(C₆-C₁₀)aryl, —C(O)—CH₂CH₂—(C₆-C₁₀)heteroaryl, —C(O)—(C₂-C₆)alkenyl, —C(O)—CH₂—CH═CH—(C₆-C₁₀)aryl, —C(O)—CH₂—CH═CH—(C₆-C₁₀)heteroaryl, 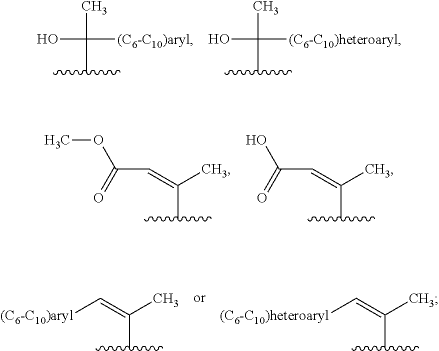
R₃₉ is CH₃, CH₂OH, CH₂OC(O)CH₃)—C(O)ORₐ or —C(O)O—(C₁₋₆)alkyl-C(O)O—R_{b}; and
wherein Rₐ is H, (C₁₋₁₀)alkenyl, —CH₂—(C₆-C₁₀)-aryl, or CH₂—(C₆-C₁₀)-heteroaryl;
R_{b} is H or (C₁₋₆)alkyl
or a pharmaceutically acceptable salt or solvate thereof, and any stereoisomer (entantiomer, diastereomer) thereof;
and
a compound of the formula (IIb): 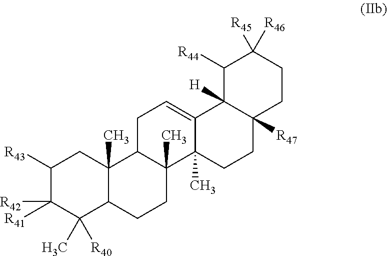
wherein
R₄₀ is CH₃, or CH₂OH or CH₂OC(O)CH₃;
R₄₁ is OH or —OC(O)CH₃;
R₄₂ is H; or
R₄₁ and R₄₂ are joined together to form C═O;
R₄₃ is H or OH;
R₄₄-R₄₆ are each simultaneously or independently H or CH₃; and
R₄₇ is CH₃, CH₂OH, CH₂OC(O)CH₃, —C(O)OH or —C(O)OCH₃,
or a pharmaceutically acceptable salt or solvate thereof, and any stereoisomer (entantiomer, diastereomer) thereof.

25. The pharmaceutical composition of claim 24, wherein the composition comprises

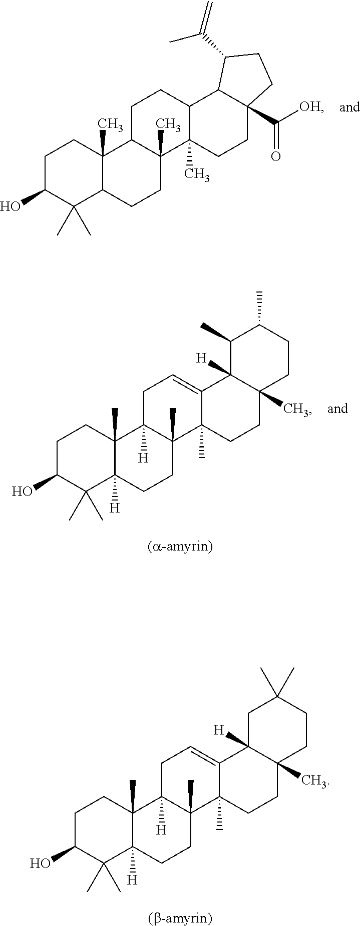

26. A method for reducing glucocorticoids and/or treating anxiety in an animal in need thereof, comprising administering to the animal a therapeutically effective amount of a pharmaceutical composition as claimed in claim 24.

27. The method of claim 26 for maintaining bone density, maintaining and improving the immune system, treating Cushing's syndrome, reducing aggression and hyperactivity, treating obesity, improving reproduction efficiency, treating metabolic disorder, treating hypertension, treating hyperglycemia, treating insulin resistance, treating type 2 diabetes, for treating post-traumatic stress disorder, and/or aiding in cancer and immune therapies.
